(19) United States
(12) Patent Application Publication
(10) Pub. No.: US 2025/0214047 A1
(43) Pub. Date: Jul. 3, 2025

(54) A TECHNOLOGY FOR COMBINATORIAL LIQUID HANDLING

(71) Applicant: BIOSISTEMIKA D.O.O., Ljubljana (SI)

(72) Inventors: Urška CEPIN, Ig (SI); Tomaž KARCNIK, Ljubljana (SI); Tadej KOKALJ, Trzin (SI); Rok LUZAR, Mengeš (SI); Zala PREVORŠEK, Škofja Loka (SI); Tjaša STARE, Ljubljana (SI); Borut ŠOLAR, Žabnica (SI); Luka ZUPANCIC, Ljubljana (SI); Valentina JANCIC, Prestranek (SI)

(73) Assignee: BIOSISTEMIKA D.O.O., Ljubljana (SI)

(21) Appl. No.: 18/852,654

(22) PCT Filed: Mar. 30, 2023
(86) PCT No.: PCT/EP2023/058421

§ 371 Date: Sep. 30, 2024

(30) Foreign Application Priority Data
Apr. 1, 2022 (EP) 22166355.2
Apr. 1, 2022 (EP) 22166357.8
May 6, 2022 (LU) LU502028
May 6, 2022 (LU) LU502029
May 6, 2022 (LU) LU502031
May 6, 2022 (LU) LU502033

(51) Int. Cl. B01F 33/302; A61J 3/00; B01F 23/41; B01F 23/411; B01F 33/301; B01F 33/3031; B01F 101/00; B01F 101/22; B29C 64/112; B29C 64/209; B29K 105/00; B29L 31/00; B33Y 10/00; B33Y 30/00; B33Y 80/00; C12N 5/00

(52) U.S. Cl. CPC B01F 33/3021; A61J 3/007; B01F 23/4105; B01F 23/411; B01F 23/4143; B01F 33/301; B01F 33/3031; B29C 64/112; B29C 64/209; B33Y 10/00; B33Y 30/00; B33Y 80/00; C12N 5/0062; B01F 2101/22; B01F 2101/2202; B01F 2215/045; B29K 2105/0035; B29L 2031/753

(57) ABSTRACT

Various devices and methods covering a technology for combinatorial liquid handling. At least some example embodiments of the devices may include a plurality of liquid dispensers configured to dispense drops, wherein at least a carrier drop is dispensed and sequentially collides and merges with drops dispensed from a sub-plurality of liquid dispensers, so that liquids from the sub-plurality of liquid dispensers are located in the carrier drop during flight, and a support surface being positioned so that the carrier drop lands on the support surface.

The project DATANA leading to this application has received funding from the European Union's Horizon 2020 research and innovation programme under grant agreement No 970550.

### TECHNICAL FIELDS

In the present disclosure, various devices and methods covering a technology are disclosed. The technology according to the present disclosure relates to combinatorial liquid handling.

Some devices and methods of the present disclosure relate to combinatorial liquid handling, more specifically, producing liquid mixtures in a combinatorial manner. Some devices and methods of the present disclosure relate to depositing biological materials and/or biomaterials, more specifically, preparing samples for assays. Some devices and methods of the present disclosure relate to bioprinting, more specifically, bioprinting of multi-component 3D objects such as organs on a chip, tissues, organs, etc. Some devices and methods of the present disclosure relate to additive manufacturing, more specifically, additive manufacturing of multi-material 3D objects from metal and/or ceramic powders suspended in liquids. Some devices and methods of the present disclosure relate to manufacturing pharmaceutical products, more specifically, manufacturing tablets for oral or buccal delivery. Some devices and methods of the present disclosure relate to manufacturing pharmaceutical products, more specifically, manufacturing of spherically shaped micro and/or nano sized therapeutical particles for parerental, respiratory or nasal delivery. Some devices and methods disclosed herein relate to coating devices, more specifically, devices for applying active pharmaceutical ingredients on medical devices such as implants, micro-needle patches, etc. Some devices and methods of the present disclosure relate to manufacturing pharmaceutical products, more specifically, applying active pharmaceutical ingredients on placebo pharmaceuticals. Some devices and methods of the present disclosure relate to manufacturing pharmaceutical products, more specifically, assembly of nucleic acids for pharmaceutical, phytopharmaceutical or veterinary use.

### BACKGROUND

Liquid dispensing, handling and mixing devices come in many forms, shapes and sizes, and are used in various fields of industry. Liquid dispensing, handling and mixing devices play an important role in pharmaceutical industry, biotechnological industry, chemical industry, in additive manufacturing, as well as other industries, and are used for both industrial and research purposes. Many of these devices are aimed at replacing manual labour of applying a plurality of liquids in one location, which may be specifically troublesome whilst applying and mixing low volumes of liquids. Also, the process becomes even more time-consuming and technically challenging for producing liquid mixtures or solid compounds from a larger number of distinct liquids. More and more complex liquid dispensing devices and methods have therefore been developed recently for various different purposes of use in various different fields of industry.

In recent years, the trend towards production of customized compounds is rising in many different fields of industry. For example, a trend towards the production of customised therapeutical products, such as tablets, vaccines, nucleic acids, or other therapeutical products, is gaining a specific importance. With a rise of innovative approaches in medicine, such as customizing drugs for each patient, devices capable of mixing larger numbers of various different liquids intended for mixing of active pharmaceutical ingredients and excipients to produce custom therapeutical products may be beneficial. With a customised approach aimed at each patient, custom active pharmaceutical ingredients may be produced to fit each patient individually. Liquid dispensers may also be used to coat medical devices, as well as for application of active pharmaceutical ingredients on placebos. As new compounds and also materials are being developed every day, and many of said new compounds require small amounts of liquids to be mixed together, the need for more precise liquid dispensers and mixers is ever increasing. Furthermore, an increasingly larger number of substances may be mixed together to create customized mixtures, and therefore, the need for liquid handling devices, which are capable of mixing large number of compounds is also ever increasing. In biotechnology, liquid handling devices play an important role in preparing samples of biological materials and biomaterials for assays. Increasingly larger numbers of substances are of interest in the recent years in the fields of biotechnology, and devices to handle larger number of liquids that may contain substances of interest may be beneficial in various fields. Liquid dispensers are also used in devices and methods for additive manufacturing and bioprinting, a trend which is also ever increasing. As today even human tissues and organs may already be bioprinted, liquid handling devices are also used in bioprinting. Liquid handling devices are also used in additive manufacturing of 3D objects from metal or ceramic powders suspended in liquids (such as polymers or binder materials).

In recent years, therefore, we have seen the development of many liquid micro-droplet dispensers, automated pipetting tools, pipetting robots, and similar tools to aid the mixing of large numbers of various different liquids. Furthermore, combinatorial mixing devices and methods emerged. Combinatorial mixing of compounds is used in many fields, for example in combinatorial chemistry, combinatorial biology, and other fields. Combinatorial mixing has been a large part of a drug discovery process in the pharmaceutical industry, combinatorial chemistry has also been used in new material science.

We shall hereinafter discuss some of the publicly described technical solutions used for handling liquids. In general terms, we may distinguish between contact mixing devices and devices for non-contact mixing of liquids. Hereinafter some of the selected non-contact liquid mixing devices will be described in detail.

Document U.S. Pat. No. 6,200,013B1 describes a device for uniform mixing of materials, which comprises two or more droplet-discharging means each having dedicated material sources, and at least one recovery container. Droplets discharged from a first droplet-discharging means and droplets discharged from a second droplet-discharging means can be collided to form an admixture or a reaction product which is collected in the recovery container, whereas fine droplets from any of said droplet-discharging means which do not collide with any fine droplets from any other said droplet-discharging means are not collected in said recovery container.

The document WO2016096054A1 describes a device for generating and mixing a first stream of droplets with at least one second stream of droplets to obtain a third stream of fused droplets comprising: a first dispenser for generating the first stream of droplets; at least one a second dispenser for generating at least one second stream of droplets, holding means for holding the first and at least one second dispenser, wherein the first and/or the at least one second dispenser preferably is/are (a) piezoelectric or thermal inkjet dispenser(s). The device further comprises synchronizing means for synchronizing the operation of the first and the at least one second dispenser, to effect collision and fusion of at least one droplet of the first stream with at least one droplet of the second stream to obtain the third stream.

At least some example embodiments of the devices and the methods according to the present disclosure relate to solving a problem of rapid contactless high throughput formation of a stream of unique drops, wherein each drop may include a unique and highly complex mixture of liquid materials before the drop lands, and wherein the unique and highly complex mixture is created in each drop in the stream by in-flight collision of a carrier drop with a plurality of drops that aggregately comprise one subset of liquids from a set of liquids used with the device.

In the devices and methods according to the present disclosure, one liquid dispenser dispenses a carrier drop in the direction along an array of at least at least two liquid dispensers. Drops are dispensed from the liquid dispensers in the array towards a trajectory of the carrier drop in a synchronised manner, so that the carrier drop collides and merges with drops dispensed from the array, and in this way, liquids included in the drops dispensed from the array may be positioned and located in the carrier drop, before the carrier drops lands on a support surface, and due to controlled collisions between the carrier drop and a plurality of drops dispensed from the array. In a preferred embodiment, two hundred liquid dispensers are included in an linear array, and the carrier drop passes by all two hundred nozzles in the liquid dispensers positioned in the array in close proximity. A rapid stream of carrier drops may be produced wherein each carrier drop may be collided and merged with a unique subset of liquids from the set of liquids held in a plurality of liquid dispensers in the device. In this way, each carrier drop in the stream may collect a unique mixture of liquids dispensed from the array, by using collisions between the carrier drops and the drops dispensed from the array. In this way, any selected combination of liquids from the set may be mixed together in one step, in one carrier drop, during a flight of the carrier drop.

Advantageous effects of invention may include creating highly complex liquid mixtures in-flight in the carrier drops, and depositing the highly complex liquid mixtures located in carrier drops on the support surface. The carrier drops may be deposited one next to another, so that two carrier drops that hold two unique liquid mixtures created in-flight may be deposited one next to another in close proximity. In this way, two or more highly complex mixtures of liquids, which may carry component materials, may be deposited in close proximity. Such a technical solution enables the production of highly complex liquid mixtures that may be deposited next to each other at two successive coordinates on the support surface. In some example embodiments the highly complex liquid mixtures may be deposited at some select XY coordinates on the support surface, or for example, in a series of successive XYZ coordinates on the support surface. This enables highly complex combinatorial depositing of materials dispensed in liquids from the set, and in some examples, manufacturing or bioprinting of complex multi-material 3D objects.

### SUMMARY

At least some example embodiments of devices and methods according to the present disclosure relate to a device and a method for mixing liquids. Hereinafter some example embodiments of devices and methods that relate to the device and the method for mixing liquids are summarised. The technical problem relating to combinatorial mixing of liquids is solved by the device for mixing liquids according to claim 1 and by the method for mixing liquids according to claim 14. In at least some example embodiments, the device for mixing liquid (hereinafter: the device) may comprise a plurality of liquid dispensers configured to dispense drops, wherein at least a carrier drop is dispensed and sequentially collides and merges with drops dispensed from a sub-plurality of liquid dispensers, so that liquids from the sub-plurality of liquid dispensers are located in the carrier drop, and a support surface being positioned so that the carrier drop lands on the support surface. Each of the liquid dispensers in the plurality of liquid dispensers may comprise at least a container for holding liquids to dispense, a drop generating element positioned and configured to cause at least one drop to dispense, a nozzle through which the at least one drop is ejected from, a capillary which connects the container with the nozzle so that the liquid runs from the container through the capillary and out through the nozzle for the at least one drop to be dispensed, and a synchronisation means, which enable each of the liquid dispensers to operate in a synchronised manner with other liquid dispensers in the plurality of liquid dispensers. In some example embodiments of the device, a single container may be configured to supply liquids to two or more nozzles in the plurality of liquid dispensers. In some example embodiments of the device, two or more containers may be configured to supply liquids to a single nozzle in the plurality of liquid dispensers. Each of the liquid dispensers in the device may be being configured to dispense up to a hundred thousand (100000) drops per second. Each of the liquid dispensers in the device may be configured to dispense drops with a volume in a range between 1 picoliter to 100 nanoliters. The device may comprise fifty or more, or five hundred or more, or ten thousands or more liquid dispensers.

At least one liquid dispenser in the device is configured to dispense the carrier drop. The carrier drop may have a trajectory that passes by two or more liquid dispensers configured to dispense drops towards the trajectory of the carrier drop in a synchronised manner, so that the carrier drop sequentially collides with at least all of the drops dispensed by at least some of the two or more liquid dispensers.

In at least some example embodiments of the device, the device may comprise at least one liquid dispenser being configured to dispense a stream of carrier drops, wherein the stream comprises a first carrier drop, a second carrier drop, and at least a third carrier drop, and the plurality of liquid dispensers may be configured so that the first carrier drop collides with drops from a first sub-plurality of liquid dispensers, the second carrier drop collides with drops from a second sub-plurality of liquid dispensers, the third carrier drop collides with drops from a third sub-plurality of liquid dispensers, and so on, until all of the carrier drops in the stream collide with at least some drops dispensed from at least some liquid dispensers from the plurality of liquid dispensers, and all of the carrier drops in the stream land on the support surface.

In at least some example embodiments of the device, the device may comprise two or more liquid dispensers arranged in an array, such as a linear array, a circular array, a mesh array, or any other suitable type of array comprising two or more liquid dispensers arranged in an ordered manner. In at least some example embodiments of the device, the device may comprise at least some of the liquid dispensers arranged in at least a first array and a second array, wherein the first array and the second array are configured to dispense drops towards a single stream of the carrier drops. In at least some example embodiments of the device, the device may comprise the first array being configured to dispense drops towards a first stream of carrier drops, and the second array being configured to dispense drops towards a second stream of carrier drops.

In at least some example embodiments of the device, the support surface may be made of a bendable material or a non-bendable material. In at least some example embodiments of the device, the support surface may be detachable or non-detachable relative to the device. In at least some example embodiments of the device, the support surface is a container filled with a liquid in which the carrier drops land. In at least some example embodiments of the device, the support surface is a flat surface. In at least some example embodiments of the device, the device may comprise the plurality of liquid dispensers being configured to dispense drops of a first liquid, and the support surface is filled or covered with a second liquid, and the first liquid and the second liquid are immiscible.

In at least some example embodiments of the device, the carrier drop includes anti-solvent liquids, such as: water, tert-butanol, ethanol, and the liquids from the sub-plurality of liquid dispensers in dispensed drops (and which may include materials or components) that collide with the carrier drop do not solve in the carrier drop.

In at least some example embodiments of the device, the device may comprise a mixing chamber, which is a partially or a fully closed compartment of the device wherein the plurality of liquid dispensers and the support surface are positioned, and the mixing chamber is the fully closed compartment during the time in which the drops are in-flight.

In at least some example embodiments of the device, the device may comprise electric plates arranged perpendicularly to a path of at least one drop dispensed from at least one liquid dispenser, wherein the electric plates have holes through which the at least one drop passes, the at least one liquid dispenser is configured to provide an electric charge to the at least one drop, and the electric plates are configured so that the electric plates exert electric fields on the at least one drop in a synchronised manner, so that the at least one drop is manipulated with the electric fields exerted by the electric plates during the time in which the at least one drop passes through the electric fields exerted by the electric plates.

In at least some example embodiments of the device, the device may comprise a processing chamber having at least one processing element configured to remove the anti-solvent liquids from each carrier drop that includes the anti-solvent liquids and is located in the processing chamber.

In at least some example embodiments of the device, the device may comprise a mechanisation configured to move the support surface in at least one direction relative to the plurality of liquid dispensers, and/or move the plurality of liquid dispensers in at least one direction relative to the support surface, and/or move the support surface from the mixing chamber to the processing chamber.

In at least some example embodiments of the method for mixing liquids, the method for mixing liquids comprises dispensing, by using a plurality of liquid dispensers, at least one carrier drop and at least drops that comprise liquids from a select sub-plurality of liquid dispensers, wherein the carrier drop sequentially collides and merges with the drops that comprise the liquids from the select sub-plurality of liquid dispensers, so that the liquids from the select sub-plurality of liquid dispensers are located in the carrier drop during flight, and collecting the at least one carrier drop by using a support surface.

In at least some example embodiments of the method for mixing liquids, the method for mixing liquids includes using any of the example embodiments of the devices as described herein.

At least some example embodiments of devices and methods according to the present disclosure relate to a device and a method for preparing samples for assays. Hereinafter some example embodiments of the devices and the methods that relate to the device and the method for preparing samples for assays are summarised. The technical problem relating preparing samples for assays is solved by the device for preparing samples for assays (hereinafter: the device) according to claim 16 and by the method for preparing samples for assays according to claim 33.

In at least some example embodiments of the device, the device may comprise a plurality of liquid dispensers being configured to dispense drops, so that at least a carrier drop sequentially collides with N drops that comprise a first substance and M drops that comprise at least a second substance, so that the first substance and the second substance are located at a ratio of interest (N:M) and at an amount of interest (N+M) in the carrier drop during flight, and a support surface being configured to collect the carrier drop on a select XY coordinate on the support surface, and in this way, the samples are prepared for the assay on the support surface. Each of the liquid dispensers in the plurality of liquid dispensers may comprise at least a container for holding liquids to dispense, a drop generating element positioned and configured to cause at least one drop to dispense, a nozzle through which the at least one drop is ejected from, a capillary which connects the container with the nozzle so that the liquid runs from the container through the capillary and out through the nozzle for the at least one drop to be dispensed, and a synchronisation means, which enable each of the liquid dispensers to operate in a synchronised manner with other liquid dispensers in the plurality of liquid dispensers. In some example embodiments of the device, a single container may be configured to supply liquids to two or more nozzles in the plurality of liquid dispensers. In some example embodiments of the device, two or more containers may be configured to supply liquids to a single nozzle in the plurality of liquid dispensers. Each of the liquid dispensers in the device may be being configured to dispense up to a hundred thousand (100000) drops per second. Each of the liquid dispensers in the device may be configured to dispense drops with a volume in a range between 1 picoliter to 100 nanoliters. The device may comprise fifty or more, or five hundred or more, or ten thousands or more liquid dispensers.

A least one liquid dispenser in the device is configured to dispense the carrier drop. The carrier drop may have a trajectory that passes by two or more liquid dispensers configured to dispense drops towards the trajectory of the carrier drop in a synchronised manner, so that the carrier drop sequentially collides with at least the N drops and the M drops, which are dispensed by at least some of the two or more liquid dispensers.

In at least some example embodiments of the device, the device may comprise at least one liquid dispenser being configured to dispense a stream of carrier drops, wherein the stream comprises a first carrier drop, a second carrier drop, and at least a third carrier drop, and the plurality of liquid dispensers are configured so that the first carrier drop collides a first number of the N drops and the M drops, the second carrier drop collides with a second number of the N drops and the M drops, the third carrier drop collides with a third number of the N drops and the M drops, and so on, until all of the carrier drops in the stream collide with at least some number of the N drops and the M drops, and all of the carrier drops in the stream land on the support surface.

In at least some example embodiments of the device, the device may comprise the support surface being configured to collect each carrier drop in the stream at a unique select XY coordinate on the support surface, or to collect two or more carrier drops at a same select XY coordinate on the support surface, depending on the amount of interest (N+M) of the first substance and the second substance to be prepared.

In at least some example embodiments of the device, the device may comprise two or more liquid dispensers arranged in an array, such as a linear array or a circular array. In at least some example embodiments of the device, the device may comprise at least some of the liquid dispensers arranged in at least a first array and a second array, wherein the first array and the second array are configured to dispense drops towards a single stream of the carrier drops. In at least some example embodiments of the device, the device may comprise the first array is configured to dispense drops towards a first stream of carrier drops, and the second array is configured to dispense drops towards a second stream of carrier drops.

In at least some example embodiments of the device, the device may comprise at least one liquid dispenser being configured to dispense drops that contain no substances of interest, and the plurality of liquid dispensers is configured to collide the carrier drop with at least some of the drops that contain no substances of interest, so that a lower concentration of the first substance and at least the second substance is achieved in the carrier drop, or no substances are contained in the carrier drop when the carrier drop lands, so that the carrier drop, which contains no substances, is used for control purposes in the sample, which is prepared.

In at least some example embodiments of the device, the device may comprise at least some of liquid dispensers being configured to dispense drops that collide with the carrier drops and aggregately comprise more than two substances of interest, so that the more than two substances substances are prepared at the ratio of interest, the amount of interest, and concentration of interest, at all available select XY coordinates on the support surface.

In at least some example embodiments of the device, the device may be configured to prepare the sample on all available select XY coordinates on the support surface, so that the all available select XY coordinates on the support surface are filled with at least two substances at the ratio of interest, the amount of interest, and a concentration of interest, and/or no substances of interest.

In at least some example embodiments of the device, the first substance, the second substance, and/or any other substance to be prepared in the sample, are substances, such as: soluble proteins, chemical additives, liquid substances used for pH control of the sample, isolated mRNA of interest, isolated gDNA of interest, isolated DNA of interest, isolated RNA of interest, regulatory plasmids, at least one DNA fragment (such as: siRNA), primers, probes, RT-qPCR Master Mix, qPCR Master mix, Master Mix, a desired concentration of APIs, solvent, anti-solvent dispersion (such as: methanol, ethanol, water, dimethyl sulfoxide (DMSO), ethyl acetate, toluene), polymers (such as: hydroxypropyl methyl cellulose (HPMC), poly vinyl alcohol (PVA), Eudragit® derivates, polyvinylpyrroldone (PVP), polyethylene glycol (PEG)), pharma polymers, surfactants (such as: polysorbate 20 Tween—Tween 20®, polysorbate 80—Tween 80®), humectants (such as: PEG, glycerol), at least one viscosity modifier (such as: PEG, glycerol), cells (such as: cells with excreted proteases, mammalian cells, E. coli cells, yeast cells, microbial cells), protein biomarkers (such as: protein metalloproteinase (MMP), various FRET-based protease substrates with or without specific inhibitors), cell encapsulation polymer used for cell encapsulation, pathogens, growth factors, metabolites, chemical of interest, a liquid used for achieving a varying concentration of any of the components listed herein, primer beads, PCR inhibitors, plasmids (such as: linearised vector encoding for bait (such as pGBKT7) protein and pray (such pGADT7) protein), DNA fragment encoding for two potential interactors (proteins) of interest, linearised vectors, fungal solution, antifungal components, at least one type of enzymes (such as HRP, ligase, polymerase), antibodies, chemical sensors, biosensors, or any other substance of interest.

In at least some example embodiments of the device, support surface may be made of a bendable material or a non-bendable material, and/or may be detachable or non-detachable relative to the device, and/or may be a container filled with a liquid in which the carrier drops land, and/or is a surface.

In at least some example embodiments of the device, the device may comprise a mechanisation configured to move the support surface in at least one direction relative to the plurality of liquid dispensers, and/or to move the plurality of liquid dispensers in at least one direction relative to the support surface, so that the carrier drops land at the select XY coordinates on the support surface.

In at least some example embodiments of the device, the support surface holds a standard microtiter plate or any other plastic consumable, which are used with the device, so that the sample is prepared on the select XY coordinates that correspond to select XY locations of wells in the microtiter plate or in/on the any other plastic consumable, which is positioned on the support surface.

In at least some example embodiments of the device, the device is used to prepare the sample for the assay (analysis), such as: a high-throughput protein crystallography screening, qPCR for gene-expression studies or qPCR for SNP genotyping, ddPCR assays, a polymorph screening of engineered particles and/or compounds, any other molecular-based assays, cell encapsulation into multi-component materials studies, a single-cell line viability screening, a multiplex screening of biomarkers in cells, compound libraries screening, high-throughput forensic genotyping—Short Tandem Repeat (STR), gene expression regulation assay, ADMET (Absorption, Distribution, Metabolism, Elimination, Toxicity) assay, protein-protein interaction assays using yeast two-hybrid system, protein-DNA interaction assays using yeast one-hybrid system, microorganism activity/sensitivity assessment screening, antifungal screening on a chip, other assays wherein microorganisms are screened, biosensor validation and screening assays, rapid automated immunoassay or any other assay of interest.

In at least some example embodiments of the device, the device may comprise a processing chamber wherein at least one environment regulator is installed and configured to regulate at least temperature, humidity, and CO₂ concentration in the processing chamber, so that the sample, which was prepared on the support surface and is located in the processing chamber, is incubated.

In at least some example embodiments of the device, the device may comprise electric plates arranged perpendicularly to a path of at least one drop dispensed from at least one liquid dispenser, wherein the electric plates have holes through which the at least one drop passes, the at least one liquid dispenser is configured to provide an electric charge to the at least one drop, and the electric plates are configured so that the electric plates exert electric fields on the at least one drop in a synchronised manner, so that the at least one drop is manipulated with the electric fields exerted by the electric plates during the time in which the at least one drop passes through the electric fields exerted by the electric plates.

In at least some example embodiments of the method for preparing samples for assays (hereinafter: the method), the method includes dispensing and colliding drops by using a plurality of liquid dispensers, so that at least one carrier drop sequentially collides with N drops that comprise a first substance and M drops that comprise at least a second substance, so that the first substance and the second substance are located at a ratio of interest (N:M) and at an amount of interest (N+M) in the carrier drop during flight, and configuring the support surface to so that the carrier drop lands at a select XY coordinate on the support surface and samples are prepared for the assay on the support surface this way.

In at least some example embodiments of the method, the method includes using any one of the devices according to the present disclosure.

At least some example embodiments of devices and methods according to the present disclosure relate to a device and a method for bioprinting multi-component 3D objects. Hereinafter some example embodiments of the devices and the methods that relate to the device and the method for bioprinting multi-component 3D objects are summarised. The technical problem relating to bioprinting of multi-component 3D objects is solved by the device for bioprinting multi-component 3D objects (hereinafter: the device) according to claim 35 and by the method for bioprinting multi-component 3D objects according to claim 47.

In at least some example embodiments of the device, the device may comprise a plurality of liquid dispensers being configured to dispense and collide drops, wherein a stream of n carrier drops is dispensed and each of the n carrier drops in the stream sequentially collides with at least drops that aggregately comprise one subset of components from a set, and a support surface being configured so that the n carrier drops in the stream land in a series of n successive XYZ coordinates that represent the multi-component 3D object, so that the multi-component 3D object is bioprinted on the support surface. Each of the liquid dispensers in the plurality of liquid dispensers may comprise at least a container for holding liquids to dispense, a drop generating element positioned and configured to cause at least one drop to dispense, a nozzle through which the at least one drop is ejected from, a capillary which connects the container with the nozzle so that the liquid runs from the container through the capillary and out through the nozzle for the at least one drop to be dispensed, and a synchronisation means, which enable each of the liquid dispensers to operate in a synchronised manner with other liquid dispensers in the plurality of liquid dispensers. In some example embodiments of the device, a single container may be configured to supply liquids to two or more nozzles in the plurality of liquid dispensers. In some example embodiments of the device, two or more containers may be configured to supply liquids to a single nozzle in the plurality of liquid dispensers. Each of the liquid dispensers in the device may be being configured to dispense up to a hundred thousand (100000) drops per second. Each of the liquid dispensers in the device may be configured to dispense drops with a volume in a range between 1 picoliter to 1000 nanoliters. The device may comprise fifty or more, or five hundred or more, or ten thousands or more liquid dispensers.

In at least some example embodiments of the device, the device may comprise at least one liquid dispenser is configured to dispense the stream of the n carrier drops, wherein the stream comprises a first carrier drop, a second carrier drop, and at least a third carrier drop, and the plurality of liquid dispensers are configured so that the first carrier drop collides with at least drops that aggregately comprise a first subset of components, the second carrier drop collides with at least drops that aggregately comprise a second subset of components, the third carrier drop collides with at least drops that aggregately comprise a third subset components from, and so on, until all of the n carrier drops in the stream collide with at least some drops that aggregately comprise at least some appropriate components from the set, and all of the n carrier drops in the stream land on the support surface.

In at least some example embodiments of the device, the n carrier drops include a cell-carrying bio-ink, such as a biopolymer gel, at the time of dispensing, or the n carrier drops include at least one type of cells suspended in the cell-carrying bio-ink, such as the biopolymer gel.

In at least some example embodiments of the device, the set comprises of components, such as: biological materials, biomaterials, metabolites, growth media, growth serums, growth factors, or other possible components of the bioprinted multi-component 3D object.

In at least some example embodiments of the device, the device may comprise two or more liquid dispensers arranged in an array, such as a linear array or any other suitable type of array. Each of the n carrier drops has a trajectory that passes by the two or more liquid dispensers configured to dispense drops towards the trajectory of the carrier drop in a synchronised manner, so that the carrier drop sequentially collides with the drops that aggregately comprise components from the subset and are dispensed by at least some of the two or more liquid dispensers in the array.

In at least some example embodiments, the device may comprise at least some of the liquid dispensers arranged in at least a first array and a second array, wherein the first array and the second array are configured to dispense drops towards a single stream of the carrier drops. In at least some example embodiments of the device, the device may comprise the first array is configured to dispense drops towards a first stream of carrier drops, and the second array is configured to dispense drops towards a second stream of carrier drops.

In at least some example embodiments of the device, the device may comprise a mechanisation configured to: move the support surface in at least one direction relative to the plurality of liquid dispensers, and/or move the plurality of liquid dispensers in at least one direction relative to the support surface, so that the n carrier drops in the stream land in the series of n successive XYZ coordinates on the support surface.

In at least some example embodiments of the device, the device may comprise a mixing chamber wherein the plurality of liquid dispensers, the support surface, and at least one environment regulator are positioned, and the at least one environment regulator is configured to regulate at least a temperature, a humidity, and a CO₂ concentration in the mixing chamber.

In at least some example embodiments of the device, the device may comprise a processing chamber wherein at least one processing element is installed and configured to regulate the a temperature, a humidity, a CO₂ concentration and light in the processing chamber, so that incubation of the multi-component 3D object, which may be located in the processing chamber, occurs.

In at least some example embodiments of the device, the device is used to bioprint the multi-component 3D object such as: an organ, an organ on a chip, a tissue, a scaffolds, or any other bioprintable multi-component 3D object.

In at least some example embodiments of the device, the device may comprise electric plates arranged perpendicularly to a path of at least one drop dispensed from at least one liquid dispenser, wherein the electric plates have holes through which the at least one drop passes, the at least one liquid dispenser is configured to provide an electric charge to the at least one drop, and the electric plates are configured so that the electric plates exert electric fields on the at least one drop in a synchronised manner, so that the at least one drop is manipulated with the electric fields exerted by the electric plates during the time in which the at least one drop passes through the electric fields exerted by the electric plates.

In at least some example embodiments of the method for bioprinting multi-component 3D objects (hereinafter: the method), the method includes dispensing and colliding drops by using the plurality of liquid dispensers, so that the stream of n carrier drops is dispensed and each of the n carrier drops collides and merges with at least drops that aggregately comprise one subset of components from a set during flight, and configuring a support surface so that the n carrier drops 10 land in a series of n successive XYZ coordinates, which represent the multi-component 3D object, so that the multi-component 3D object is bioprinted on the support surface 23.

In at least some example embodiments of the method, the method includes converting, by using a control unit, a 3D model of the multi-component 3D object into a series of n successive XYZ coordinates, and assigning, by using the control unit, one subset of components from the set to each of the XYZ coordinates in the series of n successive XYZ coordinates, so that the multi-component 3D object is represented by the series of n successive XYZ coordinates wherein each of the XYZ coordinates has one subset of components assigned.

In at least some example embodiments of the method, the method may include using any one of the example embodiments of the device according to the present disclosure.

At least some example embodiments of devices and methods according to the present disclosure relate to a device and a method for 3D printing multi-material 3D objects. Hereinafter some example embodiments of the devices and the methods that relate to the device and the method for 3D printing multi-material 3D objects are summarised. The technical problem relating to 3D printing of multi-material 3D objects is solved by the device for 3D printing multi-material 3D objects (hereinafter: the device) according to claim 50 and by the method for 3D printing multi-material 3D objects according to claim 62.

In at least some example embodiments of the device, the device comprises a plurality of liquid dispensers configured to dispense and collide drops, wherein a stream of n carrier drops is dispensed and each of the n carrier drops in the stream sequentially collides with at least drops that aggregately comprise one subset of materials from a set, and a support surface being configured so that the n carrier drops in the stream land in a series of n successive XYZ coordinates that represent the multi-material 3D object, so that the multi-material 3D object is 3D printed on the support surface. Each of the liquid dispensers in the plurality of liquid dispensers may comprise at least a container for holding liquids to dispense, a drop generating element positioned and configured to cause at least one drop to dispense, a nozzle through which the at least one drop is ejected from, a capillary which connects the container with the nozzle so that the liquid runs from the container through the capillary and out through the nozzle for the at least one drop to be dispensed, and a synchronisation means, which enable each of the liquid dispensers to operate in a synchronised manner with other liquid dispensers in the plurality of liquid dispensers. In some example embodiments of the device, a single container may be configured to supply liquids to two or more nozzles in the plurality of liquid dispensers. In some example embodiments of the device, two or more containers may be configured to supply liquids to a single nozzle in the plurality of liquid dispensers. Each of the liquid dispensers in the device may be being configured to dispense up to a hundred thousand (100000) drops per second. Each of the liquid dispensers in the device may be configured to dispense drops with a volume in a range between 1 picoliter to 100 nanoliters. The device may comprise fifty or more, or five hundred or more, or ten thousands or more liquid dispensers.

In at least some example embodiments of the device, the device comprises at least one liquid dispenser being configured to dispense the stream of the n carrier drops, wherein the stream comprises a first carrier drop, a second carrier drop, and at least a third carrier drop, and the plurality of liquid dispensers are configured so that the first carrier drop collides with at least drops that aggregately comprise a first subset of materials, the second carrier drop collides with at least drops that aggregately comprise a second subset of materials, the third carrier drop collides with at least drops that aggregately comprise a third subset materials from, and so on, until all of the n carrier drops in the stream collide with at least some drops that aggregately comprise at least some appropriate materials from the set, and all of the n carrier drops in the stream land in the series of n successive XYZ coordinates on the support surface.

In at least some example embodiments of the device, the n carrier drops include a binder material, such as a polymer, a photopolymer, or a water based binder, at the time of dispensing, or the n carrier drops include at least one type of material particles dispensed in the binder material.

In at least some example embodiments of the device, the set comprises of materials, such as: metal powders or granules, ceramic powders or granules, polymer particles, binder particles, or other materials of the 3D printed multi-material 3D object.

In at least some example embodiments of the device, the device may comprise two or more liquid dispensers arranged in an array, such as a linear array or any other suitable type of array, and/or the each of the n carrier drops has a trajectory that passes by the two or more liquid dispensers configured to dispense drops towards the trajectory of the carrier drop in a synchronised manner, so that the carrier drop sequentially collides with the drops that aggregately comprise materials from the subset and are dispensed by at least some of the two or more liquid dispensers in the array.

In at least some example embodiments of the device, the device may comprise at least some of the liquid dispensers arranged in at least a first array and a second array, wherein the first array and the second array are configured to dispense drops towards a single stream of the carrier drops. In at least some example embodiments of the device, the device may comprise the first array being configured to dispense drops towards a first stream of carrier drops, and the second array being configured to dispense drops towards a second stream of carrier drops.

In at least some example embodiments of the device, the device may comprise a mechanisation configured to: move the support surface in at least one direction relative to the plurality of liquid dispensers, and/or move the plurality of liquid dispensers in at least one direction relative to the support surface, so that the n carrier drops in the stream land in the series of n successive XYZ coordinates on the support surface.

In at least some example embodiments of the device, the device may comprise s a mixing chamber wherein the plurality of liquid dispensers, the support surface, and at least one environment regulator are positioned, and the environment regulator is configured to regulate at least a temperature and a humidity in the mixing chamber over time.

In at least some example embodiments of the device, the device may comprise the plurality of liquid dispensers comprising a temperature regulation system configured to regulate a temperature of the materials each liquid dispenser in the plurality of liquid dispensers before drops are dispensed, so that the drops are dispensed at a correct temperature.

In at least some example embodiments of the device, the device may comprise two or more liquid dispensers configured to dispense drops that contain a same material from the set, and the plurality of liquid dispensers is configured so that each of the n carrier drops collides with a different number of drops, which comprise the same material, a desired amount and density of the same material is contained in the each of the n carrier drops and the desired amount and density varies across the multi-material 3D object, which is 3D printed, so that a functionally graded multi-material 3D object is 3D printed, wherein a physical property of the functionally graded multi-material 3D object, such as density, varies in at least one direction in the functionally graded multi-material 3D object.

In at least some example embodiments of the device, the device may comprise electric plates arranged perpendicularly to a path of at least one drop dispensed from at least one liquid dispenser, wherein the electric plates have holes through which the at least one drop passes, the at least one liquid dispenser is configured to provide an electric charge to the at least one drop, and the electric plates are configured so that the electric plates exert electric fields on the at least one drop in a synchronised manner, so that the at least one drop is manipulated with the electric fields exerted by the electric plates during the time in which the at least one drop passes through the electric fields exerted by the electric plates.

In at least some example embodiments of the method for 3D printing multi-material 3D objects (hereinafter: the method), the method includes dispensing and colliding drops using the plurality comprising of liquid dispensers, so that a stream of n carrier drops is dispensed and each of the n carrier drops collides and merges with at least drops that aggregately comprise one subset of materials from a set during flight, and configuring a support surface so that the n carrier drops land in a series of n successive XYZ coordinates, which represent a multi-material 3D object, so that the multi-material 3D object is 3D printed on the support surface.

In at least some example embodiments of the method, the method includes includes converting, by using a control unit, a 3D model of the multi-material 3D object into the series of n successive XYZ coordinates, and assigning, by using the control unit, one subset of components from the set to each of the XYZ coordinates in the series of n successive XYZ coordinates, so that the multi-material 3D object is represented by the series of n successive XYZ coordinates wherein each of the XYZ coordinates has one subset of components assigned.

In at least some example embodiments of the method, the method for 3D printing multi-material 3D objects may include using any of the devices according to the present disclosure.

At least some example embodiments of devices and methods according to the present disclosure relate to a device and a method for manufacturing multi-substance tablets. Hereinafter some example embodiments of the devices and the methods that relate to the device and the method for manufacturing multi-substance tablets are summarised. The technical problem relating to manufacturing multi-substance tablets for oral or buccal delivery is solved by the device for manufacturing multi-substance tablets (hereinafter: the device) according to claim 65 and by the method for manufacturing multi-substance tablets (hereinafter: the method) according to claim 77.

In at least some example embodiments of the device, the device may comprise a plurality of liquid dispensers being configured to dispense and collide drops, wherein a stream of n carrier drops is dispensed and each of the n carrier drops in the stream sequentially collides with at least drops that aggregately comprise one subset of active pharmaceutical ingredients (hereinafter: APIs) and/or excipients from a set, and a support surface being configured so that the n carrier drops in the stream land in a series of n successive XYZ coordinates that represent a plurality of multi-substance tablets, so that the plurality multi-substance tablets is manufactured on the support surface. Each of the liquid dispensers in the plurality of liquid dispensers may comprise at least a container for holding liquids to dispense, a drop generating element positioned and configured to cause at least one drop to dispense, a nozzle through which the at least one drop is ejected from, a capillary which connects the container with the nozzle so that the liquid runs from the container through the capillary and out through the nozzle for the at least one drop to be dispensed, and a synchronisation means, which enable each of the liquid dispensers to operate in a synchronised manner with other liquid dispensers in the plurality of liquid dispensers. In some example embodiments of the device, a single container may be configured to supply liquids to two or more nozzles in the plurality of liquid dispensers. In some example embodiments of the device, two or more containers may be configured to supply liquids to a single nozzle in the plurality of liquid dispensers. Each of the liquid dispensers in the device may be being configured to dispense up to a hundred thousand (100000) drops per second. Each of the liquid dispensers in the device may be configured to dispense drops with a volume in a range between 1 picoliter to 1000 nanoliters. The device may comprise fifty or more, or five hundred or more, or ten thousands or more liquid dispensers.

In at least some example embodiments of the device, the device may comprise at least one liquid dispenser is configured to dispense the stream of the n carrier drops, wherein the stream comprises a first carrier drop, a second carrier drop, and at least a third carrier drop, and the plurality of liquid dispensers are configured so that the first carrier drop collides with at least drops that aggregately comprise a first subset of APIs and/or excipients, the second carrier drop collides with at least drops that aggregately comprise a second subset of APIs and/or excipients, the third carrier drop collides with at least drops that aggregately comprise a third subset of APIs and/or excipients from, and so on, until all of the n carrier drops in the stream collide with at least some drops that aggregately comprise at least some appropriate APIs and/or excipients from the set, and all of the n carrier drops in the stream land in the series of n successive XYZ coordinates on the support surface.

In at least some example embodiments of the device, the n carrier drops include a liquid ink carrier, such as: water, ethanol, propanol, or any other appropriate liquid ink carrier, or the n carrier drops include at least one API and the liquid ink carrier, or the n carrier drops include at least the liquid ink carrier and an excipient dispersion, so that rheological properties of the liquid ink carrier may be customised.

In at least some example embodiments of the device, the set may comprise APIs and/or excipients, such as: at least some APIs, polymers (such as but not limited to: hydroxypropyl methyl cellulose (HPMC), poly vinyl alcohol (PVA), Eudragit® derivates, polyvinylpyrroldone (PVP), polyethylene glycol (PEG)), surfactants (such as, but not limited to polysorbate 20—Tween 20®, polysorbate 80—Tween 80® or other), liquid ink carriers (such as: water, ethanol, propanol, or any combination of the liquid ink carriers listed herein), humectants (such as but not limited to: PEG, glycerol, or other), viscosity modifiers (such as but not limited to: glycerol, PEG, or other), solid excipient powder or granulate mixture, which may consist of various diluents, binding agents, disintegrants, lubricants, tablet coatings and films, colouring agents or other ingredients of the plurality of multi-substance tablets.

In at least some example embodiments of the device, the device may comprise two or more liquid dispensers arranged in an array, such as a linear array or any other suitable type of array comprising of two or more liquid dispensers arranged in an ordered manner, and/or the each of the n carrier drops has a trajectory that passes by the two or more liquid dispensers being configured to dispense drops towards the trajectory of the carrier drop in a synchronised manner, so that the carrier drop sequentially collides with the drops that aggregately comprise APIs and/or excipients from the subset and are dispensed by at least some of the two or more liquid dispensers in the array.

In at least some example embodiments of the device, the device may comprise the plurality of liquid dispensers being configured so that some of the n carrier drops in the stream collide with the drops that contain binder agents, and some other of the n carrier drops collide with the drops that contain solid excipients powders or granule mixtures suspended in highly volatile liquids, which evaporate before the carrier drops land, so that a high porosity of the plurality of multi-substance tablets is achieved, and the high porosity results in high disintegration.

In at least some example embodiments of the device, the device may comprise at least some liquid dispensers, which dispense drops that collide with any of the n carrier drops, being configured to dispense drops that contain agents that cause a sustained release of the APIs or a modified release of the APIs in the plurality of multi-substance tablets, so that a sustained release of the plurality of multi-substance tablets or a modified release of the plurality multi-substance tablets is achieved.

In at least some example embodiments of the device, the device may comprise a mechanisation being configured to move the support surface in at least one direction relative to the plurality of liquid dispensers, and/or move the plurality of liquid dispensers in at least one direction relative to the support surface, so that the n carrier drops in the stream land in the series of n successive XYZ coordinates on the support surface.

In at least some example embodiments of the device, the device may comprise a mixing chamber wherein the plurality of liquid dispensers, the support surface, and at least one environment regulator are positioned, and the environment regulator is configured to regulate at least a temperature and a humidity in the mixing chamber over time.

In at least some example embodiments of the device, the device may comprise two or more liquid dispensers arranged in an array, such as a linear array or a circular array. In at least some example embodiments of the device, the device may comprise at least some of the liquid dispensers arranged in at least a first array and a second array, wherein the first array and the second array are configured to dispense drops towards a single stream of the carrier drops. In at least some example embodiments of the device, the device may comprise the first array is configured to dispense drops towards a first stream of carrier drops, and the second array is configured to dispense drops towards a second stream of carrier drops.

In at least some example embodiments of the device, the device may comprise electric plates arranged perpendicularly to a path of at least one drop dispensed from at least one liquid dispenser, wherein the electric plates have holes through which the at least one drop passes, the at least one liquid dispenser is configured to provide an electric charge to the at least one drop, and the electric plates are configured so that the electric plates exert electric fields on the at least one drop in a synchronised manner, so that the at least one drop is manipulated with the electric fields exerted by the electric plates during the time in which the at least one drop passes through the electric fields exerted by the electric plates.

In at least some example embodiments of the method for manufacturing multi-substance tablets, the method comprises dispensing and colliding drops using the plurality of liquid dispensers, so that the stream of n carrier drops is dispensed and each of the n carrier drops collides and merges with at least drops that aggregately comprise one subset of APIs and/or excipients from the set during flight, and configuring the support surface so that the n carrier drops land in a series of n successive XYZ coordinates, which represent a plurality of multi-substance tablets, so that the plurality of multi-substance tablets is manufactured on the support surface.

In at least some example embodiments of the method for manufacturing multi-substance tablets (hereinafter: the method), the method includes converting, by using a control unit, a 3D model of a plurality of multi-substance tablets into a series of n successive XYZ coordinates, and assigning, by using the control unit, one subset of APIs and/or excipients from a set to each of the successive XYZ coordinates in the series of n successive XYZ coordinates, so that the plurality of multi-substance tablets is represented by the series of n successive XYZ coordinates wherein each of the successive XYZ coordinates has the subset of components assigned.

In at least some example embodiments of the method, the method may include using any of the devices according to the present disclosure.

At least some example embodiments of devices and methods according to the present disclosure relate to a device and a method for manufacturing micro and/or nano therapeutical particles for parerental, respiratory or nasal delivery. Hereinafter some example embodiments of the devices and the methods that relate to the device and the method for manufacturing micro and/or nano for parerental, respiratory or nasal delivery are summarised. The technical problem relating to manufacturing micro and/or nano therapeutical particles is solved by the device for manufacturing micro and/or nano therapeutical particles for parerental, respiratory or nasal delivery (hereinafter: the device) according to claim 80 and by the method for manufacturing micro and/or nano therapeutical particles for parerental, respiratory or nasal delivery (hereinafter: the method) according to claim 94.

In at least some example embodiments of the device, the device may comprise a plurality of liquid dispensers being configured to dispense and collide drops, wherein a stream of n carrier drops is dispensed and each of the n carrier drops in the stream sequentially collides with at least drops that aggregately comprise one subset of active pharmaceutical ingredients (hereinafter: APIs) and/or excipients from a set; and a support surface being configured so that the n carrier drops in the stream land in a series of select XY coordinates, so that the micro and/or nano sized therapeutical particles are manufactured in the select XY coordinates on a support surface. Each of the liquid dispensers in the plurality of liquid dispensers may comprise at least a container for holding liquids to dispense, a drop generating element positioned and configured to cause at least one drop to dispense, a nozzle through which the at least one drop is ejected from, a capillary which connects the container with the nozzle so that the liquid runs from the container through the capillary and out through the nozzle for the at least one drop to be dispensed, and a synchronisation means, which enable each of the liquid dispensers to operate in a synchronised manner with other liquid dispensers in the plurality of liquid dispensers. In some example embodiments of the device, a single container may be configured to supply liquids to two or more nozzles in the plurality of liquid dispensers. In some example embodiments of the device, two or more containers may be configured to supply liquids to a single nozzle in the plurality of liquid dispensers. Each of the liquid dispensers in the device may be being configured to dispense up to a hundred thousand (100000) drops per second. Each of the liquid dispensers in the device may be configured to dispense drops with a volume in a range between 1 picoliter to 100 nanoliters. The device may comprise fifty or more, or five hundred or more, or ten thousands or more liquid dispensers.

In at least some example embodiments of the device, the device may comprise at least one liquid dispenser being configured to dispense the stream of the n carrier drops, wherein the stream comprises a first carrier drop, a second carrier drop, and at least a third carrier drop, and the plurality of liquid dispensers are configured so that the first carrier drop collides with at least drops that aggregately comprise a first subset of APIs and/or excipients, the second carrier drop collides with at least drops that aggregately comprise a second subset of APIs and/or excipients, the third carrier drop collides with at least drops that aggregately comprise a third subset of APIs and/or excipients from, and so on, until all of the n carrier drops in the stream collide with at least some drops that aggregately comprise at least some appropriate APIs and/or excipients from the set, and all of the n carrier drops in the stream land in the series of select XY coordinates on the support surface.

In at least some example embodiments of the device, the n carrier drops include an excipient dispersion that is capable of carrying substances of the micro and/or nano sized therapeutical particles, or the n carrier drops include the excipient dispersion and at least one API, or the n carrier drops comprise anti-solvent liquids, such as: water, tert-butanol, ethanol, so that substances contained in drops that collide with the carrier drops, do not solve in the n carrier drops comprising the anti-solvent liquids.

In at least some example embodiments of the device, the set comprises of APIs and/or excipients, such as: at least some APIs (therapeutic peptides or biologically-active small molecules), biodegradable carrier polymer for sustained-release (such as: poly lactide (PLA), poly (lactic-co-glycolic) acid (PLGA)), liquid ink carriers (such as: water, dimethyl sulfoxide (DMSO), ethyl acetate), anti-solvent liquids (such as: water, tert-butanol, ethanol) or other substances, which constitute the micro and/or nano sized therapeutical particles.

In at least some example embodiments of the device, the device may comprise two or more liquid dispensers arranged in an array, such as a linear array or any other suitable type of array, and/or the each of the n carrier drops has a trajectory that passes by the two or more liquid dispensers configured to dispense drops towards the trajectory of the carrier drop in a synchronised manner, so that the carrier drop sequentially collides with the drops that aggregately comprise APIs and/or excipients from the subset and may be dispensed by at least some of the two or more liquid dispensers in the array.

In at least some example embodiments of the device, the device may comprise a freeze drying element mounted on the support surface, wherein the freeze-drying element is configured to freeze dry the carrier drops immediately after the carrier drops land on the support surface, so that spherically shaped micro and/or nano sized therapeutical particles may be produced.

In at least some example embodiments of the device, the select XY coordinates correlate with internal part of vials or cartridges (for inhalation devices) that may be positioned on the support surface, and a Z coordinate may relate to an amount of micro and/or nano sized therapeutical particles to be deposited in the vials or cartridges (for inhalation devices) that may be positioned at the select XY coordinates on the support surface.

In at least some example embodiments of the device, the device may comprise a mechanisation configured to move the support surface in at least one direction relative to the plurality of liquid dispensers, and/or move the plurality of liquid dispensers in at least one direction relative to the support surface, so that the n carrier drops in the stream land in at least the select XY coordinates on the support surface.

In at least some example embodiments of the device, the device may comprise a mixing chamber wherein the plurality of liquid dispensers, the support surface, and at least one environment regulator are positioned, and the environment regulator is configured to regulate at least a temperature and a humidity in the mixing chamber over time.

In at least some example embodiments of the device, the environment regulator is configured to provide the temperature and the humidity in the mixing chamber so that the carrier drops evaporate or spray dry before landing in at least the select XY coordinates, so that the spherically shaped nano and/or micro sized therapeutical particles are produced.

In at least some example embodiments of the device, the device may comprise two or more liquid dispensers arranged in an array, such as a linear array or a circular array. In at least some example embodiments of the device, the device may comprise at least some of the liquid dispensers arranged in at least a first array and a second array, wherein the first array and the second array are configured to dispense drops towards a single stream of the carrier drops. In at least some example embodiments of the device, the device may comprise the first array is configured to dispense drops towards a first stream of carrier drops, and the second array is configured to dispense drops towards a second stream of carrier drops.

In at least some example embodiments of the device, the device may comprise electric plates arranged perpendicularly to a path of at least one drop dispensed from at least one liquid dispenser, wherein the electric plates have holes through which the at least one drop passes, the at least one liquid dispenser is configured to provide an electric charge to the at least one drop, and the electric plates are configured so that the electric plates exert electric fields on the at least one drop in a synchronised manner, so that the at least one drop is manipulated with the electric fields exerted by the electric plates during the time in which the at least one drop passes through the electric fields exerted by the electric plates.

In at least some example embodiments of the device, the device may comprise a processing chamber wherein at least one processing element is installed, and the at least one processing element is configured to remove the anti-solvent liquids from each of n carrier drops located in the processing chamber that includes the anti-solvent liquids.

In at least some example embodiments of the method for manufacturing micro and/or nano sized therapeutical particles for parerental, respiratory or nasal delivery (hereinafter: the method), the method includes dispensing and colliding drops using a plurality of liquid dispensers, so that a stream of n carrier drops is dispensed and each of the n carrier drops collides and merges with at least drops that aggregately comprise one subset of APIs and/or excipients from a set during flight, and configuring a support surface so that the n carrier drops land in at least a series of select XY coordinates, so that micro and/or nano sized therapeutical particles are manufactured in the series of select XY coordinates on the support surface.

In at least some example embodiments of the method, the method may include using any of the devices according to the present disclosure.

At least some example embodiments of devices and methods according to the present disclosure relate to a device and a method for applying active pharmaceutical ingredients (hereinafter: APIs) and/or excipients on pre-manufactured placebos. Hereinafter some example embodiments of the devices and the methods that relate to the device and the method for applying APIs and/or excipients on pre-manufactured placebos are summarised. The technical problem relating to applying APIs and/or excipients on pre-manufactured placebos is solved by the device for applying APIs and/or excipients on pre-manufactured placebos (hereinafter: the device) according to claim 96 and by the method for applying APIs and/or excipients on pre-manufactured placebos (hereinafter: the method) according to claim 109.

In at least some example embodiments of the device, the device may comprise a plurality of liquid dispensers being configured to dispense drops, wherein a stream of carrier drops is dispensed, and each of the carrier drops in the stream sequentially collides with a pre-determined number of the drops that aggregately comprise one subset of APIs and/or excipients selected from a set of APIs and/or excipients, so that a pre-determined amount and combination of APIs and/or excipients is contained in each of the carrier drops in the stream during flight; and a support surface being configured so that the each of the carrier drops in the stream lands on one of select XY coordinates on a plurality of pre-manufactured placebos positioned on the support surface 23. Each of the liquid dispensers in the plurality of liquid dispensers may comprise at least a container for holding liquids to dispense, a drop generating element positioned and configured to cause at least one drop to dispense, a nozzle through which the at least one drop is ejected from, a capillary which connects the container with the nozzle so that the liquid runs from the container through the capillary and out through the nozzle for the at least one drop to be dispensed, and a synchronisation means, which enable each of the liquid dispensers to operate in a synchronised manner with other liquid dispensers in the plurality of liquid dispensers. In some example embodiments of the device, a single container may be configured to supply liquids to two or more nozzles in the plurality of liquid dispensers. In some example embodiments of the device, two or more containers may be configured to supply liquids to a single nozzle in the plurality of liquid dispensers. Each of the liquid dispensers in the device may be being configured to dispense up to a hundred thousand (100000) drops per second. Each of the liquid dispensers in the device may be configured to dispense drops with a volume in a range between 1 picoliter to 100 nanoliters. The device may comprise fifty or more, or five hundred or more, or ten thousands or more liquid dispensers.

In at least some example embodiments of the device, the device may comprise at least one liquid dispenser being configured to dispense the stream of carrier drops, and/or the stream of carrier drop has a trajectory that passes by two or more liquid dispensers configured to dispense drops towards the trajectory of the stream of carriers drop in a synchronised manner, so that each of the carrier drop in the stream sequentially collides with at least drops that aggregately comprise one pre-determined amount and combination of APIs and/or excipients from the set, and are dispensed by at least some of the two or more liquid dispensers.

In at least some example embodiments of the device, the stream comprises a first carrier drop, a second carrier drop, and at least a third carrier drop, and the plurality of liquid dispensers are configured so that the first carrier drop collides with drops that aggregately comprise a first pre-determined amount and combination of APIs and/or excipients, the second carrier drop collides with drops that aggregately comprise a second pre-determined amount and combination of APIs and/or excipients, the third carrier drop collides with drops that aggregately comprise a third pre-determined amount and combination of APIs and/or excipients, and so on, until all of the carrier drops in the stream collide with at least some drops that aggregately comprise any desired pre-determined amount and combination of APIs and/or excipients from the set, all of the carrier drops in the stream land on the plurality of pre-manufactured placebos positioned on the support surface, and a variable dosing of APIs and/or excipients may be achieved across the plurality of pre-manufactured placebos.

In at least some example embodiments of the device, the device may comprise two or more liquid dispensers arranged in an array, such as a linear array or a circular array. In at least some example embodiments of the device, the device may comprise at least some of the liquid dispensers arranged in at least a first array and a second array, wherein the first array and the second array are configured to dispense drops towards a single stream of the carrier drops. In at least some example embodiments of the device, the device may comprise the first array is configured to dispense drops towards a first stream of carrier drops, and the second array is configured to dispense drops towards a second stream of carrier drops.

In at least some example embodiments of the device, the support surface is positioned so that two or more carrier drops from the stream land at a single select XY coordinate whereat one pre-manufactured placebo is located, wherein the pre-determined amount of APIs and/or excipients is contained in the two or more carrier drops.

In at least some example embodiments of the device, the device may comprise the plurality of liquid dispensers being configured so that each API from the set is suspended in a liquid in a single container that supplies the liquid to two or more liquid dispensers in the plurality of liquid dispensers, and each excipient from the set is suspended in another liquid in another container, which supplies the another liquid to another two or more liquid dispensers in the plurality of liquid dispensers. The carrier drops may include high loaded APIs at the time of dispensing.

In at least some example embodiments of the device, the device comprises a mechanisation configured to move support surface in at least one direction relative to the plurality of liquid dispensers, and/or move the plurality of liquid dispensers in at least one direction relative to support surface.

In at least some example embodiments of the device, the device may comprise a mixing chamber, which is a partially or a fully closed compartment of the device wherein the plurality of liquid dispensers and the support surface are positioned, and the mixing chamber is the fully closed compartment during the time in which the drops are in-flight.

In at least some example embodiments of the device, the set comprises of APIS and/or excipients, such as: at least some APIs, polymers (such as: hydroxypropyl methyl cellulose (HPMC), poly vinyl alcohol (PVA), Eudragit® derivates, polyvinylpyrroldone (PVP), polyethylene glycol (PEG)), surfactants (such as: Polysorbate 20—Tween 20® or other), liquid ink carriers (such as: water, ethanol, propanol, or any combination of liquid ink carriers), humectants (such as: PEG, glycerol, or other), viscosity modifiers (such as but not limited to: glycerol, PEG, or other) or other ingredients, which are applied on to the pre-manufactured placebos.

In at least some example embodiments of the device, the plurality of pre-manufactured placebos positioned on the support surface, are pre-manufactured placebos, such as: oral tablets, capsules, orodispensible films, films in capsules, mucoadhesive films or other types of pre-manufactured placebos.

In at least some example embodiments of the device, the device may comprise electric plates arranged perpendicularly to a path of at least one drop dispensed from at least one liquid dispenser, wherein the electric plates have holes through which the at least one drop passes, the at least one liquid dispenser is configured to provide an electric charge to the at least one drop, and the electric plates are configured so that the electric plates exert electric fields on the at least one drop in a synchronised manner, so that the at least one drop is manipulated with the electric fields exerted by the electric plates during the time in which the at least one drop passes through the electric fields exerted by the electric plates In at least some example embodiments of the method for applying active pharmaceutical ingredients and/or excipients on pre-manufactured placebos (hereinafter: the method), the method comprises dispensing, by using a plurality of liquid dispensers, a stream of carrier drops and at least a pre-determined number of drops that aggregately comprise one subset of APIs and/or excipients selected from a set, colliding each of the carrier drops in the stream with drops that aggregately comprise the one subset of APIs and/or excipients so that a pre-determined amount and combination of APIs and/or excipients is contained in each of the carrier drops during flight, and configuring a support surface so that the each of the carrier drops lands on one of the pre-manufactured placebos, which are positioned on the support surface at select XY coordinates.

In at least some example embodiments of the method, the method may include using any of the devices according to the present disclosure.

At least some example embodiments of devices and methods according to the present disclosure relate to a device and a method for applying active pharmaceutical ingredients (hereinafter: APIs) and/or excipients on medical devices. Hereinafter some example embodiments of the devices and the methods that relate to the device and the method for applying APIs and/or excipients on medical devices are summarised. The technical problem relating to applying APIs and/or excipients on medical devices is solved by the device for applying APIs and/or excipients on medical devices (hereinafter: the device) according to claim 111 and by the method applying for applying APIs and/or excipients on medical devices (hereinafter: the method) according to claim 125.

In at least some example embodiments of the device, the device may comprise a plurality of liquid dispensers being configured to dispense drops, wherein a stream of carrier drops is dispensed, and each of the carrier drops in the stream sequentially collides with a pre-determined number of the drops that aggregately comprise one subset of APIs and/or excipients selected from a set, so that a pre-determined amount and combination of APIs and/or excipients is contained in each of the carrier drops in the stream during flight; and a support surface being configured so that the each of the carrier drops in the stream lands on one select XYZ coordinate whereat a medical device is positioned on the support surface. Each of the liquid dispensers in the plurality of liquid dispensers may comprise at least a container for holding liquids to dispense, a drop generating element positioned and configured to cause at least one drop to dispense, a nozzle through which the at least one drop is ejected from, a capillary which connects the container with the nozzle so that the liquid runs from the container through the capillary and out through the nozzle for the at least one drop to be dispensed, and a synchronisation means, which enable each of the liquid dispensers to operate in a synchronised manner with other liquid dispensers in the plurality of liquid dispensers. In some example embodiments of the device, a single container may be configured to supply liquids to two or more nozzles in the plurality of liquid dispensers. In some example embodiments of the device, two or more containers may be configured to supply liquids to a single nozzle in the plurality of liquid dispensers. Each of the liquid dispensers in the device may be being configured to dispense up to a hundred thousand (100000) drops per second. Each of the liquid dispensers in the device may be configured to dispense drops with a volume in a range between 1 picoliter to 100 nanoliters. The device may comprise fifty or more, or five hundred or more, or ten thousands or more liquid dispensers.

In at least some example embodiments of the device, the device may comprise at least one liquid dispenser being configured to dispense the stream of carrier drops, and/or the stream of carrier drop has a trajectory that passes by two or more liquid dispensers configured to dispense drops towards the trajectory of the stream of carriers drop in a synchronised manner, so that each of the carrier drop in the stream sequentially collides with at least drops that aggregately comprise one pre-determined amount and combination of APIs and/or excipients from the set, and may be dispensed by at least some of the two or more liquid dispensers.

In at least some example embodiments of the device, the stream comprises a first carrier drop, a second carrier drop, and at least a third carrier drop, and the plurality of liquid dispensers are configured so that the first carrier drop collides with drops that aggregately comprise a first pre-determined amount and combination of APIs and/or excipients, the second carrier drop collides with drops that aggregately comprise a second pre-determined amount and combination of APIs and/or excipients, the third carrier drop collides with drops that aggregately comprise a third pre-determined amount and combination of APIs and/or excipients, and so on, until all of the carrier drops in the stream collide with at least some drops that aggregately comprise any desired pre-determined amount and combination of APIs and/or excipients from the set, all of the carrier drops in the stream land on the medical device positioned on the support surface, and a variable dosing of APIs and/or excipients may be achieved across the select XY coordinates on the medical device.

In at least some example embodiments of the device, the device may comprise two or more liquid dispensers arranged in an array, such as a linear array or a circular array. In at least some example embodiments of the device, the device may comprise at least some of the liquid dispensers arranged in at least a first array and a second array, wherein the first array and the second array are configured to dispense drops towards a single stream of the carrier drops. In at least some example embodiments of the device, the first array may be configured to dispense drops towards a first stream of carrier drops, and the second array may be configured to dispense drops towards a second stream of carrier drops.

In at least some example embodiments of the device, the support surface may be positioned so that two or more carrier drops from the stream land at a single select XY coordinate whereat one pre-manufactured placebo is located, wherein the pre-determined amount of APIs and/or excipients is contained in the two or more carrier drops. In at least some example embodiments of the device, the carrier drops may include high loaded APIs at the time of dispensing.

In at least some example embodiments of the device, the device may comprise the plurality of liquid dispensers being configured so that each API from the set is suspended in a liquid in a single container that supplies the liquid to two or more liquid dispensers in the plurality of liquid dispensers, and each excipient from the set is suspended in another liquid in another container, which supplies the another liquid to another two or more liquid dispensers in the plurality of liquid dispensers.

In at least some example embodiments of the device, the device may comprise a mechanisation configured to move the support surface in at least one direction relative to the plurality of liquid dispensers, and/or move the plurality of liquid dispensers in at least one direction relative to the support surface.

In at least some example embodiments of the device, the device may comprise a mixing chamber, which is a partially or a fully closed compartment of the device wherein the plurality of liquid dispensers and the support surface are positioned, and the mixing chamber is the fully closed compartment during the time in which the drops are in-flight.

In at least some example embodiments of the device, drops that collide with the carrier drop comprise APIs and/or excipients, such as: APIs, polymers (such as: hydroxypropyl methyl cellulose (HPMC), poly vinyl alcohol (PVA), Eudragit® derivates, polyvinylpyrroldone (PVP), polyethylene glycol (PEG), Soluplus®), surfactants (such as: polysorbate 20—Tween 20®, polysorbate 80—Tween 80® or other), liquid ink carriers (such as: water, ethanol, propanol, dimethyl sulfoxide (DMSO) or any combination of liquid ink carriers), humectants (such as: PEG, glycerol, or other), viscosity modifiers (such as: glycerol, PEG, or other), or other ingredients to apply to the medical device or the micro-needles.

In at least some example embodiments of the device, multiple thin film layers of carrier drops are deposited on the medical device and the variable dosing of APIs and/or excipients is achieved across the multiple thin film layers applied on the medical device.

In at least some example embodiments of the device, the medical device positioned on the support surface is any medical device, such as: at least one implant, a micro-needle patch or any other medical device.

In at least some example embodiments of the device, the device may comprise electric plates arranged perpendicularly to a path of at least one drop dispensed from at least one liquid dispenser, wherein the electric plates have holes through which the at least one drop passes, the at least one liquid dispenser is configured to provide an electric charge to the at least one drop, and the electric plates are configured so that the electric plates exert electric fields on the at least one drop in a synchronised manner, so that the at least one drop is manipulated with the electric fields exerted by the electric plates during the time in which the at least one drop passes through the electric fields exerted by the electric plates

In at least some example embodiments of the method for applying active pharmaceutical ingredients (hereinafter: APIs) and/or excipients on medical devices (hereinafter: the method), the method includes dispensing, by using a plurality of liquid dispensers, a stream of carrier drops and at least a pre-determined number of drops that aggregately comprise one subset of APIs and/or excipients selected from a set, colliding each of the carrier drops in the stream with drops that aggregately comprise the one subset of APIs and/or excipients so that a pre-determined amount and combination of APIs and/or excipients is contained in each of the carrier drops during flight, and configuring a support surface so that the each of the carrier drops lands on one select XY coordinate on the support surface whereat a medical device is positioned.

In at least some example embodiments of the method, the method may include using any of the devices according to the present disclosure.

At least some example embodiments of devices and methods according to the present disclosure relate to a device and a method for assembly of nucleic acids for pharmaceutical, phytopharmaceutical, or veterinary use. Hereinafter some example embodiments of the device and the method that relate to the device and the method for assembly of nucleic acids for pharmaceutical, phytopharmaceutical, or veterinary use are summarised. The technical problem relating to assembly of nucleic acids for pharmaceutical, phytopharmaceutical, or veterinary use is solved by the device for assembly of nucleic acids for pharmaceutical, phytopharmaceutical, or veterinary use (hereinafter: the device) according to claim 127 and by the method for assembly of nucleic acids for pharmaceutical, phytopharmaceutical, or veterinary use (hereinafter: the method) according to claim 140.

In at least some example embodiments of the device, the device may comprise a plurality of liquid dispensers being configured to dispense drops, wherein at least a carrier drop sequentially collides with at least drops that aggregately comprise a subset of nucleic acids from a set, so that the subset of nucleic acids is located in the carrier drop, wherein the subset of nucleic acids comprises all components to produce final nucleic acid molecules, and a support surface configured to collect the carrier drop. Each of the liquid dispensers in the plurality of liquid dispensers may comprise at least a container for holding liquids to dispense, a drop generating element positioned and configured to cause at least one drop to dispense, a nozzle through which the at least one drop is ejected from, a capillary which connects the container with the nozzle so that the liquid runs from the container through the capillary and out through the nozzle for the at least one drop to be dispensed, and a synchronisation means, which enable each of the liquid dispensers to operate in a synchronised manner with other liquid dispensers in the plurality of liquid dispensers. In some example embodiments of the device, a single container may be configured to supply liquids to two or more nozzles in the plurality of liquid dispensers. In some example embodiments of the device, two or more containers may be configured to supply liquids to a single nozzle in the plurality of liquid dispensers. Each of the liquid dispensers in the device may be being configured to dispense up to a hundred thousand (100000) drops per second. Each of the liquid dispensers in the device may be configured to dispense drops with a volume in a range between 1 picoliter to 100 nanoliters. The device may comprise fifty or more, or five hundred or more, or ten thousands or more liquid dispensers.

At least one liquid dispenser in the device is configured to dispense the carrier drop. The carrier drop may have a trajectory that passes by two or more liquid dispensers configured to dispense drops towards the trajectory of the carrier drop in a synchronised manner, so that the carrier drop sequentially collides with at least all of the drops that contain components from the subset, and are dispensed by at least some of the two or more liquid dispensers.

In at least some example embodiments of the device, the device may comprise at least one liquid dispenser is configured to dispense a stream of carrier drops, wherein the stream comprises a first carrier drop, a second carrier drop, and at least a third carrier drop, and the plurality of liquid dispensers are configured so that the first carrier drop collides with at least drops that aggregately comprise components from a first subset, the second carrier drop collides with at least drops that aggregately comprise components from a second subset, the third carrier drop collides with at least drops that aggregately comprise components from a third subset, and so on, until all of the carrier drops in the stream collide with at least some drops that aggregately comprise at least some components from the set, and all of the carrier drops in the stream land on the support surface.

In at least some example embodiments of the device, the device may comprise two or more liquid dispensers arranged in an array, such as a linear array or a circular array. In at least some example embodiments of the device, the device may comprise at least some of the liquid dispensers arranged in at least a first array and a second array, wherein the first array and the second array are configured to dispense drops towards a single stream of the carrier drops. In at least some example embodiments of the device, the device may comprise the first array is configured to dispense drops towards a first stream of carrier drops, and the second array is configured to dispense drops towards a second stream of carrier drops.

In at least some example embodiments of the device, the device may comprise each of the liquid dispensers in the plurality of liquid dispensers being configured to dispense up to a hundred thousand (100000) drops per second. In at least some example embodiments of the device, the device may comprise each of the liquid dispensers in the plurality of liquid dispensers being configured to dispense drops with a volume in a range between 1 picoliter to 100 nanoliters. In at least some example embodiments of the device, the device may comprise the plurality of liquid dispensers comprising of fifty or more, or five hundred or more, or ten thousands or more liquid dispensers.

In at least some example embodiments of the device, the support surface is made of a bendable material or a non-bendable material, and/or is detachable or non-detachable relative to the device, and/or is a container filled with a liquid in which the carrier drops land, and/or is a flat surface.

In at least some example embodiments of the device, the device may comprise the plurality of liquid dispensers being configured to dispense drops of a first liquid, and the support surface is filled or covered with a second liquid, and the first liquid and the second liquid are immiscible.

In at least some example embodiments of the device, the device may comprise electric plates arranged perpendicularly to a path of at least one drop dispensed from at least one liquid dispenser, wherein the electric plates have holes through which the at least one drop passes, the at least one liquid dispenser is configured to provide an electric charge to the at least one drop, and the electric plates are configured so that the electric plates exert electric fields on the at least one drop in a synchronised manner, so that the at least one drop is manipulated with the electric fields exerted by the electric plates during the time in which the at least one drop passes through the electric fields exerted by the electric plates.

In at least some example embodiments of the device, the device may comprise a mixing chamber, which is a partially or a fully closed compartment of the device wherein the plurality of liquid dispensers and the support surface are positioned, and the mixing chamber is the fully closed compartment during the time in which the drops are in-flight.

In at least some example embodiments of the device, the carrier drop includes binding reagents, which enable an assembly and binding together of the components located in the carrier drop, wherein the binding reagents are included in the carrier drop or provided by a drop that collides and merges with the carrier drop during flight.

In at least some example embodiments of the device, the device may comprise a processing chamber having at least one temperature regulator configured to change a temperature in the processing chamber over time so that a sequence of temperatures is achieved over time in the processing chamber, and the sequence of temperatures causes a biochemical reaction to occur between the components and binding reagents located in each carrier drop positioned in the processing chamber, wherein the biochemical reaction causes the components located in each of the carrier drops to assemble and bind together to form the final nucleic acid molecules, wherein the biochemical reaction is either a Polymerase Chain Reaction, or a Sticky End Ligation, or a Cell Free Cloning, or a BioBricks Assembly, or a Gibson Assembly, or a HiFi Assembly, and wherein at least some of the biochemical reactions include multiplication of the final nucleic acid molecules.

In at least some example embodiments of the device, the device may comprise a mechanisation configured to move the support surface in at least one direction relative to the plurality of liquid dispensers, and/or move the plurality of liquid dispensers in at least one direction relative to the support surface, and/or move the support surface from the mixing chamber to the processing chamber.

In at least some example embodiments of the device, the device may comprise each of the liquid dispensers in the plurality of liquid dispensers comprising a container for holding liquids to dispense, a drop generating element positioned and configured to cause at least one drop to dispense, a nozzle through which the at least one drop is ejected from, a capillary which connects the container with the nozzle so that the liquid runs/flows from the container through the capillary and out through the nozzle for the at least one drop to be dispensed, and a synchronisation means, which enable each of the liquid dispensers to operate in a synchronised manner with other liquid dispensers in the plurality of liquid dispensers; and/or a single container being configured to supply liquids to two or more nozzles in the plurality of liquid dispensers; and/or two or more containers being configured to supply liquids to a single nozzle in the plurality of liquid dispensers.

In at least some example embodiments of the method for assembly of nucleic acids for pharmaceutical, phytopharmaceutical, or veterinary use (hereinafter: the method), the method comprises dispensing, by use of a plurality of liquid dispensers, at least one carrier drop and at least drops that aggregately comprise a subset of components from a set of nucleic acid components, wherein the carrier drop sequentially collides with at least drops that aggregately comprise the subset of components, so that the subset of components is located in the carrier drop during flight, and collecting the at least one carrier drop by using a support surface.

In at least some example embodiments of the method, the method may include using any of the devices according to the present disclosure.

All of the drops are dispensed.

Terms such as ‘a’, ‘an’, and ‘the’ are not intended to refer to only a singular entity but include the general class of which specific example may be used for illustration. The terminology is herein used to describe specific embodiments of the invention, but their usage does not delimit the invention, except as outlined in the claims.

### BRIEF DESCRIPTION OF DRAWINGS

FIG. 1 schematically depicts a front view of an example embodiment of the device.

FIG. 2 schematically depicts a front view of an example embodiment of the device comprising two arrays of liquid dispensers.

FIG. 3 schematically depicts a front view of an example embodiment of the device comprising a processing chamber.

FIG. 4 schematically depicts a front view of an example embodiment of the device comprising electric plates.

FIG. 5A schematically depicts a top view of three linear arrays of liquid dispensers.

FIG. 5B schematically depicts a top view of four linear arrays of liquid dispensers.

FIG. 6 schematically depicts main steps of the method.

### DETAILED DESCRIPTION

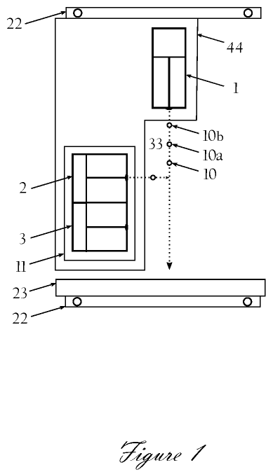

At least some example embodiments according to the present disclosure relate to a device and a method for mixing liquids. Hereinafter at least some example embodiments of devices and methods that relate to the device and the method for mixing liquids are described in more detail. In at least some example embodiments, the device for mixing liquids (hereinafter: the device) is configured to produce liquid mixtures in a combinatorial manner, by using the method for mixing liquids (hereinafter: the method) according to the present disclosure. In at least some example embodiments, the device and the method are used to produce liquid mixtures from a set of liquids that comprises a plurality of liquids. A plurality of liquid mixtures may be produced using the device and the method, any each liquid mixture may comprise two or more liquids from the set. The set of liquids is held in a plurality of containers in at least some of the liquid dispensers in a plurality 44 of liquid dispensers, which comprises at least liquid dispensers 1-3 (FIG. 1) in the device. Each of the liquids from the set is held in a separate container in at least one liquid dispenser in the plurality 44 of liquid dispensers in the device.

In at least one preferred example embodiment of the device, such as depicted in FIG. 1, the device comprises the plurality 44 of liquid dispensers 1-3 configured to dispense drops, wherein at least a carrier drop 10 is dispensed and sequentially collides and merges with drops dispensed from a sub-plurality of liquid dispensers, so that liquids from the sub-plurality of liquid dispensers are located in the carrier drop 10 during flight, and a support surface 23 being positioned so that the carrier drop 10 lands on the support surface 23. In at least some example embodiments of the device, each of the liquid dispensers in the plurality 44 comprising of at least the liquid dispensers 1-3 includes at least a container for holding liquids to dispense, a drop generating element positioned and configured to cause at least one drop to dispense, a nozzle through which the at least one drop is ejected from, a capillary which connects the container with the nozzle so that the liquid runs from the container through the capillary and out through the nozzle for the at least one drop to be dispensed, and synchronisation means, which enable each of the liquid dispensers to operate in a synchronised manner with other liquid dispensers in the plurality 44 of liquid dispensers. In some example embodiments, a single container is configured to supply liquids to two or more nozzles in the plurality 44 of liquid dispensers. In some example embodiments, two or more containers are configured to supply liquids to a single nozzle in the plurality 44 of liquid dispensers. At least one liquid dispenser, such as the liquid dispenser 1 in FIG. 1, may be configured to dispense a stream 33 of carrier drops 10 (10a, 10b . . . ), wherein the stream 33 comprises a first carrier drop 10, a second carrier drop 10a, and at least a third carrier drop 10b, and the plurality 44 of liquid dispensers may be configured so that the first carrier drop 10 collides with drops from a first sub-plurality of liquid dispensers, the second carrier drop 10a collides with drops from a second sub-plurality of liquid dispensers, the third carrier drop 10b collides with drops from a third sub-plurality of liquid dispensers, and so on, until all of the carrier drops 10 (10a, 10b . . . ) in the stream 33 collide with at least some drops dispensed from at least some liquid dispensers in the plurality 44 of liquid dispensers, and all of the carrier drops 10 (10a, 10b . . . ) in the stream 33 land on the support surface 23. The first sub-plurality of liquid dispensers may include, for example, five (5) liquid dispensers in the plurality 44 of thirty (30) liquid dispensers in the device, and all five (5) drops dispensed from the first sub-plurality of the five (5) liquid dispensers may collide and merge with the first carrier drop 10 in the stream 33 during flight. The second sub-plurality may include seven (7) liquid dispensers in the plurality 44 of (in this given example) thirty (30) liquid dispensers in device, and all seven (7) drops dispensed from the sub-plurality of seven (7) liquid dispensers collide and merge with the second carrier drop 10a in the stream 33 during flight, and so on. Collisions occur between each of the carrier drops 10 (10a, 10b . . . ) in the stream 33 and all drops that carry liquids from any one desired sub-plurality of liquids from the set. It is understood that any appropriate combinations of liquids may be included in the set, or in any sub-plurality, so that custom and highly complex liquid mixtures (or solid compounds) may be produced using the device and the method. In a preferred example embodiment of the device, the device comprises more than five hundred (500) liquid dispensers in the plurality 44. Liquids in the set may carry materials and/or components dispensed at a known concentration. All of the drops are dispensed. The stream 33 may be continuous or discontinuous.

In at least some example embodiments of the device, the device comprises a user interface configured to provide user data, which may include the method settings, the device settings, and any other relevant user data. In at least some example embodiments of the device, the device comprises a control unit 1000, which may be configured to create a combinatorial model of the liquid mixtures (or solid compounds) to be produced at select XY coordinates on the support surface 23 whereat the liquid mixtures located in the carrier drops 10 (10a, 10b . . . ) may land. The select XY coordinates comprises all select XY coordinates, which represent the locations whereat the liquid mixtures produced in the carrier drops 10 (10a, 10b . . . ) may be deposited. Each of the select XY coordinates includes an X and a Y coordinate relative to the support surface 23. In at least some example embodiments, a Z coordinate may be a part of the select XY coordinate, and the Z coordinate may correspond to an amount of the liquid mixture to be deposited at the XY coordinate. In one example embodiment, the X and the Y coordinates are located on a plane on the support surface 23, and the Z coordinate denotes the amount. In at least some example embodiments of the device, the select XY coordinates correlate with internal parts of a plurality of wells on the support surface 23, and a Z coordinate relates to the amount of the liquid mixture to be deposited in the plurality of wells on the support surface 23. In at least some example embodiments, the device comprises a control unit 1000, which is configured to select one sub-plurality of liquids from the set for each of the select XY coordinates. The sub-plurality, which is selected for each of the XY select coordinates, includes at least some liquids from the set, which are to be produced in the liquid mixture at the select XY coordinate. All of the select XY coordinates may have at least one sub-plurality of liquids from the set selected. After all sub-pluralities of liquids from the set are assigned to all of the XY coordinates, a user may provide a signal to start the device via the user interface. The control unit 1000 may be configured to generate control signals, which are used to control the device so that the various liquid mixtures are produced.

In at least some preferred example embodiments of the device, each of the liquid dispensers in the plurality 44 of liquid dispensers is configured to dispense up to a hundred thousand (100000) drops per second. In at least some preferred example embodiments of the device, each of the liquid dispensers in the plurality 44 of liquid dispensers is configured to dispense drops with a volume in a range between one (1) picoliter to a hundred (100) nanoliters. In at least some preferred example embodiments of the device, the device comprises fifty or more, or five hundred or more, or ten thousands or more liquid dispensers in the plurality 44 of liquid dispensers.

In at least some example embodiments of the device, the support surface 23 may be made of a bendable material or a non-bendable material. In at least some example embodiments of the device, the support surface 23 may be detachable or non-detachable relative to the device. In at least some example embodiments of the device, the support surface 23 is a container filled with a liquid in which the carrier drops 10 (10a, 10b . . . ) land. In at least some example embodiments of the device, the support surface 23 is a flat surface.

In at least some example embodiments of the device, the carrier drop 10 includes anti-solvent liquids (such as, but not limited to: water, tert-butanol, ethanol), at the time of dispensing, so that liquids (and/or materials and/or components located in the liquids) in drops that collide with the carrier drop 10, do not solve in the carrier drop 10 comprising the anti-solvent liquids.

In at least some example embodiments of the device, the plurality 44 of liquid dispensers are configured to dispense drops of a first liquid, and the support surface 23 is filled or covered with a second liquid, and the first liquid and the second liquid are immiscible. The first liquid and the second liquid are immiscible and do not mix with each other, and the carrier drops 10 (10a, 10b . . . ) may remain intact, may not merge amongst each other, and may be protected from evaporation immediately after landing in the second liquid, which is located in or on the support surface 23.

In at least some example embodiments of the device, at least two liquid dispensers are arranged in an array, such as a linear array, a circular array, a mesh array or any other suitable type of array comprising of the at least two liquid dispensers arranged in an ordered manner. In the example embodiment of the device as depicted in FIG. 1, the at least two liquid dispensers 2,3 are arranged in a first array 11, which is the linear array. Each of the carrier drops 10 (10a, 10b . . . ) in the stream 33 has a trajectory that passes by the at least two liquid dispensers 2,3 configured to dispense drops towards the trajectory of the carrier drop 10 in a synchronised manner, so that the carrier drop 10 sequentially collides with the drops that comprise liquids from the sub-plurality of liquid dispensers and are dispensed by at least some of the at least two liquid dispensers 2,3, which may be arranged in the first array 11.

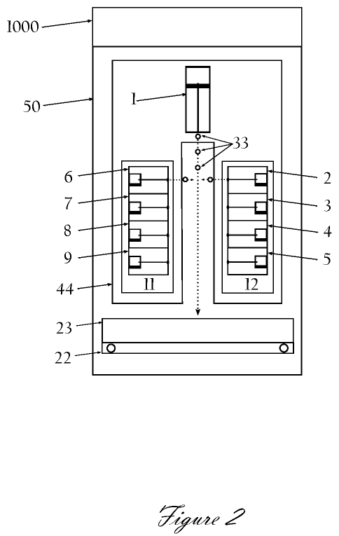

In at least some example embodiments of the device, such as depicted in FIG. 2, at least some of liquid dispensers 2-9 in the plurality 44 of liquid dispensers 1-9 are arranged in at least the first array 11 and a second array 12, wherein the first array 11 and the second array 12 are configured to dispense drops towards a single stream 33 of the carrier drops 10 (10a, 10b . . . ). In at least some example embodiments of the device, the device comprises of at least the first array 11 being configured to dispense drops towards at least a first stream 33 of carrier drops 10 (10a, 10b . . . ), and at least the second array 12 may be configured to dispense drops towards at least a second stream 33 of carrier drops 10 (10a, 10b . . . ). Two or more liquid dispensers in the plurality 44 may be configured to dispense two or more separate streams 33 of carrier drops 10 (10a, 10b . . . ).

In at least some example embodiments of the device, the device comprises a mechanisation 22 (FIG. 1) that may be configured to move the support surface 23 in at least one direction relative to the plurality 44 of liquid dispensers, so that the carrier drops 10 (10a, 10b . . . ) in the stream 33 land at the select XY coordinates on the support surface 23. In at least some example embodiments of the device, the device comprises the mechanisation 23 being configured to move the plurality 44 of liquid dispensers in at least one direction relative to the support surface 23, so that the carrier drops (10a, 10b . . . ) in the stream 33 land at the select XY coordinates coordinates on the support surface 23.

In at least some example embodiments, the device comprises a mixing chamber 50 (FIG. 2) which is a partially or a fully closed compartment of the device wherein the plurality 44 of liquid dispensers and the support surface 23 are positioned, and the mixing chamber 50 is the fully closed compartment during a time in which the drops are in-flight. In at least some example embodiments of the device, the device comprises at least one environment regulator positioned in the mixing chamber 50, and the environment regulator is configured to regulate at least a temperature and a humidity in the mixing chamber 50 over time. Preferably, the temperature and the humidity in the mixing chamber 50 is adjusted by using the at least one environment regulator so that evaporation of drops is minimised at least during the time in which the drops are in-flight. In at least some example embodiments of the device, the device may be used to produce custom solid compounds by way of evaporating the carrier drops 10(10a, 10b . . . ) after the carrier drops 10(10a, 10b . . . ) land on the support surface 23 or before the carrier drops 10(10a, 10b . . . ) land on the support surface 23. In the example embodiment wherein the carrier drops 10(10a, 10b . . . ) evaporate before landing on the support surface 23, the at least one environment regulator in the mixing chamber 50 may be configured to produce an appropriate temperature and humidity in the mixing chamber 50 so that the carrier drops 10(10a, 10b . . . ) evaporate before just before landing on the support surface 23.

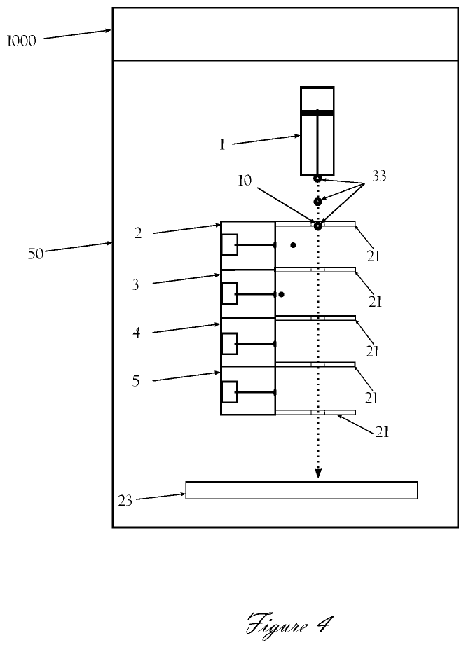

In at least some example embodiments of the device, the device comprises electric plates 21 (FIG. 4) arranged perpendicularly to a path of at least one drop dispensed from at least one liquid dispenser 1, wherein the electric plates 21 have holes through which the at least one drop passes, the at least one liquid dispenser 1 is configured to provide an electric charge to the at least one drop, and the electric plates 21 are configured so that the electric plates 21 exert electric fields on the at least one drop in a synchronised manner, so that the at least one drop is manipulated with the electric fields exerted by the electric plates 21 during the time in which the at least one drop passes through the electric fields exerted by the electric plates 21. In at least some example embodiments of the device, the electric plates 21 are mounted perpendicularly to the trajectory of the carrier drop 10.

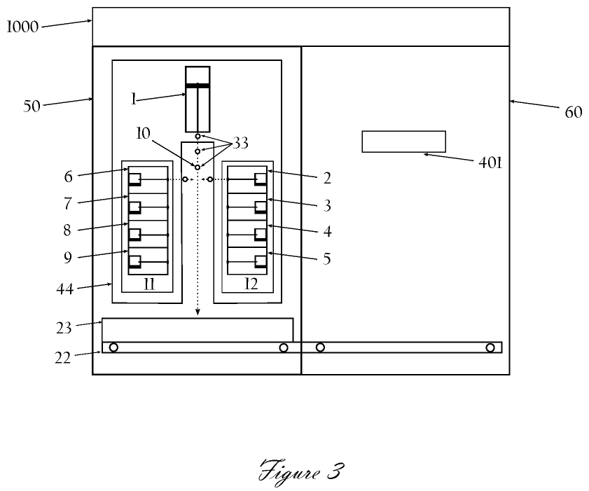

In at least some example embodiments of the device, the device comprises a processing chamber 60 (FIG. 3) wherein at least one processing element 401 is installed, and the at least one processing element 401 is configured to remove the anti-solvent liquids from all carrier drops 10 (10a, 10b . . . ) located in the processing chamber 60 and comprising the anti-solvent liquids.

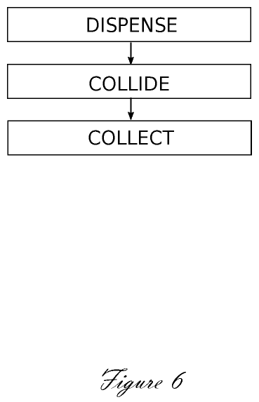

Hereinafter at least some example embodiments of the method for mixing liquids (hereinafter: the method) are described. In at least some example methods, the user provides the user data, including but not limited to defining liquid mixtures to be produces, defining settings related to the select XY coordinates, concentration variations, and other relevant user data. After the user data is provided by the user, the control unit 1000 may generate control signals, which may be used to control the device to produce the liquid mixtures (or solid compounds). In at least some example embodiments, the method comprises selecting one sub-plurality of liquids from the set for each select XY coordinate on the support surface 23 whereat the liquid mixtures shall be produced using the device and the method disclosed herein, by using the control unit 1000. One sub-plurality of liquids is selected for each of the select XY coordinates. In the next step, the user may give the signal to start the device. In at least some example embodiments of the method for mixing liquids disclosed herein, the method comprises dispensing, by using at least a plurality 44 of liquid dispensers 1-3 (FIG. 1), at least one carrier drop 10 and at least drops that comprise liquids from a select sub-plurality of liquid dispensers, wherein the carrier drop 10 sequentially collides and merges with the drops that comprise the liquids from the select sub-plurality of liquid dispensers, so that the liquids from the select sub-plurality of liquid dispensers are located in the carrier drop 10 during flight, and collecting the carrier drop 10 by using a support surface 23. Main steps of the method are schematically depicted in FIG. 6. In at least some example embodiments of the method, the method includes using any one of the devices according to the present disclosure. When all of the carrier drops 10 (10a, 10b . . . ) in the stream 33 land on the support surface 23 at all of the select XY coordinates, such a correct depositing of the carrier drops 10 (10a, 10b . . . ) in the select XY coordinates on the support surface 23 results in the liquid mixtures produced on the support surface 23. After the liquid mixtures (or solid compounds) are produced using at least some example embodiments of the devices and methods according to the present disclosure, the liquid mixtures are removed from the device. At least some example embodiments of the method include using any one of the example embodiments of the device as disclosed herein.

At least some example embodiments according to the present disclosure relate to a device and a method for preparing samples for assays. Hereinafter, at least some example embodiments of devices and methods that relate to the device and the method for preparing samples for assays are described in more detail. In at least some example embodiments, the device for preparing samples (hereinafter: the device) is used to prepare a sample for an assay, such as: high-throughput protein crystallography screening, qPCR for gene-expression studies or qPCR for SNP genotyping, ddPCR assays, a polymorph screening of engineered particles and/or compounds, any other molecular-based assays, cell encapsulation into multi-component materials studies, single-cell line viability screening, a multiplex screening of biomarkers in cells, compound libraries screening, high-throughput forensic genotyping—Short Tandem Repeat (STR), gene expression regulation assay, ADMET (Absorption, Distribution, Metabolism, Elimination, Toxicity) assay, protein-protein interaction assays using yeast two-hybrid system, protein-DNA interaction assays using yeast one-hybrid system, microorganism activity/sensitivity assessment screening, antifungal screening on a chip, other assays wherein microorganisms are screened, biosensor validation and screening assays, rapid automated immunoassay or any other assay of interest, by using the method for preparing samples for assays (hereinafter: the method) according to the present disclosure.

In at least some example embodiments, the device is used to prepare the samples for the assay from a set of substances. The set may include substances, such as: soluble proteins, chemical additives, liquid substances used for pH control of the sample, isolated mRNA of interest, isolated gDNA of interest, isolated DNA of interest, isolated RNA of interest, regulatory plasmids, at least one DNA fragment (such as: siRNA), primers, probes, RT-qPCR Master Mix, qPCR Master mix, Master Mix, a desired concentration of APIs, solvent, anti-solvent dispersion (such as: methanol, ethanol, water, dimethyl sulfoxide (DMSO), ethyl acetate, toluene), polymers (such as: hydroxypropyl methyl cellulose (HPMC), poly vinyl alcohol (PVA), Eudragit® derivates, polyvinylpyrroldone (PVP), polyethylene glycol (PEG)), pharma polymers, surfactants (such as: polysorbate 20 Tween—Tween®, polysorbate 80—Tween 80®), humectants (such as: PEG, glycerol), at least one viscosity modifier (such as: PEG, glycerol), cells (such as: cells with excreted proteases, mammalian cells, E. coli cells, yeast cells, microbial cells), protein biomarkers (such as: protein metalloproteinase (MMP), various FRET-based protease substrates with or without specific inhibitors), cell encapsulation polymer used for cell encapsulation, pathogens, growth factors, metabolites, chemical of interest, a liquid used for achieving a varying concentration of any of the components listed herein, primer beads, PCR inhibitors, plasmids (such as: linearised vector encoding for bait (such as pGBKT7) protein and pray (such pGADT7) protein), DNA fragment encoding for two potential interactors (proteins) of interest, linearised vectors, fungal solution, antifungal components, at least one type of enzymes (such as HRP, ligase, polymerase), antibodies, chemical sensors, biosensors, or any other substance of interest which may be prepared for assays using the devices and the method disclosed herein. The sample is prepared, by using the device and the method, from the set of substances. The sample includes two or more substances from the set. The device comprises a plurality 44 of liquid dispensers. In an example embodiment of the device as depicted in FIG. 1, the plurality 44 of liquid dispensers comprises liquid dispensers 1-3.

The set of substances is held in a plurality of containers in at least some of the liquid dispensers in a plurality 44 of liquid dispensers, which comprises at least the liquid dispensers 1-3, in the device. Each of the substances from the set may be suspended at a known concentration in an appropriate liquid held in a separate container in at least one liquid dispenser in the plurality 44 of liquid dispensers in the device. In at least some example embodiments of the device, the device comprises a user interface configured to provide user data, which may include method settings, device settings and any other relevant user data. The device comprises a support surface 23. In at least some example embodiments of the device, the device may include a control unit 1000, which may be configured to select a series of select XY coordinates on the support surface 23 whereat the samples will be prepared using the device and the method. The series of select XY coordinates coordinates comprises all select XY coordinates, which represent the locations whereat the samples will be prepared. Each of the select XY coordinates in the series of select XY coordinates includes an X and a Y coordinate relative to the support surface 23. In at least some example embodiments, a Z coordinate may be a part of the series of select XY coordinates, and the Z coordinate may correspond to an amount of the sample to be prepared. In one example, the X and the Y coordinates are located on the plane of the support surface 23, and the Z coordinate denotes the amount. In at least some example embodiments of the device, the select XY coordinates correlate with internal parts of a plurality of wells of a standard microtiter plate or any other plastic consumable used with the device, which may be positioned on the support surface 23, and a Z coordinate may relate to an amount of the samples to be deposited in the plurality of wells of the standard microtiter plate or any other plastic consumable used with the device and positioned on the support surface 23. In at least some example embodiments, the device comprises the control unit 1000, which is configured to assign two or more substances from the set at a ratio of interest, an amount of interest, and a concentration of interest, to each of the select XY coordinates in the series of select XY coordinates. The two or more substances, which are assigned to each of the select XY coordinates, include two or more substances from the set, which are to be prepared in the sample on the support surface 23. All select XY coordinates in the series of select XY coordinates may have at least two substances from the set assigned. After all substances from the set are assigned to at least all select XY coordinates in the series of select XY coordinates, the user may provide a signal to start the device via the user interface. The control unit 1000 may be configured to generate control signals, which are used to control the device so that the samples are prepared on the support surface 23. The samples, which are prepared using the device and the method according to the present disclosure, comprise two or more substances from the set.

In at least one preferred example embodiment of the device, such as depicted in FIG. 1, the device comprises the plurality 44 of liquid dispensers 1-3 being configured to dispense drops, so that at least a carrier drop 10 sequentially collides with N drops that comprise a first substance and M drops that comprise at least a second substance, so that the first substance and the second substance are located at a ratio of interest (N:M) and at an amount of interest (N+M) in the carrier drop 10 during flight, and the support surface 23 being configured to collect the carrier drop 10 on a select XY coordinate on the support surface 23, and in this way, the samples are prepared for the assay on the support surface 23. N is a number of drops that comprise the first substance is. M is a number of drops that comprise the second substance. The ratio of interest is noted herein as N:M, the amount of interest is noted herein as N+M. Each of the liquid dispensers in the plurality 44 comprising of at least the liquid dispensers 1-3 comprises at least a container for holding liquids to dispense, a drop generating element positioned and configured to cause at least one drop to dispense, a nozzle through which the at least one drop is ejected from, a capillary which connects the container with the nozzle so that the liquid runs from the container through the capillary and out through the nozzle for the at least one drop to be dispensed, and synchronisation means, which enable each of the liquid dispensers to operate in a synchronised manner with other liquid dispensers in the plurality 44 of liquid dispensers. In some example embodiments, a single container is configured to supply liquids to two or more nozzles in the plurality 44 of liquid dispensers. In some example embodiments, two or more containers are configured to supply liquids to a single nozzle in the plurality 44 of liquid dispensers. At least one liquid dispenser, such as the liquid dispenser 1 in FIG. 1, is configured to dispense the carrier drop 33 or a stream 33 of carrier drops 10 (10a, 10b . . . ), wherein the stream 33 comprises a first carrier drop 10, a second carrier drop 10a, and at least a third carrier drop 10b. The plurality 44 of liquid dispensers are configured so that the first carrier drop 10 collides with a first number of N drops and a first number of M drops, the second carrier drop 10a collides with a second number of N drops and a second number of M drops, the third carrier drop 10b collides with a third number of N drops and a third number the M drops, and so on, until all of the carrier drops 10 (10a, 10b . . . ) in the stream 33 collide with at least some number of N drops and some number of M drops, and all of the carrier drops 10 (10a, 10b . . . ) in the stream 33 land on the support surface 23. Collisions occur between each of the carrier drops 10 (10a, 10b . . . ) in the stream 33 and all drops that carry liquids from any one appropriate sub-plurality 44 of liquids from the set assigned to the each of the carrier drops 10 (10a, 10b . . . ). It is understood that any appropriate combinations of liquids may be included in the set, or in any sub-plurality. All of the drops are dispensed. The stream 33 may be continuous or discontinuous.

In at least some preferred example embodiments of the device, the support surface 23 is configured to collect each of the carrier drops 10 (10a, 10b . . . ) in the stream 33 at a unique select XY coordinate on the support surface 23. In at least some preferred example embodiments of the device, the support surface 23 is configured to collect two or more carrier drops 10 (10a, 10b . . . ) from the stream 33 at a same select XY coordinate on the support surface 23, depending on the amount of interest (N+M) of the first substance and at least the second substance to be prepared for the assay. In at least some preferred example embodiments of the device, at least some of liquid dispensers are configured to dispense drops that collide with the carrier drops 10 (10a, 10b . . . ) and aggregately comprise more than two substances of interest, so that the more than two substances substances are prepared at the ratio of interest, the amount of interest, and the concentration of interest, at all available select XY coordinates on the support surface 23. In at least some preferred example embodiments of the device, the device is configured to prepare the sample on all of the available select XY coordinates on the support surface 23, so that the all of the available select XY coordinates on the support surface 23 are filled with at least two substances at the ratio of interest, the amount of interest, and a concentration of interest, and/or no substances of interest.

In at least some preferred example embodiments of the device, each of the liquid dispensers in the plurality 44 of liquid dispensers is configured to dispense up to a hundred thousand (100000) drops per second. In at least some preferred example embodiments of the device, each of the liquid dispensers in the plurality 44 of liquid dispensers is configured to dispense drops with a volume in a range between 1 picoliter to 100 nanoliters. In at least some preferred example embodiments, the device comprises fifty or more, or five hundred or more, or ten thousands or more liquid dispensers in the plurality 44 of liquid dispensers.

In at least some example embodiments of the device, the support surface 23 is made of a bendable material or a non-bendable material. In at least some example embodiments of the device, the support surface 23 is detachable or non-detachable relative to the device. In at least some example embodiments of the device, the support surface 23 is a container filled with a liquid in which the carrier drops 10 (10a, 10b . . . ) land. In at least some example embodiments of the device, the support surface 23 is a flat surface. In at least some example embodiments of the device, the support surface 23 holds a standard microtiter plate or any other at least one plastic consumable, which is used with the device, so that the sample is prepared on the select XY coordinates that correspond to locations of the plurality of wells in the microtiter plate or in/on the any other plastic consumable, which is positioned on the support surface 23.

In at least some example embodiments of the device, the carrier drops 10 (10a, 10b . . . ) include anti-solvent liquids (such as, but not limited to: water, tert-butanol, ethanol), at a time of dispensing, so that two or more substances in drops that collide with the carrier drops, do not solve in the carrier drops 10 (10a, 10b . . . ) comprising the anti-solvent liquids.

In at least some example embodiments of the device, at least one liquid dispenser in the plurality 44 of liquid dispensers is configured to dispense drops that contain no substances of interest and collide with the carrier drop 10, and the plurality 44 of liquid dispensers is configured to collide the carrier drop 10 with at least some of the drops that contain no substances of interest, so that a lower concentration of the first substance and at least the second substance is achieved in the carrier drop 10, or no substances are contained in the carrier drop 10 when the carrier drop 10 lands, so that the carrier drop 10, which contains no substances, is used for control purposes in the sample, which is prepared using the device and the method according to the present disclosure.

In at least some example embodiments of the device, the plurality 44 of liquid dispensers are configured to dispense drops of a first liquid, and the support surface 23 is filled or covered with a second liquid, and the first liquid and the second liquid are immiscible. The first liquid and the second liquid are immiscible and do not mix with each other, and the carrier drops 10 (10a, 10b . . . ) remain intact, do not merge amongst each other, and are protected from evaporation immediately after landing in the second liquid, which located in or on the support surface 23.

In at least some example embodiments of the device, at least two liquid dispensers are arranged in an array, such as a linear array, a circular array, a mesh array or any other suitable type of array comprising two or more liquid dispensers arranged in an ordered manner. In the example embodiment of the device as depicted in FIG. 1, the at least two liquid dispensers 2,3 are arranged in a first array 11, which is the linear array. Each of the carrier drops 10 (10a, 10b . . . ) has a trajectory that passes by the at least two liquid dispensers 2,3 configured to dispense the N drops and the M drops towards the trajectory of the carrier drop 10 in a synchronised manner, so that the carrier drop sequentially collides with the N drops and the M drops dispensed by at least some of the at least two liquid dispensers 2,3, which may be arranged in the array 11.

In at least some example embodiments of the device, such as depicted in FIG. 2, at least some of liquid dispensers 2-9 in the plurality 44 of liquid dispensers 1-9 are arranged in at least the first array 11 and a second array 12, wherein the first array 11 and the second array 12 are configured to dispense drops towards a single stream 33 of the carrier drops 10 (10a, 10b . . . ). In at least some example embodiments of the device, at least the first array 11 is configured to dispense drops towards a first stream 33 of carrier drops 10 (10a, 10b . . . ), and at least the second array 12 is configured to dispense drops towards a second stream 33 of carrier drops. Two or more liquid dispensers in the plurality 44 may be configured to dispense two or more separate streams 33 of carrier drops 10 (10a, 10b . . . ).

In at least some example embodiments of the device, the device comprises a mechanisation 22 (FIG. 1) being configured to move the support surface 23 in at least one direction relative to the plurality 44 of liquid dispensers, and/or to move the plurality 44 of liquid dispensers in at least one direction relative to the support surface 23, so that the carrier drops 10 (10a, 10b) in the stream 33 land in the series of select XY coordinates coordinates on the support surface 23.

In at least some example embodiments, the device comprises a mixing chamber 50 (FIG. 2) which is a partially or a fully closed compartment of the device wherein the plurality 44 of liquid dispensers, and the support surface 23 are positioned, and the mixing chamber 50 is the fully closed compartment during a time in which the drops are in-flight. In at least some example embodiments, the device comprises at least one environment regulator positioned in the mixing chamber 50, and the environment regulator may be configured to regulate at least a temperature, a humidity, and CO₂ concentration in the mixing chamber 50 over time.

In at least some example embodiments of the device, the device comprises electric plates 21 (FIG. 4) arranged perpendicularly to a path of at least one drop dispensed from at least one liquid dispenser 1, wherein the electric plates 21 have holes through which the at least one drop passes, the at least one liquid dispenser 1 is configured to provide an electric charge to the at least one drop, and the electric plates 21 are configured so that the electric plates 21 exert electric fields on the at least one drop in a synchronised manner, so that the at least one drop is manipulated with the electric fields exerted by the electric plates 21 during the time in which the at least one drop passes through the electric fields exerted by the electric plates 21. In at least some example embodiments of the device and the method, the electric plates 21 are mounted perpendicularly to the trajectory of the carrier drops 10 (10a, 10b . . . ).

In at least some example embodiments of the device, the device comprises a processing chamber 60 (FIG. 3) wherein at least one processing element 401 is installed and configured to regulate at least temperature, humidity, and CO₂ concentration in the processing chamber 60, so that the sample, which was prepared on the support surface 23 and is located in the processing chamber 60, is incubated. In at least some example embodiments of the device, the mechanisation 22 is configured to move the support surface 23 to the processing chamber 60.

Hereinafter at least some example embodiments of the method for preparing samples for assays are described. In at least some example embodiments of the method, the user inputs the user data, including but not limited to the list of substances included in the set, defining select XY coordinates on a support surface 23 whereat the samples will be prepared using the device and the method, and other relevant user data such as device settings and method settings. After the user data is provided by the user, the control unit 1000 may generate the control signals, which may be used to control the device. In at least some example embodiments, the method comprises selecting, by using the control unit 1000, a series of select XY coordinates on the support surface 23, and assigning two or more substances from the set to each select XY coordinate in the series of select XY coordinates, by using the control unit 1000, so that the sample to be prepared using the device and the method is represented by the series of select XY coordinates coordinates, wherein each of the select XY coordinates has two or more substances assigned. In the next step, the user may give the signal to start the device. In at least some example embodiments, the method comprises dispensing and colliding drops by using at least a plurality 44 of liquid dispensers 1-3 (FIG. 1), so that at least one carrier drop 10 sequentially collides with a pre-determined number of N drops that comprise a first substance and M drops that comprise at least a second substance, so that the first substance and the second substance are located at a ratio of interest (N:M) and at an amount of interest (N+M) in the carrier drop 10 during flight, and configuring the support surface 23 so that the carrier drop 10 lands at one select XY coordinate on the support surface 23 and the sample is prepared for the assay on the support surface 23 this way. Main steps of the method are schematically depicted in FIG. 6. Any other example embodiment of the method for preparing samples for the assay is possible by using any one of the example embodiments of the device disclosed herein. At least one stream 33 of carrier drops 10 (10a, 10b . . . ) may be dispensed. When all of the carrier drops 10 (10a, 10b . . . ) in the stream 33 land on the support surface 23 in the series of select XY coordinates, the combined result of such a correct depositing of the carrier drops 10 (10a, 10b . . . ) in the series of select XY coordinates coordinates on the support surface 23 results in the sample being prepared for the assay on the support surface 23. After the sample is prepared using at least some example embodiments of the devices and methods hereof, the sample is removed from the device. At least some example embodiments of the method include using any one of the devices as disclosed herein.

In at least some example embodiments wherein the sample is prepared, by using the device and the method, for the high-throughput protein/compound crystallography screening, the set may include substances, such as: one or more types of soluble proteins, various chemical additives, various liquid substances for pH control of the sample, and other possible substances of interest.

In at least some example embodiments wherein the sample is prepared, by using the device and the method, for qPCR gene expression studies or SNP genotyping, the set may include substances, such as: one or more types of isolated mRNA or gDNA of interest, respectively, various primers, various probes, and may include other substances of interest. In one example, carrier drops 10 (10a, 10b . . . ) may be made of RT-qPCR Master Mix or qPCR Master mix.

In at least some example embodiments wherein the sample is prepared, by using the device and the method, for ddPCR, the set may include substances, such as: one or more types of isolated DNA/RNA of interest, one or more types of primers, one or more types of probes, and may include other substances of interest. In one example, carrier drops 10 (10a, 10b . . . ) may be made of Master mix with specific primers and probes, in another example, specific primers and probes may be located in secondary droplets, which are mixed with each of the carrier drops 10 (10a, 10b . . . ) in-flight.

In at least some example embodiments wherein the sample is prepared, by using the device and the method, for polymorph screening of medicinal particles, the set may include substances, such as: various concentration of API(s), various solvent and anti-solvent dispersions (such as methanol, ethanol, water, dimethyl sulfoxide (DMSO), ethyl acetate, toluene,) various polymers (such as hydroxypropyl methyl cellulose (HPMC), poly vinyl alcohol (PVA), Eudragit® derivates, polyvinylpyrroldone (PVP), polyethylene glycol (PEG)), various surfactants (such as such as polysorbate 20 Tween—Tween 20®, polysorbate 80—Tween 80®.), various humectants (such as PEG, glycerol), various viscosity modifiers (such as PEG, glycerol), and may include other substances of interest.

In at least some example embodiments wherein the sample is prepared, by using the device and the method, for protein biomarker assay, which may be used in the high-sensitivity and high-throughput detection and multiplex screening of protein biomarkers in cells, the set may include substances, such as: one or more types of cells (clinical samples of cells with excreted proteases), various protein biomarkers (such as but not limited to: protein metalloproteinase (MMP), various FRET-based protease substrates with or without specific inhibitors), and other possible substances of interest.

In at least some example embodiments wherein the sample is prepared for single cells encapsulation into multi-material compounds by using the device and the method hereof, the set may include substances, such as: one or more types of cells, various drugs (APIs?), various growth factors, various encapsulation polymers, various pathogens, and may include other substances of interest.

In at least some example embodiments wherein the sample prepared is a single cell line prepared for viability screening, the set may include various components such as: one or more types of cells, various APIs, various growth factors, various metabolites, various pathogens, and may include other substances of interest.

In at least some example embodiments wherein the sample is prepared, by using the device and the method, for screening compound libraries (such as activity screening of small molecules against disease-relevant target pathways, or proteins), the set may include substances, such as:one or more types of mammalian cells solution, various chemical components, various APIs, one or more type of liquid for varying concentrations of chemical or drug compounds, and may include other substances of interest.

In at least some example embodiments wherein the sample is prepared, by using the device and the method, for the high-throughput (forensic) genotyping—Short Tandem Repeat (STR) typing, the set may include include substances, such as: one or more types of cells, various primer beads, various concentrations of PCR inhibitors, and other substances of interest.

In at least some example embodiments wherein the sample is prepared, by using the device and the method, for gene expression regulation (such as gene induction, gene inhibition, gene silencing assays) the set may include include substances, such as: one or more types of E. coli cells, various regulatory plasmids, various DNA fragments (such as siRNA), and may include other substances of interest.

In at least some example embodiments wherein the sample is prepared, by using the device and the method, for ADMET screening, the set may include include substances, such as: one or more types of cells of interest, various APIs, various excipient dispersions, various pharma polymers, various surfactants, various humectants, various viscosity modifiers, and may include other substances of interest.

In at least some example embodiments wherein the sample is prepared, by using the device and the method, for protein-protein interaction screening using yeast two-hybrid system, the set may include include substances, such as: one or more types of yeast cells (pre-prepared), various plasmids such as linearised vector encoding for bait (such as but not limited to pGBKT7) protein, and pray (such as but not limited to pGADT7) protein, various DNA fragments encoding for two potential interactors (proteins) of interest, and other possible substances of interest.

In at least some example embodiments wherein the sample is prepared, by using the device and the method, for protein-DNA interaction screening using yeast one-hybrid system, the set may include include substances, such as: one or more types of yeast cells (pre-prepared), various linearised vectors, various DNA fragments, and other substances of interest.

In at least some example embodiments wherein the sample is prepared, by using the device and the method, for activity/sensitivity assessment of microorganisms, the set may include include substances, such as: one or more microbial cells, various APIs (such as but not limited to antibiotics), various fluids to achieve different concentrations, other substances of interest to affect the microorganisms, or other substances of interest.

In at least some example embodiments wherein the sample is prepared, by using the device and the method, for antifungal screening on a chip, the set may include substances, such as: one or more types of fungal solutions, various antifungal substances, and other substances of interest.

In at least some example embodiments wherein the sample is prepared, by using the device and the method, for biosensor validation and screening assays, the set may include various components such as: one or more types of biosensors to be validated, one or more types of metabolites (samples), various enzymes (e.g. RP), various antibodies, various chemical sensors, and may include other substances of interest.

At least some example embodiments according to the present disclosure may relate to a product of the device and the method for preparing samples for assays. In at least some example embodiments of the product, the product incudes a sample prepared for an assay by using any one of the devices and/or any one of the methods for preparing samples for assays hereof.

At least some example embodiments according to the present disclosure relate to a device and a method for bioprinting multi-component 3D objects. Hereinafter, at least some example embodiments of devices and methods that relate to the device and the method for bioprinting multi-component 3D objects are described in more detail. In at least some example embodiments, the device for bioprinting multi-component 3D objects (hereinafter: the device) is used to bioprint a multi-component 3D object by using the method for bioprinting multi-component 3D objects (hereinafter: the method) according to the present disclosure. The multi-component 3D object to be bioprinted using the device and the method may be any bioprintable multi-component 3D object, such as: an organ on a chip, a tissue, a scaffold, an organ, or any other bioprintable multi-component 3D object bioprinted from a plurality of components. In at least some example embodiments, the device and the method are used to bioprint the multi-component 3D object from a set of components. The set of components may include components, such as: cells, biomaterials for scaffolds, growth media, growth serum, growth factors, or other materials to aid the bioprinting process. The multi-component 3D object is bioprinted, by using the device and the method, from the set of components suspended in appropriate liquids. The device comprises a plurality 44 of liquid dispensers. In an example embodiment of the device as depicted in FIG. 1, the plurality 44 of liquid dispensers comprises liquid dispensers 1-3.

The set of components is held in a plurality of containers in at least some of the liquid dispensers in the plurality 44 comprising of at least the liquid dispensers 1-3 in the device. Each component from the set may be suspended at a known concentration in a separate liquid, which is held in a separate container in at least one liquid dispenser in the plurality 44 of liquid dispensers in the device. In at least some example embodiments of the device, the device comprises a user interface configured to provide user data, which may include a 3D model of the multi-component 3D object to be bioprinted using the device, a printing resolution, a Z layer height and other relevant user data that relate to bioprinting the multi-component 3D object. In at least some example embodiments of the device, the device comprises a control unit 1000, which is configured to convert the 3D model of the multi-component 3D object into a series of n successive XYZ coordinates, so that the series of n successive XYZ coordinates represent the 3D model. The series of n successive XYZ coordinates comprises all XYZ coordinates, which represent the 3D model of the multi-component 3D object. The device comprises a support surface 23. Each of the XYZ coordinates in the series of n successive XYZ coordinates, includes an X, a Y and a Z coordinate relative to the support surface 23. In one example, the X and the Y coordinates are located on a plane of the support surface 23, and the Z coordinate denotes the height relative to the support surface 23 (defined in layers or units of distance). In at least some example embodiments, the device comprises the control unit 1000 being configured to assign one subset of components from the set to each of the XYZ coordinates in the series of n successive XYZ coordinates. The subset, which is assigned to each of the XYZ coordinates, includes components from the set, which are to be bioprinted at the each of the XYZ coordinates in the multi-component 3D object. All XYZ coordinates in the series of XYZ coordinates have at least one subset of components from the set assigned. After all subsets of components from the set are assigned to all of the XYZ coordinates in the series of n successive XYZ coordinates, the user may provide a signal to start the device via the user interface. The control unit 1000 may be configured to generate control signals, which are used to control the device so that the multi-component 3D object is bioprinted.

In at least some example embodiments of the device, such as depicted in FIG. 1, the device comprises the plurality 44 of the liquid dispensers 1-3 configured to dispense drops, so that a stream 33 of n carrier drops 10 (10a, 10b . . . ) is dispensed and each of the n carrier drops 10 (10a, 10b . . . ) in the stream 33 sequentially collides with drops that aggregately comprise one subset of components from the set, and the support surface 23 being configured so that the n carrier drops 10 (10a, 10b . . . ) in the stream 33 land in the series of n successive XYZ coordinates that represent the multi-component 3D object, so that the multi-component 3D object is bioprinted on the support surface 23. In at least some example embodiments of the device, each of the liquid dispensers in the plurality 44 comprising of at least the liquid dispensers 1-3 comprises at least a container for holding liquids to dispense, a drop generating element positioned and configured to cause at least one drop to dispense, a nozzle through which the at least one drop is ejected from, a capillary which connects the container with the nozzle so that the liquid runs from the container through the capillary and out through the nozzle for the at least one drop to be dispensed, and synchronisation means, which enable each of the liquid dispensers to operate in a synchronised manner with other liquid dispensers in the plurality 44 of liquid dispensers. In some example embodiments of the device, a single container is configured to supply liquids to two or more nozzles in the plurality 44 of liquid dispensers. In some example embodiments of the device, two or more containers are configured to supply liquids to a single nozzle in the plurality 44 of liquid dispensers. In at least one preferred example embodiment of the device and the method, the plurality 44 of liquid dispensers comprises fifty or more liquid dispensers. At least one liquid dispenser, such as the liquid dispenser 1 (FIG. 1), is configured to dispense the n carrier drops 10 (10a, 10b . . . ) in the stream 33, wherein the stream 33 comprises a first carrier drop 10, a second carrier drop 10a, and at least a third carrier drop 10b, and the plurality 44 of liquid dispensers is configured so that the first carrier drop 10 10 collides with at least drops that aggregately comprise a first subset of components, the second carrier drop 10a collides with at least drops that aggregately comprise a second subset of components, the third carrier drop 10b collides with at least drops that aggregately comprise a third subset components, and so on, until all of the n carrier drops 10 (10a, 10b . . . ) in the stream 33 collide with at least some drops that aggregately comprise at least some appropriate components from the set, and all of the n carrier drops 10 (10a, 10b . . . ) in the stream 33 land on the support surface 23. The first subset may, for example, comprise of components such as myocites, growth factors, and biopolymer gels, the second subset may, for example, comprise of components such as vascular endothelial cells, growth factors, and/or biopolymer gels, the third subset may comprise of components such as scaffolding material, growth factors, and/or biopolymer gels, a fourth subset may comprise of other biomaterials, and so on, so that the multi-component 3D object, which may be highly complex, may be bioprinted using the device. The first subset may be dispensed in, for example, five (5) drops, by using five (5) liquid dispensers from the plurality 44, which may include, for example, thirty (30) liquid dispensers in the device, and all of the five (5) drops collide and merge with the first carrier drop 10 in the stream 33 during flight. The second subset may be dispensed in, for example, seven (7) drops, by using seven (7) liquid dispensers from the plurality 44 of (in this given example) the thirty (30) liquid dispensers in the device, and all of the seven (7) drops collide and merge with the second carrier drop 10a in the stream 33 during flight. Similarly, the third subset may be dispensed in any appropriate number of drops that collide and merge with the third carrier drop 10b, and so on. Each subset of components is dispensed in one plurality of drops, which is dispensed from one appropriate sub-plurality of liquid dispensers (in the plurality 44 of liquid dispensers in the device) and collides and merges with one carrier drop 10. Collisions occur between each of the carrier drops 10 (10a, 10b . . . ) in the stream 33 and all drops that carry components from any one appropriate subset of components from the set assigned to the each of the carrier drops 10 (10a, 10b . . . ). It is understood that any appropriate combination and number of components may be included in the set, or in any of the subsets. In at least some example embodiments, the series of n successive XYZ coordinates includes a plurality of XY layers (described one after another in a Z direction) that represent 2D layers of the multi-component 3D object, and the multi-component 3D object is bioprinted by depositing the 2D layers of carrier drops (10a, 10b . . . ) on top of each other until the multi-component 3D object is bioprinted on the support surface 23. A number of carrier drops 10 (10a, 10b . . . ) in the stream 33 is marked herein as n, and the series of n successive XYZ coordinates comprise of n number of XYZ coordinates. All of the drops are dispensed. The stream 33 may be continuous or discontinuous.

In at least some example embodiments of the device, the n carrier drops 10 (10a, 10b . . . ) include a cell-carrying bio-ink, such as a biopolymer gel, at the time of dispensing. In at least some example embodiments of the device, the n carrier drops 10 (10a, 10b . . . ) include at least one type of cells suspended in the cell-carrying bio-ink, such as the biopolymer gel.

In at least some example embodiments of the device, at least two liquid dispensers are arranged in an array, such as a linear array, a circular array, a mesh array or any other suitable type of array comprising two or more liquid dispensers arranged in an ordered manner. In at least some example embodiments of device as depicted in FIG. 1, the at least two liquid dispensers 2,3 are arranged in a first array 11, which is the linear array. Each of the n carrier drops 10 (10a, 10b . . . ) has a trajectory that passes by the at least two liquid dispensers 2,3 configured to dispense drops towards the trajectory of the carrier drop 10 in the synchronised manner, so that the carrier drop 10 sequentially collides with the drops that aggregately comprise components from the subset and are dispensed by the at least two liquid dispensers 2,3, which may be arranged in the array 11. In at least some preferred example embodiments of the devices and the methods, the device comprises the linear array including two hundred (200) liquid dispensers, and the carrier drop 10 passes by all two hundred nozzles in the linear array in close proximity.

In at least some example embodiments of the device, such as depicted in FIG. 2, at least some of liquid dispensers 2-9 in the plurality 44 of liquid dispensers 1-9 are arranged in at least the first array 11 and a second array 12, wherein the first array 11 and the second array 12 are configured to dispense drops towards a single stream 33 of the carrier drops 10 (10a, 10b . . . ). In at least some example embodiments, at least the first array 11 is configured to dispense drops towards a first stream 33 of carrier drops 10 (10a, 10b . . . ), and at least the second array 12 is configured to dispense drops towards a second stream 33 of carrier drops. Two or more liquid dispensers in the plurality 44 may be configured to dispense two or more separate streams 33 of carrier drops 10 (10a, 10b . . . ).

In at least some example embodiments of the device, the device comprises a mechanisation 22 (FIG. 1) being configured to move the support surface 23 in at least one direction relative to the plurality 44 of liquid dispensers, and/or to move at the plurality 44 of liquid dispensers in at least one direction relative to the support surface 23, so that the n carrier drops 10 (10a, 10b . . . ) in the stream 33 land in the series of n successive XYZ coordinates on the support surface 23.

In at least some example embodiments of the device, the device comprises a mixing chamber 50 (FIG. 2) wherein the plurality 44 of liquid dispensers, the support surface 23, and at least one environment regulator are positioned, and the environment regulator is configured to regulate at least: a temperature, a humidity, and CO₂ concentration in the mixing chamber 50 over time.

In at least some example embodiments of the device, the device comprises a processing chamber 60 (FIG. 3) wherein at least one processing element 401 is installed and configured to regulate the temperature, humidity, CO₂ concentration and light in the processing chamber 60, so that incubation of the multi-component 3D object, which is located in the processing chamber 60, occurs. In at least some example embodiments of the device, the device comprises the mechanisation 22 being configured to move the support surface 23, which holds the multi-component 3D object, which was bioprinted using the devices and the methods disclosed herein, to the processing chamber 60.

In at least some example embodiments of the device, the device comprises electric plates 21 (FIG. 4) arranged perpendicularly to a path of at least one drop dispensed from at least one liquid dispenser 1, wherein the electric plates 21 have holes through which the at least one drop passes, the at least one liquid dispenser 1 is configured to provide an electric charge to the at least one drop, and the electric plates 21 are configured so that the electric plates 21 exert electric fields on the at least one drop in a synchronised manner, so that the at least one drop is manipulated with the electric fields exerted by the electric plates 21 during the time in which the at least one drop passes through the electric fields exerted by the electric plates 21. In at least some example embodiments of the device and the method, the electric plates 21 are mounted perpendicularly to the trajectory of the n carrier drops 10 (10a, 10b . . . ).

At least some example embodiments according to the present disclosure may relate to a product of the device and the method for bioprinting hereof. In at least some example embodiments of the product, the product includes a bioprinted multi-component 3D object, such as organ on a chip, a tissue, a scaffold, an organ, or any other bioprintable multi-component 3D object produced by using any one of the devices and any one of the methods for bioprinting according to the present disclosure.

Hereinafter at least some example embodiments of the method for bioprinting multi-component 3D objects (hereinafter: the method) are described in more detail. In at least some example embodiments of the method, the user provides the user data, including but not limited to a 3D model of the multi-component 3D object to be bioprinted, a printing resolution, and other relevant user data, by using a user interface. After the user data is provided by the user, the control unit 1000 may generate the control signals, which may be used to control the device. In at least some example embodiments, the method comprises converting the 3D model of the multi-component 3D object, by using the control unit 1000, into the series of n successive XYZ coordinates, and assigning one subset of components from the set to each XYZ coordinate in the series of n successive XYZ coordinates, by using the control unit 1000, so that the multi-component 3D object is represented by the series of n successive XYZ coordinates, wherein each XYZ coordinate has the one subset of components assigned. In the next step, the user may give the signal to start the device. In at least some example embodiments of the method for bioprinting disclosed herein, the method comprises dispensing and colliding drops, by using at least a plurality 44 of liquid dispensers 1-3, so that a stream 33 of n carrier drops 10 (10a, 10b . . . ) is dispensed and each of the n carrier drops 10 (10a, 10b . . . ) collides and merges with at least drops that aggregately comprise one subset of components from the set during flight, and collecting the n carrier drops 10 (10a, 10b . . . ) in the series of n successive XYZ coordinates, which represent the multi-component 3D object, by using the support surface 23, so that the multi-component 3D object is bioprinted on the support surface 23. Main steps of the method are schematically depicted in FIG. 6. In at least some example embodiments of the method, the method includes using any one of the devices according to the present disclosure. When all of the n carrier drops 10 (10a, 10b . . . ) in the stream 33 land on the support surface 23 in the series of n successive XYZ coordinates, such a correct additive depositing of the n carrier drops in the series of n successive XYZ coordinates on the support surface 23, results in the multi-component 3D object bioprinted on the support surface 23. After the multi-component 3D object has been bioprinted on the support surface 23 using at least some example embodiments of the devices and methods hereof, the multi-component 3D object is removed from the device. At least some example embodiments of the method include using any one of the example embodiments of the device as disclosed herein.

At least some example embodiments according to the present disclosure relate to a device and a method for 3D printing multi-material 3D objects.

Hereinafter, at least some example embodiments of devices and methods that relate to the device and the method for 3D printing multi-material 3D objects are described in more detail. In at least some example embodiments, the device for 3D printing multi-material objects (hereinafter: the device) is configured to 3D print a multi-material 3D object by using the method for 3D printing multi-material 3D objects (hereinafter: the method) according to the present disclosure. The multi-material 3D object may be any 3D printable 3D object comprising of a plurality of materials. In at least some example embodiments, the device and the method are used to 3D print the multi-material 3D object from a set of materials, and the set of materials may include materials, such as: metal powders, particles or granules, ceramic powders, particles or granules, polymers, binder materials, or other materials to aid the 3D printing process. The materials may be suspended in appropriate liquids. The multi-material 3D object is 3D printed, by using the device and the method, from the set of materials. The device comprises a plurality 44 of liquid dispensers. In an example embodiment of the device as depicted in FIG. 1, the plurality 44 of liquid dispensers comprises liquid dispensers 1-3.

The set of materials is held in a plurality of containers in at least some of the liquid dispensers in the plurality 44 comprising at least the liquid dispensers 1-3 (FIG. 1) in the device. Each of the materials from the set may be suspended in a separate liquid, which is held in a separate container in at least one liquid dispenser in the plurality 44 of liquid dispensers in the device. In at least some example embodiments of the device, the device comprises a user interface configured to provide user data, which may include a 3D model of the multi-material 3D object to be 3D printed using the device, a printing resolution, a Z layer height and other relevant user data that relate to 3D printing of the multi-material 3D object. In at least some example embodiments of the device, the device comprises a control unit 1000, which is configured to convert the 3D model of the multi-material 3D object into a series of n successive XYZ coordinates, so that the series of n successive XYZ coordinates represent the 3D model. The series of n successive XYZ coordinates comprises all XYZ coordinates, which represent the 3D model of the multi-material 3D object. The device comprises a support surface 23. Each of the XYZ coordinates in the series of n successive XYZ coordinates, includes an X, a Y and a Z coordinate relative to the support surface 23. In one example, the X and the Y coordinates are located on a plane of the support surface 23, and the Z coordinate denotes the height relative to the support surface 23 (defined in layers or units of distance). In at least some example embodiments, the device comprises the control unit 1000 being configured to assign one subset of materials from the set to each of the XYZ coordinates in the series of n successive XYZ coordinates. The subset, which is assigned to each of the XYZ coordinate, includes materials from the set, which are to be 3D printed at the each of the XYZ coordinate in the multi-material 3D object. All XYZ coordinates in the series of XYZ coordinates have at least one subset of materials from the set assigned. After all subsets of materials from the set are assigned to all of the XYZ coordinates, the user may provide a signal to start the device via the user interface. The control unit 1000 may be configured to generate control signals, which are used to control the device so that the multi-material 3D object is 3D printed.

In an example embodiment of the device, such as depicted in FIG. 1, the device comprises the plurality 44 of the liquid dispensers 1-3 configured to dispense and collide drops, wherein a stream 33 of n carrier drops 10 (10a, 10b . . . ) is dispensed and each of the n carrier drops 10 (10a, 10b . . . ) in the stream 33 sequentially collides with drops that aggregately comprise one subset of materials from the set, and the support surface 23 being configured so that the n carrier drops 10 (10a, 10b . . . ) in the stream 33 land in the series of n successive XYZ coordinates that represent the multi-material 3D object, so that the multi-material 3D object is 3D printed on the support surface 23. In at least some example embodiments of the device, each of the liquid dispensers in the plurality 44 comprising of at least the liquid dispensers 1-3 comprises at least a container for holding liquids to dispense, a drop generating element positioned and configured to cause at least one drop to dispense, a nozzle through which the at least one drop is ejected from, a capillary which connects the container with the nozzle so that the liquid runs from the container through the capillary and out through the nozzle for the at least one drop to be dispensed, and a synchronisation means, which enable each of the liquid dispensers to operate in a synchronised manner with other liquid dispensers in the plurality 44 of liquid dispensers. In some example embodiments of the device, a single container is configured to supply liquids to two or more nozzles in the plurality 44 of liquid dispensers. In some example embodiments of the device, two or more containers are configured to supply liquids to a single nozzle in the plurality 44 of liquid dispensers. In at least one preferred example embodiment of the device, the plurality 44 of liquid dispensers comprises fifty or more liquid dispensers. At least one liquid dispenser, such as the liquid dispenser 1(FIG. 1), is configured to dispense the n carrier drops 10 (10a, 10b . . . ) in the stream 33, wherein the stream 33 comprises a first carrier drop 10, a second carrier 10a drop, and at least a third carrier drop 10b, and the plurality 44 of liquid dispensers are configured so that the first carrier drop 10 collides with at least drops that aggregately comprise a first subset of materials, the second carrier drop 10a collides with at least drops that aggregately comprise a second subset of materials, the third carrier drop 10b collides with at least drops that aggregately comprise a third subset materials from, and so on, until all of the n carrier drops 10 (10a, 10b . . . ) in the stream 33 collide with at least some drops that aggregately comprise at least some appropriate materials from the set, and all of the n carrier drops (10a, 10b . . . ) in the stream 33 land on the support surface 23. The first subset may, for example, comprise of materials such as a first type of metal powder, binder materials, and a second type of metal powders, the second subset may, for example, comprise of components such as ceramic powders, and binder materials, the third subset may comprise of materials such as the second type of metal powder, a third type of metal powder, and a highly volatile liquid; a fourth subset may comprise of other materials, so that the multi-material 3D object may be 3D printed, which may be highly complex. The first subset may be dispensed in, for example, five (5) drops, by using five (5) liquid dispensers from the plurality 44, which may include, for example, thirty (30) liquid dispensers in the device, and all of the five (5) drops collide with the first carrier drop 10 in the stream 33 during flight. The second subset may be dispensed in, for example, seven (7) drops, by using seven (7) liquid dispensers from the plurality 44 of (in this given example) the thirty (30) liquid dispensers in the device, and all of the seven (7) drops collide with the second carrier drop 10 in the stream 33 during flight. Similarly, the third subset may be dispensed in any appropriate number of drops that collide and merge with the third carrier drop 10, and so on. Each subset of components is dispensed in one plurality of drops, which is dispensed from one appropriate sub-plurality of liquid dispensers (in the plurality 44 of liquid dispensers in the device) and collides and merges with one carrier drop 10. Collisions occur between each of n the carrier drops 10 (10a, 10b . . . ) in the stream 33 and all drops that carry materials from any one appropriate subset of materials from the set assigned to the each of the n carrier drops 10 (10a, 10b . . . ). It is understood that any appropriate combinations of materials may be included in the set, or in any of the subsets. In at least some example embodiments, the series of n successive XYZ coordinates includes a plurality of XY layers (described one after another in a Z direction) that represent 2D layers of the multi-material 3D object, and the multi-material 3D object is 3D printed by depositing the 2D layers of carrier drops 10 (10a, 10b . . . ) on top of each other until the multi-component 3D object is 3D printed. A number of carrier drops 10 (10a, 10b . . . ) in the stream 33 is marked herein as n, and the series of n successive XYZ coordinates comprise of n number of XYZ coordinates.

All of the drops are dispensed. The stream 33 may be continuous or discontinuous.

In at least some example embodiments of the device, the n carrier drops 10 (10a, 10b . . . ) include the binder material, such as a polymer, a photopolymer, or a water based binder, at the time of dispensing. In at least some example embodiments of the device, the n carrier drops 10 (10a, 10b . . . ) include at least one type of powders materials suspended in the binder material.

In at least some example embodiments of the device, at least two liquid dispensers are arranged in an array, such as a linear array, a circular array, a mesh array or any other suitable type of array comprising two or more liquid dispensers arranged in an ordered manner. In at least some example embodiments of the device as depicted in FIG. 1, the at least two liquid dispensers 2,3 are arranged in a first array 11, which is the linear array. Each of the n carrier drops 10 (10a, 10b . . . ) has a trajectory that passes by the at least two liquid dispensers 2,3, configured to dispense drops towards the trajectory of at least the carrier drop 10 in a synchronised manner, so that the carrier drop 10 sequentially collides with the drops that aggregately comprise materials from the subset and are dispensed by the at least two liquid dispensers 2,3, which may be arranged in the array 11. In at least some preferred example embodiments of the device, the device comprises the linear array including two hundred (200) liquid dispensers, and the carrier drop 10 passes by all two hundred nozzles in the linear array in close proximity.

In at least some example embodiments of the device, such as depicted in FIG. 2, at least some of liquid dispensers 2-9 in the plurality 44 of liquid dispensers 1-9 are arranged in at least the first array 11 and a second array 12, wherein the first array 11 and the second array 12 are configured to dispense drops towards a single stream 33 of the n carrier drops 10 (10a, 10b . . . ). In at least some example embodiments, at least the first array 11 is configured to dispense drops towards a first stream 33 of the n carrier drops 10 (10a, 10b . . . ), and at least the second array 12 is configured to dispense drops towards a second stream of carrier drops. Two or more liquid dispensers in the plurality 44 may be configured to dispense two or more separate streams 33 of carrier drops 10 (10a, 10b . . . ).

In at least some example embodiments of the device, the device comprises a mechanisation 22 (FIG. 1) being configured to move the support surface 23 in at least one direction relative to the plurality 44 of liquid dispensers, and/or to move at least the plurality 44 of liquid dispensers in at least one direction relative to the support surface 23, so that the n carrier drops in the stream 33 land in the series of n successive XYZ coordinates on the support surface 23.

In at least some example embodiments of the device, the device comprises a mixing chamber 50 (FIG. 2) wherein the plurality 44 of liquid dispensers, the support surface 23, and at least one environment regulator are positioned, and the environment regulator is configured to regulate at least: a temperature and, a humidity in the mixing chamber 50 over time. In at least some example embodiments of the device, the plurality 44 of liquid dispensers comprises a temperature regulation system configured to regulate a temperature of the materials in each of the liquid dispensers in the plurality 44 of liquid dispensers before drops are dispensed, so that the drops are dispensed at a correct temperature.

In at least some example embodiments of the device, two or more liquid dispensers are configured to dispense drops that contain a same material from the set, and the plurality 44 of liquid dispensers is configured so that each of the n carrier drops 10 (10a, 10b . . . ) collides with a different number of drops, which comprise the same material, and a desired amount and density of the same material is contained in the each of the n carrier drops 10 (10a, 10b . . . ) The desired amount and density may vary across the multi-material 3D object, which is 3D printed, so that a functionally graded multi-material 3D object is 3D printed, wherein a physical property of the functionally graded multi-material 3D object, such as density, varies in at least one direction in the functionally graded multi-material 3D object.

In at least some example embodiments of the device, the device comprises electric plates 21 (FIG. 4) arranged perpendicularly to a path of at least one drop dispensed from at least one liquid dispenser 1, wherein the electric plates 21 have holes through which the at least one drop passes, the at least one liquid dispenser 1 is configured to provide an electric charge to the at least one drop, and the electric plates 21 are configured so that the electric plates 21 exert electric fields on the at least one drop in a synchronised manner, so that the at least one drop is manipulated with the electric fields exerted by the electric plates 21 during the time in which the at least one drop passes through the electric fields exerted by the electric plates 21. In at least some example embodiments of the device, the electric plates 21 are mounted perpendicularly to the trajectory of the n carrier drops 10 (10a, 10b . . . ).

Hereinafter at least some example embodiments of the method for 3D printing multi-material 3D objects are described. In at least some example embodiments of the method, a user of the device provides user data, including but not limited to a 3D model of the multi-material 3D object to be 3D printed, a printing resolution, and other relevant user data, by using a user interface. After the user data is provided by the user, a control unit 1000 may generate the control signals, which may be used to control the device. In at least some example embodiments, the method comprises converting the 3D model of the multi-material 3D object, by using the control unit 1000, into a series of n successive XYZ coordinates, and assigning one subset of materials from the set to each XYZ coordinate in the series of n successive XYZ coordinates, by using the control unit 1000, so that the multi-material 3D object is represented by the series of n successive XYZ coordinates, wherein each of the XYZ coordinate has one subset of materials assigned. In the next step, the user may give the signal to start the device. In at least some example embodiments of the method for 3D printing disclosed herein, the method comprises dispensing and colliding drops, by using at least the plurality 44 of liquid dispensers 1-3, so that a stream 33 of n carrier drops 10 (10a, 10b . . . ) is dispensed and each of the n carrier drops 10 (10a, 10b . . . ) collides and merges with at least drops that aggregately comprise one subset of materials from the set during flight, and collecting the n carrier drops 10 (10a, 10b . . . ) in a series of n successive XYZ coordinates, which represent the multi-material 3D object, by using the support surface 23, so that the multi-material 3D object is 3D printed on the support surface 23. Main steps of the method are schematically depicted in FIG. 6. In at least some example embodiments of the method, the method includes using any one of the devices according to the present disclosure. When all of the n carrier drops 10 (10a, 10b . . . ) in the stream 33 land on the support surface 23 in the series of n successive XYZ coordinates, such a correct additive depositing of the n carrier drops in the series of n successive XYZ coordinates on the support surface 23 results in the multi-material 3D object 3D printed on the support surface 23. After the multi-material 3D object is 3D printed on the support surface 23 using at least some example embodiments of the devices and methods hereof, the multi-material 3D object is removed from the device. At least some example embodiments of the method include using any one of the example embodiments of the device as disclosed herein.

At least some example embodiments according to the present disclosure relate to a device and a method for manufacturing multi-substance tablets.

Hereinafter at least some example embodiments of devices and methods that relate to the device and the method for manufacturing multi-substance tablets are described in more detail. In at least some example embodiments, the device for manufacturing multi-substance tablets (hereinafter: the device) is configured to manufacture a plurality of multi-substance tablets for oral delivery by using the method for manufacturing the multi-substance tablets (hereinafter: the method). The plurality of multi-substance tablets may include tablets, such as: orally disintegrating tablets or orodispensible tablets (ODT tablets), tablets for buccal delivery, high porosity ODT tablets, sustained release tablets, modified release tablets, or any other multi-substance tablets for oral delivery, which are made from a plurality of substances, by using the method. In at least some example embodiments, the device and the method are used to manufacture the plurality of multi-substance tablets from a set of active pharmaceutical ingredients (hereinafter: APIs) and/or excipients, and the set comprises APIs and/or excipients, such as: at least some APIs, polymers (such as but not limited to: hydroxypropyl methyl cellulose (HPMC), poly vinyl alcohol (PVA), Eudragit® derivates, polyvinylpyrroldone (PVP), polyethylene glycol (PEG)), surfactants (such as, but not limited to polysorbate 20—Tween 20®, polysorbate 80—Tween 80® or other), liquid ink carriers (such as: water, ethanol, propanol, or any combination of the liquid ink carriers listed herein), humectants (such as but not limited to: PEG, glycerol, or other), viscosity modifiers (such as but not limited to: glycerol, PEG, or other), solid excipient powder or granulate mixture, which may consist of various diluents, binding agents, disintegrants, lubricants, tablet coatings and films, colouring agents, sustained release agents, modified release agents, or other ingredients of the plurality of multi-substance tablets. The plurality of multi-substance tablets is produced, by using the device and the method, from the set of APIs and/or excipients. The device comprises a plurality 44 of liquid dispensers. In an example embodiment of the device as depicted in FIG. 1, the plurality 44 of liquid dispensers comprises liquid dispensers 1-3.

The set of APIs and/or excipients is held in a plurality of containers in at least some of the liquid dispensers in the plurality 44 comprising of at least the liquid dispensers 1-3 in the device. Each of the APIs and/or excipients from the set may be suspended at a known concentration in a separate liquid, which is held in a separate container in at least one liquid dispenser in the plurality 44 of liquid dispensers in the device. In at least some example embodiments of the device, the device comprises a user interface configured to provide user data, which may include a 3D model of the plurality of multi-substance tablets to be manufactured using the device, various concentrations of various APIs included in the plurality of multi-substance tablets, porosity settings, sustained release settings, modified release settings, a printing resolution, a Z layer height and other relevant user data that relate to manufacturing the plurality of multi-substance tablets. In at least some example embodiments of the device and the method, the device comprises a control unit 1000, which is configured to convert the 3D model of the plurality of multi-substance tablets into a series of n successive XYZ coordinates, so that the series of n successive XYZ coordinates represents the 3D model. The series of n successive XYZ coordinates comprises all XYZ coordinates, which represent the 3D model of the plurality of multi-substance tablets. The device comprises a support surface 23. Each of the XYZ coordinates in the series of n successive XYZ coordinates, includes an X, a Y and a Z coordinate relative to the support surface 23. In one example, the X and the Y coordinates are located on a plane of the support surface 23, and the Z coordinate denotes the height relative to the support surface 23 (defined in layers or units of distance). In at least some example embodiments, the device comprises the control unit 1000, which is configured to assign one subset of APIs and/or excipients from the set to each of the XYZ coordinates in the series of n successive XYZ coordinates. The subset, which is assigned to each of the XYZ coordinates, includes APIs and/or excipients from the set, which are to be manufactured at the each of the XYZ coordinate in the plurality of multi-substance tablets. All XYZ coordinates in the series of XYZ coordinates have at least one subset of APIs and/or excipients from the set assigned. After all subsets of APIs and/or excipients from the set are assigned to all of the XYZ coordinates, the user may provide a signal to start the device via the user interface. The control unit 1000 may be configured to generate control signals, which are used to control the device so that the plurality of multi-substance tablets is manufactured.

In at least one preferred example embodiment of the device, such as depicted in FIG. 1, the device comprises the plurality 44 of liquid dispensers 1-3 configured to dispense and collide drops, wherein a stream 33 of n carrier drops 10 (10a, 10b . . . ) is dispensed and each of the n carrier drops (10a, 10b . . . ) in the stream 33 sequentially collides with drops that aggregately comprise one subset of APIs and/or excipients from the set, and the support surface 23 being configured so that the n carrier drops 10 (10a, 10b . . . ) in the stream 33 land in the series of n successive XYZ coordinates that represent the plurality of multi-substance tablets, so that the plurality of multi-substance tablets is manufactured on the support surface 23. In at least some example embodiments of the device, each of the liquid dispensers in the plurality 44 comprising of at least the liquid dispensers 1-3 comprises at least a container for holding liquids to dispense, a drop generating element positioned and configured to cause at least one drop to dispense, a nozzle through which the at least one drop is ejected from, a capillary which connects the container with the nozzle so that the liquid runs from the container through the capillary and out through the nozzle for the at least one drop to be dispensed, and synchronisation means, which enable each of the liquid dispensers to operate in a synchronised manner with other liquid dispensers in the plurality 44 of liquid dispensers. In some example embodiments of the device, a single container is configured to supply liquids to two or more nozzles in the plurality 44 of liquid dispensers. In some example embodiments of the device, two or more containers are configured to supply liquids to a single nozzle in the plurality 44 of liquid dispensers. In at least one preferred example embodiment of the device, the plurality of liquid dispensers comprises fifty (50) or more liquid dispensers. At least one liquid dispenser, such as the liquid dispenser 1 in FIG. 1, is configured to dispense the n carrier drops 10 (10a, 10b . . . ) in the stream 33, wherein the stream 33 comprises a first carrier drop 10, a second carrier drop 10a, and at least a third carrier drop 10b, and the plurality 44 of liquid dispensers may be configured so that the first carrier drop 10 collides with at least drops that aggregately comprise a first subset of APIs and/or excipients, the second carrier drop 10 collides with at least drops that aggregately comprise a second subset of APIs and/or excipients, the third carrier drop 10 collides with at least drops that aggregately comprise a third subset APIs and/or excipients from the set and so on, until all of the n carrier drops 10 (10a, 10b . . . ) in the stream 33 collide with at least some drops that aggregately comprise at least some appropriate APIs and/or excipients from the set, and all of the n carrier drops 10 (10a, 10b . . . ) in the stream 33 land on the support surface 23. The first subset may, for example, comprise of a first subset of APIs and a first subset of excipient dispersion fluids, the second subset may, for example, comprise of substances such as a second subset of excipient dispersion, liquids containing binding agents, colouring agents, and a second subset of APIs, the third subset may comprise of a third subset of APIs from the set and some highly volatile liquids; a fourth subset may comprise other substances from the set, and so on, so that the plurality of multi-substance tablets, which may be highly complex, may be manufactured using the device. The first subset may be dispensed in, for example, five (5) drops, by using five (5) liquid dispensers from the plurality 44, which may include, for example, thirty (30) liquid dispensers in the device, and all of the five (5) drops collide and merge with the first carrier drop 10 in the stream 33 during flight. The second subset may be dispensed in, for example, seven (7) drops, by using seven (7) liquid dispensers from the plurality 44 of (in this given example) the thirty (30) liquid dispensers in the device, and all of the seven (7) drops collide and merge with the second carrier drop 10 in the stream 33 during flight. Similarly, the third subset may be dispensed in any appropriate number of drops that collide and merge with the third carrier drop 10, and so on. Each of the subsets of APIs and/or excipients is dispensed in one plurality of drops, which is dispensed from one appropriate sub-plurality of liquid dispensers (in the plurality 44 of liquid dispensers in the device) and collides and merges with one carrier drop 10. Collisions occur between each of the carrier drops 10 (10a, 10b . . . ) in the stream 33 and all drops that carry APIs and/or excipients from any one appropriate subset of APIs and/or excipients from the set assigned to the each of the carrier drops 10 (10a, 10b . . . ). It is understood that any appropriate combinations of APIs and/or excipients may be included in the set, or in any of the subsets. In at least some example embodiments of the device, the series of n successive XYZ coordinates includes a plurality of XY layers (described one after another in a Z direction) that represent 2D layers of the plurality of multi-substance tablets, and the plurality of multi-substance tablets is manufactured by depositing the 2D layers of carrier drops 10 (10a, 10b . . . ) on top of each other until the plurality of multi-substance tablets is manufactured on the support surface 23. A number of carrier drops 10 (10a, 10b . . . ) in the stream 33 is marked herein as n, and the series of n successive XYZ coordinates comprise of n number of XYZ coordinates. All of the drops are dispensed. The stream 33 may be continuous or discontinuous.

In at least some example embodiments of the device, the n carrier drops 10 (10a, 10b . . . ) include a liquid ink carrier capable of carrying ingredients of the plurality of multi-substance tablets to be manufactured, at the time of dispensing. In one example, the liquid ink carrier may be water. In another example, the liquid ink carrier may be ethanol. In another example, the liquid ink carrier may be propanol. In another example, the liquid ink carrier may be other liquid or liquid mixture, which is capable of carrying ingredients of the plurality of multi-substance tablets to be manufactured using the device and the method. In at least some example embodiments of the device, the n carrier drops 10 (10a, 10b . . . ) include at least the liquid ink carrier and an excipient dispersion, so that rheological properties of the liquid ink carrier may be customised.

In at least some example embodiments of the device, at least two liquid dispensers are arranged in an array, such as a linear array, a circular array, a mesh array or any other suitable type of array comprising two or more liquid dispensers arranger in an ordered manner. In at least some example embodiments of the device as depicted in FIG. 1, the at least two liquid dispensers 2,3 are arranged in a first array 11, which is the linear array. Each of the n carrier drops 10 (10a, 10b . . . ) has a trajectory that passes by the at least two liquid dispensers 2,3 configured to dispense drops towards the trajectory of at least the carrier drop 10 in a synchronised manner, so that the carrier drop 10 sequentially collides with the drops that aggregately comprise APIs and/or excipients from the subset and are dispensed by the at least two liquid dispensers 2,3, which may be arranged in the array 11. In at least one preferred example embodiment of the device, the device comprises the linear array including two hundred (200) liquid dispensers, and the carrier drops 10 (10a, 10b . . . ) pass by all two hundred nozzles in the linear array in close proximity.

In at least some example embodiments of the device, such as depicted in FIG. 2, at least some of liquid dispensers 2-9 in the plurality 44 of liquid dispensers 1-9 are arranged in at least the first array 11 and a second array 12, wherein the first array 11 and the second array 12 are configured to dispense drops towards a single stream 33 of the carrier drops 10 (10a, 10b . . . ). In at least some example embodiments of the device, at least the first array 11 is configured to dispense drops towards a first stream 33 of carrier drops 10 (10a, 10b . . . ), and at least the second array 12 is configured to dispense drops towards at least a second stream of carrier drops. Two or more liquid dispensers in the plurality 44 may be configured to dispense two or more separate streams 33 of carrier drops 10 (10a, 10b . . . ).

In at least some example embodiments of the device, the plurality 44 of liquid dispensers is configured so that some of the n carrier drops 10 (10a, 10b . . . ) in the stream 33 collide with drops that comprise the binder agents, and some other of the n carrier drops 10 (10a, 10b . . . ) in the stream 33 collide with drops that comprise solid excipients powders or granule mixtures suspended in highly volatile liquids, which evaporate before the carrier drops 10 (10a, 10b . . . ) land, so that a high porosity of the plurality of multi-substance tablets may be achieved, and the high porosity may result in high disintegration.

In at least some example embodiments of the device, the device comprises at least some liquid dispensers, which dispense drops that collide with any of the n carrier drops, and are being configured to dispense drops that contain agents, which cause a sustained release of the APIs or a modified release of the APIs in the plurality of multi-substance tablets, so that a sustained release of the plurality of multi-substance tablets or a modified release of the plurality multi-substance tablets may be achieved.

In at least some example embodiments of the device, the device comprises a mechanisation 22 (FIG. 1) being configured to move the support surface 23 in at least one direction relative to the plurality 44 of liquid dispensers, and/or to move the plurality 44 of liquid dispensers in at least one direction relative to the support surface 23, so that the n carrier drops 10 (10a, 10b . . . ) in the stream 33 land in the series of n successive XYZ coordinates on the support surface 23.

In at least some example embodiments of the device, the device comprises a mixing chamber 50 (FIG. 2) wherein the plurality 44 of liquid dispensers, the support surface 23, and at least one environment regulator are positioned, and the environment regulator is configured to regulate at least a temperature and a humidity in the mixing chamber 50 over time.

In at least some example embodiments of the device, the device comprises electric plates 21 (FIG. 4) arranged perpendicularly to a path of at least one drop dispensed from at least one liquid dispenser 1, wherein the electric plates 21 have holes through which the at least one drop passes, the at least one liquid dispenser 1 is configured to provide an electric charge to the at least one drop, and the electric plates 21 are configured so that the electric plates 21 exert electric fields on the at least one drop in a synchronised manner, so that the at least one drop is manipulated with the electric fields exerted by the electric plates 21 during the time in which the at least one drop passes through the electric fields 21 exerted by the electric plates 21. In at least some example embodiments of the device, the electric plates 21 are mounted perpendicularly to the trajectory of the n carrier drops 10 (10a, 10b . . . ).

Hereinafter at least some example embodiments of the method for manufacturing multi-substance tablets are described. In at least some example embodiments of the method, the user inputs the user data, including but not limited to a 3D model of the plurality of multi-substance tablets to be manufactured using the device and the method, a printing resolution, and other relevant user data. After the user data is provided by the user, a control unit 1000 may generate the control signals, which may be used to control the device. In at least some example embodiments of the method, the method comprises converting the 3D model of the plurality of multi-substance tablets, by using the control unit 1000, into a series of n successive XYZ coordinates, and assigning one subset of APIs and/or excipients from the set to each of the XYZ coordinates in the series of n successive XYZ coordinates, by using the control unit 1000, so that the plurality of multi-substance tablets is represented by the series of n successive XYZ coordinates, wherein each XYZ coordinate has one subset of APIs and/or excipients assigned. In the next step, the user may give the signal to start the device. In at least some example embodiments of the method disclosed herein, the method comprises dispensing and colliding drops, by using at least a plurality 44 of liquid dispensers 1-3, so that a stream 33 of n carrier drops 10 (10a, 10b . . . ) is dispensed and each of the n carrier drops 10 (10a, 10b . . . ) collides and merges with at least drops that aggregately comprise one subset of APIs and/or excipients from the set during flight, and collecting the n carrier drops 10 (10a, 10b . . . ) in the series of n successive XYZ coordinates, which represent the plurality of multi-substance tablets, by using the support surface 23, so that the plurality of multi-substance tablets is manufactured on the support surface 23. Main steps of the method are schematically depicted in FIG. 6. When all of the n carrier drops 10 (10a, 10b . . . ) in the stream 33 land on the support surface 23 in the series of n successive XYZ coordinates, such a correct additive depositing of the n carrier drops in the series of n successive XYZ coordinates on the support surface 23 results in the plurality of multi-substance tablets manufactured on the support surface 23. In at least some example embodiments of the method, the method includes using any one of the devices according to the present disclosure. After the plurality of multi-substance tablets is manufactured on the support surface 23 using at least some example embodiments of the devices and methods hereof, the plurality of multi-substance tablets is removed from the device. At least some example embodiments of the method include using any one of the example embodiments of the device as disclosed herein.

At least some example embodiments according to the present disclosure relate to a device and a method for manufacturing micro and/or nano sized therapeutical particles.

Hereinafter at least some example embodiments of devices and methods that relate to the device and the method for manufacturing the micro and/or nano sized therapeutical particles are described in more detail. In at least some example embodiments, the device for manufacturing the micro and/or nano sized therapeutical particles (hereinafter: the device) is used to manufacture a plurality of micro and/or nano sized therapeutical particles for parerental, respiratory, or nasal delivery, wherein the therapeutical particles are in the micro and nano scale size domain, by using the method for manufacturing micro and/or nano sized therapeutical particles (hereinafter: the method) of the present disclosure. In at least some example embodiments, the device and the method are used to manufacture the plurality of micro and/or nano sized therapeutical particles from a set of APIs and/or excipients. The set comprises APIs and/or excipients, such as: at least one type of APIs (therapeutic peptides or biologically-active small molecules), biodegradable carrier polymer for sustained-release (such as: poly lactide (PLA), poly (lactic-co-glycolic) acid (PLGA)), liquid ink carriers (such as: water, dimethyl sulfoxide (DMSO), ethyl acetate), anti-solvent liquids (such as: water, tert-butanol, ethanol) or other substances, which may constitute the plurality of micro and/or nano sized therapeutical particles. The plurality of micro and/or nano sized therapeutical particles are manufactured, by using the device and the method, from the set of APIs and/or excipients. The device comprises a plurality 44 of liquid dispensers. In an example embodiment of the device as depicted in FIG. 1, the plurality 44 of liquid dispensers comprises liquid dispensers 1-3 The set of APIs and/or excipients is held in a plurality of containers in at least some of the liquid dispensers in a plurality 44 comprising of at least liquid dispensers 1-3, in the device. Each of the APIs and/or excipients from the set is suspended at a known concentration in a separate liquid, which is held in a separate container in at least one liquid dispenser in the plurality 44 of liquid dispensers in the device. In at least some example embodiments of the device, the device comprises a user interface configured to provide user data, which may include a type (or types) of therapeutic particles to be produced, sizes of therapeutic particles to be produced, various APIs, concentration variations, positions of vials and/or cartridges (for inhalation devices) that may be used with the device, and other. In at least some example embodiments of the device, the device comprises a control unit 1000, which is configured to create a series of select XY coordinates, so that the series of select XY coordinates correspond to internal parts of the vials and/or cartridges that may be used with the device. The series of select XY coordinates coordinates comprises all XY coordinates whereat the plurality of micro and/or nano sized therapeutical particles will be manufactured. The device comprises a support surface 23. Each of the XY coordinates in the series of select XY coordinates coordinates includes an X and a Y coordinate relative to the support surface 23. In at least some example embodiments, a Z coordinate may be a part of the series, and the Z coordinate corresponds to an amount of micro and/or nano sized therapeutic particles to be manufactured. In one example, the X and the Y coordinates are located on a plane of the support surface 23, and the Z coordinate denotes the amount. In at least some example embodiments of the device and the method, the select XY coordinates correlate with internal part of the vials and/or cartridges (for inhalation devices), which are positioned on the support surface 23, and a Z coordinate relates to an amount of micro and/or nano sized therapeutical particles to be deposited in the vials or cartridges (for inhalation devices), which are positioned at at least the select XY coordinates on the support surface 23. In at least some example embodiments, the device comprises the control unit 1000, which is configured to assign one subset of APIs and/or excipients from the set to each of the select XY coordinates in the series of select XY coordinates. The subset, which is assigned to each of the select XY coordinates, includes APIs and/or excipients from the set, which are to be manufactured at the each of the select XY coordinates in the plurality of micro and/or nano sized therapeutical particles produced using the device. All select XY coordinates in the series of select XY coordinates have at least one subset of APIs and/or excipients from the set assigned. After all subsets of APIs and/or excipients from the set are assigned to all of the select XY coordinates, the user may provide a signal to start the device via the user interface. The control unit 1000 may be configured to generate control signals, which are used to control the device so that the plurality of micro and/or nano sized therapeutical particles is manufactured.

In at least one preferred example embodiment of the device, such as depicted in FIG. 1, the device comprises the plurality 44 of liquid dispensers 1-3 configured to dispense and collide drops, wherein a stream 33 of n carrier drops 10 (10a, 10b . . . ) is dispensed and each of the n carrier drops 10 (10a, 10b . . . ) in the stream 33 sequentially collides with at least drops that aggregately comprise one subset of APIs and/or excipients from the set, and the support surface 23 being configured so that the n carrier drops 10 (10a, 10b . . . ) in the stream 33 land in the series of select XY coordinates, so that the plurality of micro and/or nano sized therapeutical particles is manufactured on the support surface 23 in the series of select XY coordinates. In some example embodiments of the device, each of the liquid dispensers in the plurality 44 comprising of at least liquid dispensers 1-3 comprises at least a container for holding liquids to dispense, a drop generating element positioned and configured to cause at least one drop to dispense, a nozzle through which the at least one drop is ejected from, a capillary which connects the container with the nozzle so that the liquid runs from the container through the capillary and out through the nozzle for the at least one drop to be dispensed, and synchronisation means, which enable each of the liquid dispensers to operate in a synchronised manner with other liquid dispensers in the plurality 44 of liquid dispensers. In some example embodiments of the device, a single container is configured to supply liquids to two or more nozzles in the plurality 44 of liquid dispensers. In some example embodiments of the device, two or more containers are configured to supply liquids to a single nozzle in the plurality 44 of liquid dispensers. At least one liquid dispenser, such as the liquid dispenser 1 in FIG. 1, is configured to dispense the n carrier drops 10 (10a, 10b . . . ) in the stream 33, wherein the stream 33 comprises a first carrier drop 10, a second carrier drop 10a, and at least a third carrier drop 10b, and the plurality 44 of liquid dispensers may be configured so that the first carrier drop 10 10 collides with at least drops that aggregately comprise a first subset of APIs and/or excipients, the second carrier drop 10a collides with at least drops that aggregately comprise a second subset of APIs and/or excipients, the third carrier drop 10b collides with at least drops that aggregately comprise a third subset APIs and/or excipients, and so on, until all of the n carrier drops 10 (10a, 10b . . . ) in the stream 33 collide with at least some drops that aggregately comprise at least some appropriate APIs and/or excipients from the set, and all of the n carrier drops 10 (10a, 10b . . . ) in the stream 33 land on the support surface 23. The first subset may be dispensed in, for example, five (5) drops, by using five (5) liquid dispensers from the plurality 44 of, for example, thirty (30) liquid dispensers in the device, and all of the five (5) drops collide and merge with the first carrier drop 10 in the stream 33 during flight. The second subset may be dispensed in, for example, seven (7) drops, by using seven (7) liquid dispensers from the plurality 44 of (in this given example) the thirty (30) liquid dispensers in the device, and all of the seven (7) drops collide and merge with the second carrier drop 10a in the stream 33 during flight. Similarly, the third subset may be dispensed in any appropriate number of drops that collide and merge with the third carrier drop 10b, and so on. Each subset of APIs and/or excipients is dispensed in one plurality of drops, which is dispensed from one appropriate sub-plurality of liquid dispenser(from the plurality 44 of liquid dispensers in the device) and collides and merges with one carrier drop 10. Collisions occur between each of the n carrier drops 10 (10a, 10b . . . ) in the stream 33 and all drops that carry APIs and/or excipients from any one appropriate subset of APIs and/or excipients from the set assigned to the each of the n carrier drops 10 (10a, 10b . . . ). It is understood that any appropriate combinations of APIs and/or excipients may be included in the set, or in any of the subsets. A number of carrier drops 10 (10a, 10b . . . ) in the stream 33 is marked herein as n, and the plurality of micro and/or nano therapeutic particles may include a n number of micro and/or nano sized therapeutical particles produced using the device and the method disclosed herein. All of the drops are dispensed. The stream 33 may be continuous or discontinuous.

In at least some example embodiments of the device, the n carrier drops 10 (10a, 10b . . . ) include an excipient dispersion capable of carrying ingredients of the plurality of micro and/or nano therapeutic particles to be manufactured, at the time of dispensing. In at least some example embodiments of the device, the n carrier drops 10 (10a, 10b . . . ) include at least one API and the excipient dispersion capable of carrying ingredients of the plurality of micro and/or nano therapeutic particles to be manufactured, at the time of dispensing. In at least some example embodiments of the device, the n carrier drops 10 (10a, 10b . . . ) include anti-solvent liquids (such as, but not limited to: water, tert-butanol, ethanol), at the time of dispensing, so that substances included in drops that collide with the carrier drops 10 (10a, 10b . . . ), do not solve in the carrier drops (10a, 10b . . . ) comprising the anti-solvent liquids.

In at least some example embodiments of the device, at least two liquid dispensers are arranged in an array, such as a linear array, a circular array, a mesh array or any other suitable type of array. In device as depicted in FIG. 1, the at least two liquid dispensers 2,3 are arranged in a first array 11, which is the linear array. Each of the n carrier drops 10 (10a, 10b . . . ) has a trajectory that passes by the at least two liquid dispensers 2,3 configured to dispense drops towards the trajectory of at least the carrier drop 10 in a synchronised manner, so that the carrier drop 10 sequentially collides with the drops that aggregately comprise APIs and/or excipients from the subset and are dispensed by the at least two liquid dispensers 2,3, which may be arranged in the array 11.

In at least some example embodiments of the device, such as depicted in FIG. 2, at least some of liquid dispensers 2-9 in the plurality 44 of liquid dispensers 1-9 are arranged in at least the first array 11 and a second array 12, wherein the first array 11 and the second array 12 are configured to dispense drops towards a single stream 33 of the carrier drops 10 (10a, 10b . . . ). In at least some example embodiments, at least the first array 11 is configured to dispense drops towards a first stream 33 of carrier drops 10 (10a, 10b . . . ), and at least the second array 12 is configured to dispense drops towards a second stream of carrier drops. Two or more liquid dispensers in the plurality 44 may be configured to dispense two or more separate streams 33 of carrier drops 10 (10a, 10b . . . ).

In at least some example embodiments of the device and the method, the device comprises a freeze drying element mounted on the support surface 23. The freeze drying element is configured to freeze dry the carrier drops 10 (10a, 10b . . . ) immediately after the carrier drops 10 (10a, 10b . . . ) land on the support surface 23, so that spherically shaped micro and/or nano sized therapeutical particles are produced. The freeze drying element may include substances such as liquid nitrogen.

In at least some example embodiments of the device, the device comprises a mechanisation 22 (FIG. 1) being configured to move the support surface 23 in at least one direction relative to the plurality 44 of liquid dispensers, and/or to move the plurality 44 of liquid dispensers in at least one direction relative to the support surface 23, so that the n carrier drops 10 (10a, 10b . . . ) in the stream 33 land in the series of select XY coordinates on the support surface 23.

In at least some example embodiments of the device, the device comprises a mixing chamber 50 (FIG. 2) wherein at least the plurality 44 of liquid dispensers, the support surface 23, and at least one environment regulator are positioned, and the environment regulator is configured to regulate at least a temperature and a humidity in the mixing chamber 50 over time. In at least some example embodiments, the environment regulator is configured to provide the temperature and the humidity in the mixing chamber 50 so that the carrier drops 10 (10a, 10b . . . ) evaporate or spray dry before landing in at least the select XY coordinates, so that the spherically shaped nano and/or micro sized therapeutical particles may be manufactured.

In at least some example embodiments of the device, the device comprises electric plates 21 (FIG. 4) arranged perpendicularly to a path of at least one drop dispensed from at least one liquid dispenser 1, wherein the electric plates 21 have holes through which the at least one drop passes, the at least one liquid dispenser 1 is configured to provide an electric charge to the at least one drop, and the electric plates 21 are configured so that the electric plates 21 exert electric fields on the at least one drop in a synchronised manner, so that the at least one drop is manipulated with the electric fields exerted by the electric plates 21 during the time in which the at least one drop passes through the electric fields exerted by the electric plates 21. In at least some example embodiments of the device, the electric plates 21 are mounted perpendicularly to the trajectory of the n carrier drops 10 (10a, 10b . . . ).

In at least some example embodiments of the device, the device comprises a processing chamber 60 (FIG. 3) wherein at least one processing element 401 is installed, and the at least one processing element 401 is configured to remove the anti-solvent liquids from all carrier drops 10 (10a, 10b . . . ), which are located in the processing chamber 60 and comprise the anti-solvent liquids.

Hereinafter at least some example embodiments of the method for manufacturing micro and/or nano sized therapeutic particles are described. In at least some example methods, a user inputs user data, including but not limited to the model of the plurality of micro and/or nano sized therapeutical particles to be manufactured, and other relevant user data by using a user interface. After the user data is provided by the user, a control unit 1000 may generate the control signals, which may be used to control the device. In at least some example embodiments, the method comprises crating a series of select XY coordinates coordinates, and assigning one subset of APIs and/or excipients from the set to each select XY coordinate in the series of select XY coordinates coordinates, by using the control unit 1000, so that the plurality of micro and/or nano sized therapeutical particles is represented by the series of select XY coordinates coordinates, wherein each select XY coordinate has one subset of APIs and/or excipients assigned. In the next step, the user may give the signal to start the device. In at least some example embodiments of the method for manufacturing disclosed herein, the method comprises dispensing and colliding drops, by using the plurality 44 of at least liquid dispensers 1-3, so that a stream 33 of n carrier drops 10 (10a, 10b . . . ) is dispensed and each of the n carrier drops 10 (10a, 10b . . . ) collides and merges with at least drops that aggregately comprise one subset of APIs and/or excipients from the set during flight, and collecting the n carrier drops 10 (10a, 10b . . . ) in a series of select XY coordinates, by using the support surface 23, so that the plurality of micro and/or nano sized therapeutical particles is manufactured in the series of select XY coordinates on the support surface 23. Main steps of the method are schematically depicted in FIG. 6. When all of the n carrier drops 10 (10a, 10b . . . ) in the stream 33 land on the support surface 23 in the series of select XY coordinates coordinates, such a correct additive depositing of the n carrier drops in the series of select XY coordinates on the support surface 23 results in the plurality of micro and/or nano sized therapeutical particles manufactured on the support surface 23 or in some examples, in vials or cartridges used for inhalation devices, which may be positioned on the support surface 23. In at least some example embodiments of the method, the method includes using any one of the devices according to the present disclosure. After the plurality of micro and/or nano sized therapeutical particles is manufactured using at least some example embodiments of the devices and methods hereof, the plurality of micro and/or nano sized therapeutical particles is removed from the device. At least some example embodiments of the method include using any one of the example embodiments of the device as disclosed herein.

At least some example embodiments according to the present disclosure relate to a device and a method for applying active pharmaceutical ingredients (hereinafter: APIs) and/or excipients on pre-manufactured placebos. Hereinafter, at least some example embodiments of devices and the methods that relate to the device and the method for applying APIs and/or excipients on pre-manufactured placebos are described in more detail. In at least some example embodiments, the device for applying APIs and/or excipients on pre-manufactured placebos (hereinafter: the device) is configured to apply APIs and/or excipients on a plurality of pre-manufactured placebos by using the method for applying APIs and/or excipients on pre-manufactured placebos (hereinafter: the method) according to the present disclosure. The plurality of pre-manufactured placebos may comprise of any appropriate number of pre-manufactured placebos onto which the APIs and/or the excipients may be applied, and may include pre-manufactured placebos, such as: oral tablets, capsules, orodispensible films, films in capsules, mucoadhesive films or other pre-manufactured placebos onto which the APIs and/or the excipients may be applied. In at least some example embodiments, the device and the method are used to apply a set of APIs and/or the excipients on the plurality of pre-manufactured placebos. The set comprises APIs and/or the excipients, such as: at least one type of APIs, polymers (such as but not limited to: hydroxypropyl methyl cellulose (HPMC), poly vinyl alcohol (PVA), Eudragit® derivates, polyvinylpyrroldone (PVP), polyethylene glycol (PEG)), surfactants (such as, but not limited to Polysorbate 20—Tween 20® or other), liquid ink carriers (such as but not limited to: water, ethanol, propanol, or any combination of liquid ink carriers), humectants (such as but not limited to: PEG, glycerol, or other), viscosity modifiers (such as but not limited to: glycerol, PEG, or other), sustained release agents, modified release agents, or other the APIs and/or the excipients, which may be applied to the plurality of pre-manufactured placebos. The set of the APIs and/or the excipients is applied on the plurality of pre-manufactured placebos by using the device and the method. The device comprises a plurality 44 of liquid dispensers. In an example embodiment of the device as depicted in FIG. 1, the plurality 44 of liquid dispensers comprises liquid dispensers 1-3.

The set of the APIs and/or the excipients is held in a plurality of containers in at least some of the liquid dispensers in the plurality 44 comprising of at least liquid dispensers 1-3 in the device. Each of the APIs and/or excipients from the set may be suspended at a known concentration in a separate liquid, which is held in a separate container in at least one liquid dispenser in the plurality 44 of liquid dispensers in the device. In at least some example embodiments of the device, the device comprises a user interface configured to provide user data, which may include a 3D model of the plurality of pre-manufactured placebos to be used with the device, a type (or types) of placebo therapeutics used (such as, but not limited to: oral tablets, capsules, orodispensible films, films in capsules, mucoadhesive films), defining a variable dosing setting which may be achieved across the plurality of pre-manufactured placebos; combinations of various different APIs applied across the plurality of pre-manufactured placebos, and other relevant user data that relate to applying the APIs and/or the excipients on the plurality of pre-manufactured placebos. The device comprises a support surface 23. The plurality of pre-manufactured placebos onto which the APIs and/or the excipients are applied, by using the devices and the methods disclosed herein, is positioned on the support surface 23. In at least some example embodiments of the device and the method, the device comprises a control unit 1000, which is configured to select at least some select XY coordinates on the plurality of pre-manufactured placebos, so that the select XY coordinates correspond to locations of the plurality of pre-manufactured placebos whereat the APIs and/or the excipients will be applied by using the device and the method disclosed herein. In at least some example embodiments of the device, the device comprises the control unit 1000 being configured to select one subset of the APIs and/or the excipients from the set for each of the select XY coordinates. The subset of APIs and/or excipient, which is selected for the each of the select XY coordinates, includes the subset of the APIs and/or the excipients from the set, which are to be applied at the each of the select XY coordinates on the plurality of pre-manufactured placebos. All of the select XY coordinates have at least one subset of the APIs and/or the excipients from the set selected. After all subsets of the APIs and/or the excipients from the set are selected for all of the select XY coordinates, the user may provide a signal to start the device via the user interface. The control unit 1000 may be configured to generate control signals, which are used to control the device so that the APIs and/or the excipients the set are applied on the plurality of pre-manufactured placebos located on the support surface 23.

In at least one example embodiment of the device, such as depicted in FIG. 1, the device comprises the plurality 44 of liquid dispensers 1-3 being configured to dispense drops, wherein a stream 33 of carrier drops 10 (10a, 10b . . . ) is dispensed, and each of the carrier drops 10 (10a, 10b . . . ) in the stream 33 sequentially collides with a pre-determined number of the drops that aggregately comprise one subset of the APIs and/or the excipients selected from the set, so that a pre-determined amount and combination of the APIs and/or the excipients is located in each of the carrier drops 10 (10a, 10b . . . ) in the stream 33 during flight; and the support surface 23 being configured so that the each of the carrier drops 10 (10a, 10b . . . ) in the stream 33 lands on one of the select XY coordinates on the plurality of pre-manufactured placebos positioned on the support surface 23. In at least some example embodiments of the device, each of the liquid dispensers in the plurality 44 comprising of at least liquid dispensers 1-3 comprises at least a container for holding liquids to dispense, a drop generating element positioned and configured to cause at least one drop to dispense, a nozzle through which the at least one drop is ejected from, a capillary which connects the container with the nozzle so that the liquid runs from the container through the capillary and out through the nozzle for the at least one drop to be dispensed, and synchronisation means, which enable each of the liquid dispensers to operate in a synchronised manner with other liquid dispensers in the plurality 44 of liquid dispensers. In some example embodiments of the device, a single container is configured to supply liquids to two or more nozzles in the plurality 44 of liquid dispensers. In some example embodiments of the device, two or more containers are configured to supply liquids to a single nozzle in the plurality 44 of liquid dispensers. At least one liquid dispenser, such as the liquid dispenser 1 in FIG. 1, is configured to dispense the carrier drops 10 (10a, 10b . . . ) in the stream 33, wherein the stream 33 comprises a first carrier drop, a second carrier drop 10a, and at least a third carrier drop 10b, and the plurality 44 of liquid dispensers is configured so that the first carrier drop collides with at least drops that aggregately comprise a first subset of APIs and/or excipients, the second carrier drop 10a collides with at least drops that aggregately comprise a second subset of APIs and/or excipients, the third carrier drop 10b collides with at least drops that aggregately comprise a third subset of APIs and/or the excipients from the set, and so on, until all of the carrier drops 10 (10a, 10b . . . ) in the stream 33 collide with at least some drops that aggregately comprise at least some appropriate APIs and/or excipients from the set, and all of the carrier drops 10 (10a, 10b . . . ) in the stream 33 land on the plurality of pre-manufactured placebos located on the support surface 23. The first subset may, for example, comprise of the APIs and/or the excipients such as a first mixture of APIs and viscosity modifiers, the second subset may, for example, comprise of the APIs and/or the excipients such as a second mixture of APIs and sustained release agents, the third subset may comprise of the APIs and/or the excipients such as a third mixture of APIs and humectants; a fourth subset may comprise of some other APIs and/or excipients such as colouring agents from the set, so that the APIs and/or the excipients may be applied to the plurality of pre-manufactured placebos in a highly complex manner. The first subset may be dispensed in, for example, five (5) drops, by using five (5) liquid dispensers from the plurality 44, which may include, for example, thirty (30) liquid dispensers in the device, and all of the five (5) drops collide and merge with the first carrier drop 10 in the stream 33 during flight. The second subset may be dispensed in, for example, seven (7) drops, by using seven (7) liquid dispensers from the plurality 44 of (in this given example) the thirty (30) liquid dispensers in the device, and all of the seven (7) drops collide and merge with the second carrier drop 10a 10 in the stream 33 during flight. Similarly, the third subset may be dispensed in any appropriate number of drops that collide and merge with the third carrier drop 10b 10, and so on. Each subset of APIs and/or excipients is dispensed in one plurality of drops, which is dispensed from one appropriate sub-plurality of liquid dispensers (in the plurality 44 of liquid dispensers in the device) and collides and merges with one carrier drop 10. Collisions occur between each of the carrier drops 10 (10a, 10b . . . ) in the stream 33 and all drops that carry the APIs and/or the excipients from any one appropriate subset of the APIs and/or the excipients from the set assigned to the each of the carrier drops 10 (10a, 10b . . . ). It is understood that any appropriate combinations of the APIs and/or the excipients may be included in the set, or in any of the subsets. In at least some example embodiments of the device, the series of select XYZ coordinates includes multiple layers on the surface of the 3D model of the plurality of pre-manufactured placebos, and the plurality of pre-manufactured placebos may be coated by depositing multiple thin film layers of carrier drops 10 (10a, 10b . . . ) on top of each other, so that the plurality of pre-manufactured placebos is coated with the multiple thin film layers of the APIs and/or the excipients, and a variable dosing of the APIs and/or the excipients may be achieved across the multiple thin film layers applied on the plurality of pre-manufactured placebos. In at least some example embodiments of the device, the carrier drops may include high loaded APIs at the time of dispensing. All of the drops are dispensed. The stream 33 may be continuous or discontinuous.

In at least some example embodiments of the device, at least two liquid dispensers are arranged in an array, such as a linear array, a circular array, a mesh array or any other suitable type of array comprising two or more liquid dispensers arranged in an ordered manner. In the example embodiment of the device as depicted in FIG. 1, the at least two liquid dispensers 2,3 are arranged in a first array 11, which is the linear array. The stream 33 of the carrier drops 10 (10a, 10b . . . ) has a trajectory that passes by the at least two liquid dispensers 2,3 configured to dispense drops towards the trajectory of at least the carrier drop 10 in a synchronised manner, so that the carrier drop 10 sequentially collides with the drops that aggregately comprise the subset of the APIs and/or the excipients from and are dispensed by some of the at least two liquid dispensers 2,3, which may be arranged in the array 11.

In at least some example embodiments of the device, such as depicted in FIG. 2, at least some of liquid dispensers 2-9 in the plurality 44 of liquid dispensers 1-9 are arranged in at least the first array 11 and a second array 12, wherein the first array 11 and the second array 12 are configured to dispense drops towards a single stream 33 of the carrier drops 10 (10a, 10b . . . ). In at least some example embodiments, at least the first array 11 is configured to dispense drops towards a first stream 33 of carrier drops 10 (10a, 10b . . . ), and at least the second array 12 is configured to dispense drops towards at least a second stream of carrier drops. Two or more liquid dispensers in the plurality 44 may be configured to dispense two or more separate streams 33 of carrier drops 10 (10a, 10b . . . ).

In at least some example embodiments of the device, the device comprises a mechanisation 22 (FIG. 1) being configured to move the support surface 23 in at least one direction relative to the plurality 44, and/or to move the plurality 44 of liquid dispensers in at least one direction relative to the support surface 23, so that the carrier drops 10 (10a, 10b . . . ) in the stream 33 land in the select XY coordinates on the plurality of pre-manufactured placebos located on the support surface 23.

In at least some example embodiments of the device, the device comprises a mixing chamber 50 (FIG. 2) which is a partially or a fully closed compartment of the device wherein the plurality 44 of liquid dispensers and the support surface 23 are positioned, and the mixing chamber 50 is the fully closed compartment during the time in which the drops are in-flight.

In at least some example embodiments of the device, the device comprises electric plates 21 (FIG. 4) arranged perpendicularly to a path of at least one drop dispensed from at least one liquid dispenser 1, wherein the electric plates 21 have holes through which the at least one drop passes, the at least one liquid dispenser 1 is configured to provide an electric charge to the at least one drop, and the electric plates 21 are configured so that the electric plates 21 exert electric fields on the at least one drop in a synchronised manner, so that the at least one drop is manipulated with the electric fields exerted by the electric plates 21 during the time in which the at least one drop passes through the electric fields exerted by the electric plates 21. In at least some example embodiments of the device, the electric plates 21 are mounted perpendicularly to the trajectory of the carrier drops 10 (10a, 10b . . . ).

Hereinafter at least some example embodiments of the method for applying APIs and/or excipients on pre-manufactured placebos(hereinafter: the method) are described. In at least some example embodiments of the method, a user provides user data, including but not limited to a3D model of the plurality of pre-manufactured placebos to be coated with APIs or onto which the APIs and/or excipients are applied, a coating resolution, and other relevant user data. After the user data is provided by the user, a control unit 1000 may generate the control signals, which may be used to control the device. In at least some example embodiments, the method comprises selecting at least some select XY coordinates on the 3D model of the plurality of pre-manufactured placebos, by using the control unit 1000, and selecting one subset of the APIs and/or the excipients from the set for each of the select XY coordinates, by using the control unit 1000, so that the APIs and/or the excipients may be applied in the select XY coordinates on the plurality of pre-manufactured placebos, and each of the select XY coordinates has one subset of the APIs and/or the excipients selected. In the next step, the user may give the signal to start the device.

In at least some example embodiments of the method for applying APIs and/or excipients on pre-manufactured placebos disclosed herein, the method including dispensing and colliding drops, by using a plurality 44 of at least liquid dispensers 1-3, so that a stream 33 of carrier drops 10 (10a, 10b . . . ) is dispensed and each of the carrier drops 10 (10a, 10b . . . ) in the stream 33 collides with drops that aggregately comprise one subset of APIs and/or excipients from a set, so that a pre-determined amount and combination of APIs and/or excipients is located in each of the carrier drops (10a, 10b . . . ) during flight, and configuring a support surface 23, so that the each of the carrier drops 10 (10a, 10b . . . ) lands on one of select XY coordinates on a plurality of pre-manufactured placebos positioned on the support surface 23. When all of the carrier drops 10 (10a, 10b . . . ) in the stream 33 land on all of the select XY coordinates on the plurality of pre-manufactured placebos positioned on the support surface 23, such a correct depositing of the carrier drops 10 (10a, 10b . . . ) in all of the select XY coordinates results in the APIs and/or the excipients applied on the plurality of pre-manufactured placebos positioned on the support surface 23. Main steps of the method are schematically depicted in FIG. 6. In at least some example embodiments of the method, the method includes using any one of the devices according to the present disclosure. After the APIs and/or the excipients have been applied on the plurality of pre-manufactured placebos on the support surface 23 using at least some example embodiments of the devices and methods hereof, the plurality of pre-manufactured placebos is removed from the device. At least some example embodiments of the method include using any one of the example embodiments of the device as disclosed herein.

At least some example embodiments according to the present disclosure may relate to a product of the device and the method for applying APIs and/or excipients on pre-manufactured placebos according to the present disclosure. In at least some example embodiments of the product of the present disclosure, the product comprises a plurality of pre-manufactured placebos having APIs and/or excipients applied by using any one of the devices and any one of the methods for applying APIs and/or excipients on pre-manufactured placebos according to the present disclosure.

At least some example embodiments according to the present disclosure relate to a device and a method for applying active pharmaceutical ingredients (hereinafter: APIs) and/or excipients on medical devices. Hereinafter at least some example embodiments of devices and methods that relate to the device and the method for applying APIs and/or excipients on medical devices are described in more detail. In at least some example embodiments, the device for applying APIs and/or excipients on medical devices (hereinafter: the device) is configured to apply APIs and/or excipients on a medical device by using the method for applying APIs and/or excipients on medical devices (hereinafter: the method) according to the present disclosure. The medical device may be any appropriate medical device onto which APIs and/or excipients may be applied, such as: an implant, a micro-needle patch, or any other medical device, which may be coated with APIs and/or excipients. In at least some example embodiments, the device and the method are used to coat the medical device with a set of APIs and/or excipients. The set comprises APIs and/or excipients, such as: APIs, polymers (such as: hydroxypropyl methyl cellulose (HPMC), poly vinyl alcohol (PVA), Eudragit® derivates, polyvinylpyrroldone (PVP), polyethylene glycol (PEG), Soluplus®), surfactants (such as: polysorbate 20—Tween 20®, polysorbate 80—Tween 80® or other), liquid ink carriers (such as: water, ethanol, propanol, dimethyl sulfoxide (DMSO) or any combination of liquid ink carriers), humectants (such as: PEG, glycerol, or other), viscosity modifiers (such as: glycerol, PEG, or other), sustained release agents, modified release agents, or other APIs and/or excipients, which may be applied to the medical device. The medical device is coated, by using the device and the method, with the set of APIs and/or excipients. The device comprises a plurality 44 of liquid dispensers. In an example embodiment of the device as depicted in FIG. 1, the plurality 44 of liquid dispensers comprises liquid dispensers 1-3.

The set of APIs and/or excipients is held in a plurality of containers in at least some of the liquid dispensers in the plurality 44 comprising of at least liquid dispensers 1-3 in the device. Each of the APIs and/or excipients from the set may be suspended at a known concentration in a separate liquid, which is held in a separate container in at least one liquid dispenser in the plurality 44 of liquid dispensers in the device. In at least some example embodiments of the device, the device comprises a user interface configured to provide user data, which may include a 3D model of the medical device to be coated using the device disclosed herein, a coating resolution, layers of coating, and other relevant user data that relate to applying APIs and/or excipients on the medical device. In at least some example embodiments of the device and the method, the device comprises a control unit 1000, which is configured to select a series of select XYZ coordinates on a surface of the 3D model of the medical device, so that the series of select XYZ coordinates correspond to select coordinates on the surface of the medical device whereat APIs and/or excipients will be applied by using the device and the method disclosed herein. The device comprises a support surface 23. The medical device to be coated with the APIs and/or the excipients, by using the devices and the methods disclosed herein, is positioned on the support surface 23. In at least some example embodiments, the device comprises the control unit 1000 being configured to assign one subset of APIs and/or excipients from the set to each of the select XYZ coordinates in the series of select XYZ coordinates. The subset of APIs and/or excipient, which is assigned to the each of the select XY coordinates, includes APIs and/or excipients from the set, which are to be applied at the each of the select XYZ coordinates on the medical device. All of the select XYZ coordinates have at least one subset of APIs and/or excipients from the set assigned. After all subsets of APIs and/or excipients from the set are assigned to all of the select XY coordinates, the user may provide a signal to start the device via the user interface. The control unit 1000 may be configured to generate control signals, which are used to control the device so that the medical device is coated with the APIs and/or the excipients from the set.

In at least one example embodiment of the device, such as depicted in FIG. 1, the device comprises the plurality 44 of liquid dispensers 1-3 being configured to dispense drops, wherein a stream 33 of carrier drops 10 (10a, 10b . . . ) is dispensed, and each of the carrier drops 10 (10a, 10b . . . ) in the stream 33 sequentially collides with a pre-determined number of the drops that aggregately comprise one subset of APIs and/or excipients selected from the set, so that a pre-determined amount and combination of APIs and/or excipients is located in each of the carrier drops 10 (10a, 10b . . . ) in the stream 33 during flight; and the support surface 23 being configured so that the each of the carrier drops 10 (10a, 10b . . . ) in the stream 33 lands on one of the select XYZ coordinates on the medical device positioned on the support surface 23. In at least some example embodiments of the device, each of the liquid dispensers in the plurality 44 comprising of at least liquid dispensers 1-3 comprises at least a container for holding liquids to dispense, a drop generating element positioned and configured to cause at least one drop to dispense, a nozzle through which the at least one drop is ejected from, a capillary which connects the container with the nozzle so that the liquid runs from the container through the capillary and out through the nozzle for the at least one drop to be dispensed, and a synchronisation means, which enable each of the liquid dispensers to operate in a synchronised manner with other liquid dispensers in the plurality 44 of liquid dispensers. In some example embodiments of the device, a single container is configured to supply liquids to two or more nozzles in the plurality 44 of liquid dispensers. In some example embodiments of the device, two or more containers are configured to supply liquids to a single nozzle in the plurality 44 of liquid dispensers. At least one liquid dispenser, such as the liquid dispenser 1 in FIG. 1, is configured to dispense the carrier drops 10 (10a, 10b . . . ) in the stream 33, wherein the stream 33 comprises a first carrier drop, a second carrier drop 10a, and at least a third carrier drop 10b, and the plurality 44 of liquid dispensers is configured so that the first carrier drop collides with at least drops that aggregately comprise a first subset of APIs and/or excipients, the second carrier drop 10a collides with at least drops that aggregately comprise a second subset of APIs and/or excipients, the third carrier drop 10b collides with at least drops that aggregately comprise a third subset APIs and/or excipients from, and so on, until all of the carrier drops 10 (10a, 10b . . . ) in the stream 33 collide with at least some drops that aggregately comprise at least some appropriate APIs and/or excipients from the set, and all of the carrier drops 10 (10a, 10b . . . ) in the stream 33 land on the support surface 23. The first subset may, for example, comprise of APIs and/or excipients such as myocites, growth factors, or biopolymer gels, the second subset may, for example, comprise of APIs and/or excipients such as vascular endothelial cells, growth factors, or biopolymer gels, the third subset may comprise of APIs and/or excipients such as scaffolding material, growth factors, or biopolymer gels; a fourth subset may comprise of other biomaterials, so that the medical device may be coated in a highly complex manner. The first subset may be dispensed in, for example, five (5) drops, by using five (5) liquid dispensers from the plurality 44, which may include, for example, thirty (30) liquid dispensers in the device, and all of the five (5) drops collide and merge with the first carrier drop 10 in the stream 33 during flight. The second subset may be dispensed in, for example, seven (7) drops, by using seven (7) liquid dispensers from the plurality 44 of (in this given example) the thirty (30) liquid dispensers in the device, and all of the seven (7) drops collide and merge with the second carrier drop 10a in the stream 33 during flight. Similarly, the third subset may be dispensed in any appropriate number of drops that collide and merge with the third carrier drop 10b, and so on. Each subset of APIs and/or excipients is dispensed in one plurality of drops, which is dispensed from one appropriate sub-plurality of liquid dispensers (in the plurality 44 of liquid dispensers in the device) and collides and merges with one carrier drop 10. Collisions occur between each of the carrier drops 10 (10a, 10b . . . ) in the stream 33 and all drops that carry APIs and/or excipients from any one appropriate subset of APIs and/or excipients from the set assigned to the each of the carrier drops 10 (10a, 10b . . . ). It is understood that any appropriate combinations of APIs and/or excipients may be included in the set, or in any of the subsets. In at least some example embodiments, the series of select XYZ coordinates includes multiple layers on the surface of the 3D model of the medical device, and the medical device may be coated by depositing multiple thin film layers of carrier drops 10 (10a, 10b . . . ) on top of each other, so that the medical device is coated with the multiple thin film layers of APIs and/or excipients, and a variable dosing of APIs and/or excipients may be achieved across the multiple thin film layers applied on the medical device.

All of the drops are dispensed. The stream 33 may be continuous or discontinuous.

In at least some example embodiments of the device, the carrier drops comprise high loaded APIs at the time of dispensing.

In at least some example embodiments of the device, at least two liquid dispensers are arranged in an array, such as a linear array, a circular array, a mesh array or any other suitable type of array comprising two or more liquid dispensers arranged in an ordered manner. In the example embodiment of the device as depicted in FIG. 1, the at least two liquid dispensers 2,3 are arranged in a first array 11, which is the linear array. The stream 33 of the carrier drops 10 (10a, 10b . . . ) has a trajectory that passes by the at least two liquid dispensers 2,3 configured to dispense drops towards the trajectory of at least the carrier drop 10 in a synchronised manner, so that the carrier drop 10 sequentially collides with the drops that aggregately comprise the subset of APIs and/or excipients from and are dispensed by some of the at least two liquid dispensers 2,3, which may be arranged in the array 11.

In at least some example embodiments of the device, such as depicted in FIG. 2, at least some of liquid dispensers 2-9 in the plurality 44 of liquid dispensers 1-9 are arranged in at least the first array 11 and a second array 12, wherein the first array 11 and the second array 12 are configured to dispense drops towards a single stream 33 of the carrier drops 10 (10a, 10b . . . ). In at least some example embodiments, at least the first array 11 is configured to dispense drops towards a first stream 33 of carrier drops 10 (10a, 10b . . . ), and at least the second array 12 is configured to dispense drops towards at least a second stream of carrier drops. Two or more liquid dispensers in the plurality 44 may be configured to dispense two or more separate streams 33 of carrier drops 10 (10a, 10b . . . ).

In at least some example embodiments of the device, the device comprises a mechanisation 22 (FIG. 1) being configured to move the support surface 23 in at least one direction relative to the plurality 44 of liquid dispensers, and/or to move the plurality 44 of liquid dispensers in at least one direction relative to the support surface 23, so that the carrier drops 10 (10a, 10b . . . ) in the stream 33 land in the series of select XYZ coordinates on the medical device positioned on the support surface 23.

In at least some example embodiments of the device, the device comprises a mixing chamber 50 (FIG. 2), which is a partially or a fully closed compartment of the device wherein the plurality 44 of liquid dispensers and the support surface 23 are positioned, and the mixing chamber 50 is the fully closed compartment during the time in which the drops are in-flight. In the mixing chamber 50 at least one environment regulator may be positioned, and the environment regulator may be configured to regulate at least a temperature and a humidity in the mixing chamber 50 over time.

In at least some example embodiments of the device, the device comprises electric plates 21 (FIG. 4) arranged perpendicularly to a path of at least one drop dispensed from at least one liquid dispenser 1, wherein the electric plates 21 have holes through which the at least one drop passes, the at least one liquid dispenser 1 is configured to provide an electric charge to the at least one drop, and the electric plates 21 are configured so that the electric plates 21 exert electric fields on the at least one drop in a synchronised manner, so that the at least one drop is manipulated with the electric fields exerted by the electric plates 21 during the time in which the at least one drop passes through the electric fields exerted by the electric plates 21. In at least some example embodiments of the device, the electric plates 21 are mounted perpendicularly to the trajectory of the carrier drops 10 (10a, 10b . . . ).

Hereinafter at least some example embodiments of the method for applying APIs and/or excipients on medical devices (hereinafter: the method) are described in more detail. In at least some example embodiments of the method, a user provides user data, including but not limited to the 3D model of the medical device to be coated, a coating resolution, and other relevant user data. After user data is provided by the user via a user interface, a control unit 1000 may generate the control signals, which may be used to control the device. In at least some example embodiments, the method comprises selecting a series of select XYZ coordinates on a surface of the 3D model of the medical device, by using the control unit 1000, and assigning one subset of APIs and/or excipients from a set to each of the select XYZ coordinates in the series of the select XYZ coordinates, by using the control unit 1000, so that APIS and/or excipients may be applied in the series of the select XYZ coordinates on the medical device, and each of the select XYZ coordinates has one subset of APIs and/or excipients assigned. In the next step, the user may give the signal to start the device. In at least some example embodiments of the method for bioprinting disclosed herein, the method includes dispensing and colliding drops by using at least a plurality of liquid dispensers 1-3, so that a stream 33 of carrier drops 10 (10a, 10b . . . ) is dispensed and each of the carrier drops 10 (10a, 10b . . . ) collides and merges with at least drops that aggregately comprise one subset of APIs and/or excipients from the set during flight, and collecting the carrier drops 10 (10a, 10b . . . ), by using a support surface 23, in a series of select XYZ coordinates on a medical device located on the support surface 23, so that at least one subset of APIs and/or excipients is applied on the medical device. Main steps of the method are schematically depicted in FIG. 6. When all of the carrier drops 10 (10a, 10b . . . ) in the stream 33 land on all of the select XYZ coordinates on the medical device positioned on the support surface 23, such a correct depositing of the carrier drops 10 (10a, 10b . . . ) in all of the select XYZ coordinates results in the APIs and/or excipients applied on the medical device positioned on the support surface 23. In at least some example embodiments of the method, the method includes using any one of the devices according to the present disclosure. After the APIs and/or excipients have been applied on the medical device on the support surface 23 using at least some example embodiments of the devices and methods hereof, the medical device is removed from the device. At least some example embodiments of the method include using any one of the example embodiments of the device as disclosed herein.

At least some example embodiments according to the present disclosure relate to a product of the device and the method for applying APIs and/or excipients on medical devices according to the present disclosure. In at least some example embodiments of the product of the present disclosure, the product is a medical device having APIs and/or excipients applied/coated by using any one of the devices and the methods for applying APIs and/or excipients on medical devices according to the present disclosure.

At least some example embodiments according to the present disclosure relate to a device and a method for assembly of nucleic acids for pharmaceutical, phytopharmaceutical or veterinary use. Hereinafter at least some example embodiments of devices and methods that relate to the device and the method for assembly of the nucleic acids for pharmaceutical, phytopharmaceutical or veterinary use are described in more detail. In at least some example embodiments, the device for assembly of nucleic acids for pharmaceutical, phytopharmaceutical or veterinary use (hereinafter: the device) is used to produce final nucleic acid molecules for pharmaceutical, phytopharmaceutical or veterinary use, by using the method for assembly of the nucleic acids for pharmaceutical, phytopharmaceutical or veterinary use (hereinafter: the method). In at least some example embodiments, the device and the method are used to manufacture final nucleic acid molecules by assembling (hybridising) two or more nucleic acid components (hereinafter: components) from a set of nucleic acid components. Components may be either naturally occurring or artificially synthesised nucleic acids, such as: oligonucleotides, DNA fragments, plasmids, TNA, peptides, RNA fragments, small metabolites, or any other either organic or inorganic nucleic acids or any combination of components listed herein. Components in the set may have hybridisation regions, which may be complementary single-stranded overhangs or similar, and the hybridisation regions enable components to assemble and bind with other components from the set. The term ‘assemble’ and/or ‘assemble and bind together” shall herein refer to a process of hybridization of nucleic acid components together. The set comprises any number and/or combination of any appropriate components from which final nucleic acids for pharmaceutical, phytopharmaceutical or veterinary use may be manufactured using the device and the method. The final nucleic acid molecules are manufactured, by using the device and the method, from the set of components. Each of the final nucleic acid molecules comprises two or more components from the set assembled and bound together. In at least some example embodiments of the device, such as depicted in FIG. 1, the device comprises a plurality 44 of liquid dispensers 1-3 being configured to dispense drops, wherein at least a carrier drop 10 sequentially collides with at least drops that aggregately comprise a subset of nucleic acids from the set, so that the subset of nucleic acids is located in the carrier drop 10, wherein the subset of nucleic acids comprises all components to produce final nucleic acid molecules, and a support surface 23 configured to collect the carrier drop 10. In at least one preferred example embodiment of the device, the device comprises the plurality 44 including three hundred (300) liquid dispensers.

The set of components is held in a plurality of containers in at least some of the liquid dispensers in a plurality 44 of liquid dispensers, in the device. Each of the components from the set is suspended at a known concentration in a separate liquid, which is held in a separate container in at least one liquid dispenser in the plurality 44 of liquid dispensers in the device. Each of the liquid dispensers in the plurality 44 comprises at least a container for holding liquids to dispense, a drop generating element positioned and configured to cause at least one drop to dispense, a nozzle through which the at least one drop is ejected from, a capillary which connects the container with the nozzle so that the liquid runs from the container through the capillary and out through the nozzle for the at least one drop to be dispensed, and synchronisation means, which enable each of the liquid dispensers to operate in a synchronised manner with other liquid dispensers in the plurality 44 of liquid dispensers. In some example embodiments of the device, a single container is configured to supply liquids to two or more nozzles in the plurality 44 of liquid dispensers. In some example embodiments of the device, two or more containers are configured to supply liquids to a single nozzle in the plurality 44 of liquid dispensers. At least one liquid dispenser, such as the liquid dispenser 1 (FIG. 1) is configured to dispense the carrier drop 10.

In at least some example embodiments of the device, the device comprises at least one liquid dispenser, such as the liquid dispenser 1 in FIG. 1, being configured to dispense a stream 33 of carrier drops 10 (10a, 10b . . . ), wherein the stream 33 comprises a first carrier drop 10, a second carrier drop 10a, and at least a third carrier drop 10b, and the plurality 44 of liquid dispensers may be configured so that the first carrier drop 10 collides and merges with at least drops that aggregately comprise a first subset of components, the second carrier drop 10a collides and merges with at least drops that aggregately comprise a second subset of components, the third carrier drop 10b collides and merges with at least drops that aggregately comprise a third subset components, and so on, until all of the carrier drops 10 (10a, 10b . . . ) in the stream 33 collide and merge with at least some drops that aggregately comprise at least some appropriate components from the set, and all of the carrier drops 10 (10a, 10b . . . ) in the stream 33 land on the support surface 23. The first subset may be dispensed in, for example, five (5) drops, by using five (5) liquid dispensers from the plurality 44 of, for example, thirty (30) liquid dispensers in the device, and all of the five (5) drops collide and merge with the first carrier drop 10 in the stream 33 during flight. The second subset may be dispensed in, for example, seven (7) drops, by using seven (7) liquid dispensers from the plurality 44 of (in this given example) the thirty (30) liquid dispensers in the device, and all of the seven (7) drops collide and merge with the second carrier drop 10a in the stream 33 during flight. Similarly, the third subset may be dispensed in any appropriate number of drops that collide and merge with the third carrier drop 10b, and so on. Each subset of components is dispensed in one plurality of drops, which is dispensed from one appropriate sub-plurality of liquid dispensers (from the plurality 44 of liquid dispensers in the device) and collides and merges with one carrier drop 10. Collisions occur between each of the carrier drops 10 (10a, 10b . . . ) in the stream 33 and all drops that carry components from any one appropriate subset of components from the set assigned to the each of the carrier drops 10 (10a, 10b . . . ). It is understood that any appropriate combinations of components may be included in the set, or in any of the subsets.

In at least some example embodiments of the device, at least two liquid dispensers are arranged in an array, such as a linear array, a circular array, a mesh array or any other suitable type of array comprising two or more liquid dispensers arranged in an ordered manner. In the example embodiment of the device as depicted in FIG. 1, the at least two liquid dispensers 2,3 are arranged in a first array 11, which is the linear array. Each of the carrier drops 10 (10a, 10b, , , ) has a trajectory that passes by the at least two liquid dispensers 2,3 configured to dispense drops towards the trajectory of the carrier drop 10 in a synchronised manner, so that the carrier drop 10 sequentially collides with the drops that aggregately comprise components from the subset and are dispensed by the at least two liquid dispensers 2,3, which may be arranged in the array 11. In a preferred example embodiment of the device, the device comprises the linear array of five hundred (500) liquid dispensers and the carrier drop 10 passes by all five hundred nozzles in the linear array in close proximity.

In at least some example embodiments of the device as depicted in FIG. 2, at least some of liquid dispensers 2-9 in the plurality 44 of liquid dispensers 1-9 are arranged in at least the first array 11 and a second array 12, wherein the first array 11 and the second array 12 are configured to dispense drops towards a single stream 33 of the carrier drops 10 (10a, 10b . . . ). In at least some example embodiments, at least the first array 11 is configured to dispense drops towards a first stream 33 of carrier drops 10 (10a, 10b . . . ), and at least the second array 12 is configured to dispense drops towards a second stream of carrier drops. Two or more liquid dispensers in the plurality 44 may be configured to dispense two or more separate streams 33 of carrier drops 10 (10a, 10b . . . ).

In at least some preferred example embodiments of the device, each of the liquid dispensers in the plurality 44 of liquid dispensers is configured to dispense up to a hundred thousand (100000) drops per second. In at least some preferred example embodiments of the device, each of the liquid dispensers in the plurality 44 of liquid dispensers is configured to dispense drops with a volume in a range between 1 picoliter to 100 nanoliters. In at least some preferred example embodiments of the device, the device comprises fifty or more, or five hundred or more, or ten thousands or more liquid dispensers in the plurality of liquid dispensers.

In at least some example embodiments of the device, the support surface 23 is made of a bendable material or a non-bendable material. In at least some example embodiments of the device, the support surface 23 is detachable or non-detachable relative to the device. In at least some example embodiments of the device, the support surface 23 is a container filled with a liquid in which the carrier drops land. In at least some example embodiments of the device, the support surface 23 is a flat surface. In at least some example embodiments of the device, the plurality 44 of liquid dispensers are configured to dispense drops of a first liquid, and the support surface 23 is filled or covered with a second liquid, and the first liquid and the second liquid are immiscible. The first liquid and the second liquid are immiscible and do not mix with each other, and the carrier drops 10 (10a, 10b . . . ) may remain intact, may not merge amongst each other, and may be protected from evaporation immediately after landing in the second liquid, which located in or on the support surface 23.

In at least some example embodiments of the device, the carrier drop 10 includes binding reagents, which enable an assembly and binding of the components located in the carrier drop 10, wherein the binding reagents are included in the carrier drop 10 or provided by a drop that collides and merges with the carrier drop 10 during flight. In at least some example embodiments of the device, the carrier drop 10 includes anti-solvent liquids (such as, but not limited to: water, tert-butanol, ethanol), at the time of dispensing, so that components included in drops that collide with the carrier drops 10 (10a, 10b . . . ), do not solve in the carrier drop 10 comprising the anti-solvent liquids.

In at least some example embodiments, the device comprises a mixing chamber 50 (FIG. 2) which is a partially or a fully closed compartment of the device wherein the plurality 44 of liquid dispensers and the support surface 23 are positioned, and the mixing chamber 50 is the fully closed compartment during a time in which the drops are in-flight. In at least some example embodiments, the device comprises at least one environment regulator positioned in the mixing chamber 50, and the environment regulator is configured to regulate at least a temperature and a humidity in the mixing chamber 50 over time. In a preferred example embodiment, the at least one environment regulator is configured to minimise evaporation of drops during the time in which the drops are in-flight.

In at least some example embodiments of the device, the device comprises electric plates 21 (FIG. 4) arranged perpendicularly to a path of at least one drop dispensed from at least one liquid dispenser 1, wherein the electric plates 21 have holes through which the at least one drop passes, the at least one liquid dispenser 1 is configured to provide an electric charge to the at least one drop, and the electric plates 21 are configured so that the electric plates 21 exert electric fields on the at least one drop in a synchronised manner, so that the at least one drop is manipulated with the electric fields exerted by the electric plates 21 during the time in which the at least one drop passes through the electric fields exerted by the electric plates 21. In at least some example embodiments of the device, the electric plates 21 are mounted perpendicularly to the trajectory of the carrier drops 10 (10a, 10b . . . ).

In at least some example embodiments of the device, the device comprises a processing chamber 60 (FIG. 3) having at least one processing element 401 configured to change a temperature in the processing chamber 60 over time so that a sequence of temperatures is achieved over time in the processing chamber 60, and the sequence of temperatures causes a biochemical reaction to occur between the components and binding reagents located in each carrier drop 10 positioned in the processing chamber 60, wherein the biochemical reaction causes the components located in each of the carrier drops to assemble and bind together to form the final nucleic acid molecules, wherein the biochemical reaction is either Restriction/Ligation (sticky end ligation or blunt end ligation), or a Polymerase Chain Reaction (hereinafter: PCR), or a Cell Free Cloning, or a BioBricks Assembly, or a Gibson Assembly, or a HiFi Assembly, or in vivo cloning (homologous recombination), or any other appropriate biochemical reaction that enables assembly and binding together of the components to form the final nucleic acid molecules. At least some of the biochemical reactions include multiplication of the final nucleic acid molecules. The sequence of temperatures comprises at least a first temperature held for a pre-determined duration of time in the processing chamber 60, a second temperature held for a pre-determined duration of time in the processing chamber 60, and so on, until all temperatures are achieved in the sequence of temperatures and the biochemical reaction occurs successfully in each of the carrier drops 10 (10a, 10b . . . ) positioned in the processing chamber 60.

In at least some example embodiments wherein Restriction/Ligation (sticky end ligation or blunt end ligation) is the biochemical reaction used with the device and the method to assembly and bind the components into the final nucleic acid molecules, the set may include components such as a plurality of combinatorial pre-prepared oligonucleotides with specifically predefined beginnings and ends of such oligonucleotides, and the binding reagents may include restriction endonuclease solution or ligase enzyme solution, buffer, bovine serum albumin (BSA) or other liquids and components.

In at least some example embodiments wherein the PCR is the biochemical reaction used with the device and the method to assemble and bind together the components into the final nucleic acid molecules, the set may include components such as a plurality of combinatorially pre-prepared oligonucleotides having complementary single-stranded overhangs added onto the beginnings and ends of such oligonucleotides, and the binding reagents may include DNA polymerase solution, dNTPs solution, reaction buffer, MgCL2 solution or other liquids or components.

In at least some example embodiments wherein the in vivo cloning (homologous recombination) is the biochemical reaction used with the device and the method to assemble and bind together the components into the final nucleic acid molecules, the set may include components such as: modified E. coli cells capable of in vivo cloning, yeast cells capable of in vivo cloning, cell-carrying liquid (such as hydrogel or other), various nucleic acid fragments, various plasmids, buffers, growth medium or other liquids or components.

In at least some example embodiments wherein the Cell Free Cloning is the biochemical reaction used with the device and the method to assemble and bind together the components into the final nucleic acid molecules, the set may include components such as: various nucleic acid fragments and various plasmids, water, predefined solutions, or other, and the binding reagents may include various enzymes mimicking bacterial replication mechanisms, buffers, or other liquids or components.

In at least some example embodiments wherein the Biobricks is the biochemical reaction used with the device and the method to assemble and bind the components into the final nucleic acid molecules, the set may include components such as: various nucleic acid fragments encoding for promoter, ribosomal binding site (RBS), coding sequences and or terminators, various plasmids, various enzymes or other liquids or components.

In at least some example embodiments wherein Golden gate assembly or the Gibson assembly is the biochemical reaction used with the device and the method to assemble and bind together the components into the final nucleic acid molecules, the set may include components such as: different vectors, DNA fragments, buffer, different enzymes such as exonuclease, DNA polymerase and DNA ligase etc. as well as other relevant cloning liquids or components.

In at least some example embodiments wherein the Hifi Assembly is the biochemical reaction used with the device and the method to assemble and bind together the components into the final nucleic acid molecules, the set may include components such as: multiple DNA fragments, high fidelity polymerase, buffer and other relevant liquids or components.

In at least some example embodiments of the device, the device comprises a mechanisation 22 (FIG. 1) being configured to move the support surface 23 in at least one direction relative to the plurality 44 of liquid dispensers, and/or to move the plurality 44 of liquid dispensers in at least one direction relative to the support surface 23, so that the carrier drops 10 (10a, 10b . . . ) in the stream 33 may land on select XY coordinates on the support surface 23.

Hereinafter at least some example embodiments of the method for assembly of nucleic acids are described in more detail. In at least some example methods, a user inputs the user data, including but not limited to a type of final nucleic acid molecules to be manufactured using the device and the method, device settings, method setting, biochemical reaction settings, and other relevant user data. After the user data is provided by the user, a control unit 1000 may generate the control signals, which may be used to control the device so that final nucleic acid molecules are manufactured from two or more components from a set of nucleic acid components. In at least some example embodiments of the method, the method comprises creating a model of the final nucleic acid molecules to be produced using the device, by using the control unit 1000. In at least some example embodiments of the method, the method includes selecting at least some select XY coordinates on a support surface 23. In the next step, the user may give the signal to start the device. In at least some example embodiments of the method for assembly of nucleic acids disclosed herein, the method includes dispensing, by using a plurality 44 of at least liquid dispensers 1-3, at least one carrier drop and at least drops that aggregately comprise a subset of components from a set of nucleic acid components, wherein the carrier drop 10 sequentially collides with at least drops that aggregately comprise the subset of components, so that the subset of components is located in the carrier drop during flight, and collecting at least the carrier drop 10 by using the support surface 23. Main steps of the method are schematically depicted in FIG. 6. A stream 33 of carrier drops 10 (10a, 10b . . . ) may be dispensed. In at least some example embodiments of the method, the method includes using any one of the devices according to the present disclosure. When all of the carrier drops 10 (10a, 10b . . . ) in the stream 33 land on the support surface 23 at select XY coordinates coordinates, and components in at least the subset are assembled and bound together in a processing chamber 60 by using the biochemical reaction, the combined result of such a correct depositing of components and binding reagents in the carrier drops 10 (10a, 10b . . . ), and assembling and binding of the components together in the processing chamber 60, results in the final nucleic acid molecules manufactured on the support surface 23. After the final nucleic acid molecules are manufactured using at least some example embodiments of the devices and methods hereof, the final nucleic acid molecules are removed from the device. At least some example embodiments of the method include using any one of the example embodiments of the device as disclosed herein.

A detailed description of some features of any aforesaid example embodiments of the devices and the methods is provided hereinafter. The following text may relate to any one of the aforesaid technical solutions. Hereinafter ‘the device’ refers to any of the aforesaid devices, such as the device for mixing liquids, the device for preparing samples, the device for bioprinting of multi-component 3D objects, the device for 3D printing multi-material 3D objects, and/or other aforesaid devices.

In at least some example embodiments of the device, the drop generating element in any of the liquid dispensers in the plurality 44 may be a piezo element. In such example embodiments, the synchronisation means are configured to apply voltage to the piezo element at a pre-determined moment in time, so that the piezo element deforms and pushes the liquid out through the nozzle, and in this way, the at least one drop is dispensed at a correct time and in the synchronised manner with the other liquid dispensers in the plurality 44. The voltage applied to the piezo element determines the deformation of the piezo element and consequently a size of the at least one drop and a speed at which the at least one drop is dispensed.

In at least some example embodiments of the device, the drop generating element in any of the liquid dispensers in the plurality 44 may be a thermal element configured to change the temperature of the liquid used for dispensing drops. In such example embodiments, the synchronisation means are configured to control the thermal element so that the thermal element increases or decreases the temperature of the liquid used for dispensing the at least one drop in a pre-determined moment in time and in the synchronised manner with the other liquid dispensers in the plurality 44. When the synchronisation means control the thermal element so that the thermal element causes the temperature of the liquid to increase, said liquid expands in volume and thus ejects out through the nozzle, and in this way, at least one drop is dispensed. The change in temperature and consequently expansion of the volume of the liquid determines the size of the at least one drop and the speed at which the at least one drop is dispensed.

In at least some example embodiments of the device, the drop generating element in any of the liquid dispensers in the plurality 44 may be a solenoid actuator (or valve) configured to produce a mechanical movement which pushes the liquid through the nozzle for the at least one drop to be dispensed. In such example embodiments, the synchronisation means are configured to control the solenoid actuator so that the solenoid actuator produces the mechanical movement which pushes the liquid out through the nozzle in a pre-determined moment in time and in the synchronised manner with the other liquid dispensers in the plurality 44. When the synchronisation means control the solenoid actuator so that the thermal element causes the temperature of the liquid to increase, said liquid expands in volume and thus ejects out through the nozzle, and in this way, at least one drop is dispensed.

In at least some example embodiments of the device, the drop generating element in any of the liquid dispensers in the plurality 44 may be any other electro-mechanical part capable of producing a mechanical movement that pushes the liquid out through the nozzle, or any other thermal element configured to change the temperature of the liquid so that the liquid expands and is thus ejected through the nozzle.

In at least some example embodiments of the device, the device comprises the mechanisation 22 (FIG. 1). The mechanisation 22 may include any number and combination of electro-mechanical, mechanical, or software parts, that enable a mechanical movement in at least one direction. Parts that are included in the mechanisation 22 may be parts, such as: electrical motors (such as stepper motors or other motors), belts (such as GT2 belt or other belts), couplers, bearings, gears, shafts, rods, linear actuators, pneumatic actuators, guides (such as linear motion guides or other guides), rods, rails (such as linear rails or other rails), conveyor belts, control signal generators (such as PWM signal generators or other control signal generators), application specific software (such as firmware), microcontrollers, any combination of items listed herein, or any other electro-mechanical, mechanical, or software parts, which are capable of moving the support surface 23 or the plurality 44 of liquid dispensers in at least one direction in the device.

In at least some example embodiments of the devices, such as depicted in FIG. 2, the first array 11 and the second array 12 are facing each other so that an angle between the first array 11 and the second array 12 is a hundred and eighty degrees (1800) observing from above. In at least some example embodiments of the devices according to the present disclosure, the first array 11 and the second array 12 may be positioned at a ninety degree (900) angle, a hundred and twenty degree (120°) angle, forty-five (450) degree angle or any other suitable angle. In at least some example embodiments of the device, the first array 11 is configured to dispense drops towards the first stream 33 of carrier drops 10, and the second array 12 is configured to dispense drops towards the second stream of carrier drops. In such example embodiments, the first array 11 and the second array 12 may be positioned in parallel to each other and two separate liquid dispensers in the plurality 44 are configured to dispense two separate streams 33 the carrier drops 10 (10a, 10b . . . ).

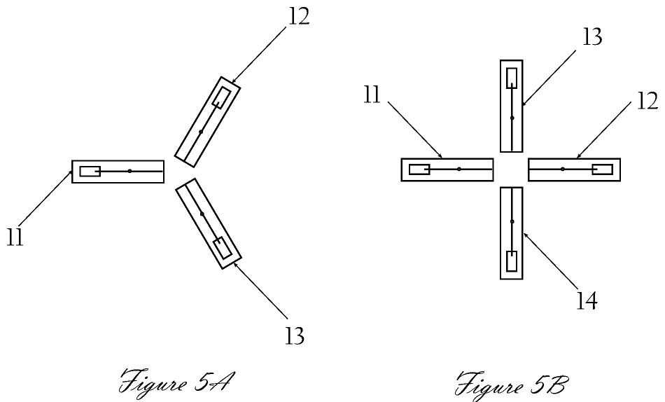

In at least some example embodiments of the device, the device comprises at least some liquid dispensers in the plurality 44 arranged in three (3) linear arrays of liquid dispensers, such as the first array 11, the second array 12 and a third array 13, as depicted schematically in a top view of the three (3) linear arrays in FIG. 5A. All three linear arrays 11,12,13 in FIG. 5A dispense drops towards a single stream 33 of carrier drops 10 (10a, 10b . . . ).

In at least some example embodiments of the device, the device comprises at least some liquid dispensers in the plurality 44 arranged in four (4) linear arrays of liquid dispensers, such as the first array 11, the second array 12, the third array 13, and a fourth array 14, as depicted schematically in the top view of the four (4) linear arrays in FIG. 5B. All of the four (4) linear arrays 11,12,13,14 in FIG. 5B dispense drops towards a single stream 33 of carrier drops 10 (10a, 10b . . . ).

In at least some example embodiments of the device, the device comprises the user interface being configured to provide user data. The user interface comprises any number of electro-mechanical, mechanical or software based user interface parts, which are available now or which shall become available in the future, such as: a display, a touch screen, a computer keyboard, a touch sensor of any kind, knobs, buttons, a camera, a computer, a body sensor, devices for visually or otherwise impaired, a computer mouse, or other. The user interface allows the user of the device to provide the user data.

In at least some example embodiments of the device, the device includes the control unit 1000. The control unit 1000 may comprise at least: one computer which may comprise all standard computer parts such as, but not limited to: at least one data processor, at least one memory storage, at least one data bus, at least one data input and output connecting means, all software parts (such as application-specific software including firmware) and other. In at least some example embodiments, the control unit 1000 may include parts such as: a power storage, a power supply, at least one micro-controller, at least one AD/DA converter, at least one power signal converter, at least one power or control signal amplifier, at least one cable, at least one connector for the at least one cable, and may include other electro-mechanical or mechanical parts which are needed to: a) connect the control unit 1000 physically to other parts of the device (such as the plurality 44 of liquid dispensers, the mechanisation 22 or any other herein disclosed parts of the device), and b) connect the control unit 1000 to other device parts (such as the plurality 44 of liquid dispensers, the mechanisation 22, or any other parts of the device as in FIG. 1 or 2) with data connecting means. The control unit 1000 may be configured to generate each and every control signal, which is used for controlling each and every part of the device (such as the plurality 44 of liquid dispensers or other parts of the device).

The control unit 1000 may send each of the generated control signals to a dedicated part of the device (such as any one liquid dispenser in the plurality 44 of liquid dispensers) via the data output connecting means, and when each of the generated control signals is received by the dedicated part of the device, the dedicated part of the device functions according to the control signal sent from the control unit to the dedicated part of the device. The control unit 1000 may be configured to control all drop generating elements in the plurality 44 of liquid dispensers, so that all drop are dispensed at correct moments in time and all collisions (between at least the carrier drop 10 and other drops) occur, and all liquid dispensers in the plurality 44 of liquid dispensers operate in the synchronised manner.

In at least some example embodiments of the device, the device comprises the electric plates 21 (FIG. 4). The electric plates 21 have tiny holes through which the stream 33 of carrier drops 10 (10a, 10b . . . ) may pass. In one example, the electric plates 21 may comprise a multitude of electrodes in the shape of rings positioned around the holes in the electric plates 21. The electric plates 21 or electrodes may be electrically charged so that electrical charge is accumulated in the electric plates 21 or in any of the electrodes in the multitude of electrodes so that a desired electric field is achieved. The electric plates 21 are configured so that the time-pattern of charging or discharging the electric plates 21 or the multitude of electrodes (and thus the presence of the desired electric field) are tightly synchronized with the stream 33 of carrier drops 10 (10a, 10b . . . ) in order to achieve a desired effect wherein the desired effect is that of: stabilisation of the carrier drop 10 trajectory, adjustment the carrier drop 10 trajectory, acceleration the carrier drop 10 trajectory, deceleration the carrier drop 10 trajectory, or any other manipulation of the carrier drop 10 trajectory. The carrier drop 10 trajectory may in this way be stabilised, or adjusted, or the carrier drops 10 may be accelerated or decelerated. Repeatability of the stream 33 of carrier drops 10 (10a, 10b . . . ) may thus be achieved so that the manipulation of the carrier drop 10 trajectory using electric fields exerted from the electric plates 21 enables compensation of the effects of collisions of the carrier drop 10 with other charge-free drops that collide with the carrier drop 10.

In at least one example, each electric plate 21 is made of uniform conductive material. In at least another example, each electric plate 21 is made of three (3) layers of materials: a layer of insulating material stacked between two layers of conductive material, and in such a case, the two conductive plates may be charged with opposite electrical charges which effectively form an electrical dipole. It is understood that depending on the charge of the carrier drops 10 and the desired effect of the electric fields exerted from the electric plates 21 on the carrier drop 10, voltage may either increase between two neighbouring electric plates 21 in the direction of the carrier drop 10 trajectory, or voltage may decrease between two neighbouring electric plates 21 in the direction of the carrier drop 10 trajectory. In this way, in at least some example embodiments of the device according to the present disclosure, the trajectory the carrier drops 10 (10a, 10b . . . ) in the stream 33 may be manipulated by using the electric fields exerted by the electric plates 21 which are mounted perpendicularly to the carrier drop 10 trajectory and have holes through which the carrier drop 10 passes. Electric plates 21 may be controlled by the control unit 1000.

In a preferred example embodiment of the device, the device includes the linear array comprising of three hundred (300) liquid dispensers, and the carrier drop 10 passes by all three hundred nozzles in the linear array in close proximity.

### ITEMS

Item 1 may include a device for mixing liquids, the device comprising a plurality 44 of liquid dispensers 1-3 configured to dispense drops, wherein at least a carrier drop 10 sequentially collides and merges with drops dispensed from a sub-plurality of liquid dispensers, so that liquids from the sub-plurality of liquid dispensers are located in the carrier drop 10, and a support surface 23 being positioned so that the carrier drop 10 lands on the support surface 23.

Item 2 may include the device of item 1 or some other item herein, wherein at least one liquid dispenser 1 is configured to dispense the carrier drop 10, and/or the carrier drop 10 has a trajectory that passes by at least two liquid dispensers 2,3 configured to dispense drops towards the trajectory of the carrier drop 10 in a synchronised manner, so that the carrier drop 10 sequentially collides with at least all drops that include liquids from the sub-plurality of liquid dispensers and are dispensed by at least some of the at least two liquid dispensers 2,3.

Item 3 may include the device of the item 1 or 2 or some other item herein, wherein at least one liquid dispenser 1 is configured to dispense a stream 33 of carrier drops 10 (10a, 10b . . . ), wherein the stream 33 comprises a first carrier drop 10, a second carrier drop 10a, and at least a third carrier drop 10b, and the plurality 44 comprising of at least the liquid dispensers 1-3 is configured so that the first carrier drop 10 collides with drops from a first sub-plurality of liquid dispensers, the second carrier drop 10a collides with drops from a second sub-plurality of liquid dispensers, the third carrier drop 10b collides with drops from a third sub-plurality of liquid dispensers, and so on, until all of the carrier drops 10 (10a, 10b . . . ) in the stream 33 collide with at least some drops dispensed from at least some liquid dispensers from the plurality 44 of liquid dispensers, and all of the carrier drops 10 (10a, 10b . . . ) in the stream 33 land on the support surface 23.

Item 4 may include the device of any one of the preceding items 1-3 or some other item herein, wherein the at least two liquid dispensers 2,3 are arranged in an array, such as a linear array or a circular array; or at least some of the liquid dispensers are arranged in at least a first array 11 and a second array 12, wherein the first array 11 and the second array 12 are configured to dispense drops towards a single stream 33 of the carrier drops 10 (10a, 10b . . . ), or the first array 11 is configured to dispense drops towards a first stream 33 of carrier drops 10 (10a, 10b . . . ), and the second array 12 is configured to dispense drops towards a second stream of carrier drops 10 (10a, 10b . . . ).

Item 5 may include the device of any one of the items 1-4 or some other item herein, wherein each of the liquid dispensers in the plurality 44 of liquid dispensers is configured to dispense up to a hundred thousand (100000) drops per second, and/or is configured to dispense drops with a volume in a range between 1 picoliter to 100 nanoliters; and/or the plurality 44 of liquid dispensers comprises fifty or more, or five hundred or more, or ten thousands or more liquid dispensers.

Item 6 may include the device of any of the items 1-5 or some other item herein, wherein the support surface 23 is: made of a bendable material or a non-bendable material, and/or is detachable or non-detachable relative to the device, and/or is a container filled with a liquid in which the carrier drops 10 (10a, 10b . . . ) lands, and/or is a surface.

Item 7 may include the device of any one of the items 1-6 or some other item herein, wherein the plurality 44 of liquid dispensers are configured to dispense drops of a first liquid, and the support surface 23 is filled or covered with a second liquid, and the first liquid and the second liquid are immiscible.

Item 8 may include the device of any one of the items 1-7 or some other item herein, characterised in the carrier drops 10 (10a, 10b . . . ) includes anti-solvent liquids, such as: water, tert-butanol, ethanol, and liquids in drops that collide the carrier drops 10 (10a, 10b . . . ), do not solve in the carrier drops 10 (10a, 10b . . . ).

Item 9 may include the device of any one of the items 1-8 or some other item herein, wherein the device comprises a mixing chamber 50, which is a partially or a fully closed compartment of the device wherein the plurality 44 of liquid dispensers and at least one support surface 23 are positioned, and the mixing chamber 50 is the fully closed compartment during the time in which the drops are in-flight.

Item 10 may include the device of any of the items 1-9 or some other item herein, wherein the device comprises electric plates 21 arranged perpendicularly to a path of at least one drop dispensed from at least one liquid dispenser, wherein the electric plates 21 have holes through which the at least one drop passes, the at least one liquid dispenser is configured to provide an electric charge to the at least one drop, and the electric plates 21 are configured so that the electric plates 21 exert electric fields on the at least one drop in a synchronised manner, so that the at least one drop is manipulated with the electric fields exerted by the electric plates 21 during the time in which the at least one drop passes through the electric fields exerted by the electric plates 21.

Item 11 may include the device of any one of the items 1-10 or some other item herein, wherein the device comprises a processing chamber 60 having at least one processing element 401 configured to remove the anti-solvent liquids from each carrier drop 10 located in the processing chamber 60 that includes the anti-solvent liquids.

Item 12 may include the device of any one of the items 1-11 or some other item herein, wherein the device comprises a mechanisation 22 configured to move the support surface 23 in at least one direction relative to the plurality 44 of liquid dispensers, and/or move the plurality 44 of liquid dispensers in at least one direction relative to the support surface 23, and/or move the support surface 23 from the mixing chamber 50 to the processing chamber 60.

Item 13 may include the device of any one of the items 1-12 or some other item herein, wherein each of the liquid dispensers in the plurality 44 of liquid dispensers comprises a container for holding liquids to dispense, a drop generating element positioned and configured to cause at least one drop to dispense, a nozzle through which the at least one drop is ejected from, a capillary which connects the container with the nozzle so that the liquid runs from the container through the capillary and out through the nozzle for the at least one drop to be dispensed, and synchronisation means, which enable each of the liquid dispensers to operate in a synchronised manner with other liquid dispensers in the plurality 44 of liquid dispensers; and/or a single container is configured to supply liquids to two or more nozzles in the plurality 44 of liquid dispensers; and/or two or more containers are configured to supply liquids to a single nozzle in the plurality 44 of liquid dispensers.

Item 14 may include a method for mixing liquids, the method comprising of dispensing, by using a plurality 44 comprising of at least liquid dispensers 1-3, at least one carrier drop 10 and at least drops that comprise liquids from a select sub-plurality of liquid dispensers, wherein the carrier drop 10 sequentially collides and merges with the drops that comprise the liquids from the select sub-plurality of liquid dispensers, so that the liquids from the select sub-plurality of liquid dispensers are located in the carrier drop 10 during flight, and collecting the at least one carrier drop by using a support surface 23.

Item 14 may include a method for mixing liquids using the device of any one of the items 1-13 or some other item herein.

Item 146 may include a device for preparing samples for assays, the device comprising: a plurality 44 of liquid dispensers 1-3 being configured to dispense drops, so that at least a carrier drop 10 sequentially collides with N drops that comprise a first substance and M drops that comprise at least a second substance, so that the first substance and the second substance are located at a ratio of interest (N:M) and at an amount of interest (N+M) in the carrier drop 10 during flight, and a support surface 23 being configured to collect the carrier drop 10 on a select XY coordinate on the support surface 23, and in this way, the samples are prepared for an assay on the support surface 23.

Item 17 may include the device of the item 16 or some other item herein, wherein at least one liquid dispenser 1 from the plurality 44 comprising of at least the liquid dispensers 1-3 is configured to dispense the carrier drop 10, and/or the carrier drop 10 has a trajectory that passes by at least two liquid dispensers 2,3 configured to dispense drops towards the trajectory of the carrier drop 10 in a synchronised manner, so that the carrier drop 10 sequentially collides with at least the N drops and the M drops, which are dispensed from at least some liquid dispensers of the at least two liquid dispensers.

Item 18 may include the device of the item 16 or 17 or some other item herein, wherein at least one liquid dispenser 1 is configured to dispense a stream 33 of carrier drops 10 (10a, 10b . . . ), wherein the stream 33 comprises a first carrier drop 10, a second carrier drop 10a, and at least a third carrier drop 10b, and the plurality 44 of at least the liquid dispensers 1-3 is configured so that the first carrier drop 10 collides a first number of the N drops and the M drops, the second carrier drop 10b collides with a second number of the N drops and the M drops, the third carrier drop 10c collides with a third number of the N drops and the M drops, and so on, until all of the carrier drops (10a, 10b . . . ) in the stream 33 collide with at least some number of the N drops and the M drops, and all of the carrier drops 10 (10a, 10b . . . ) in the stream 33 land on the support surface 23.

Item 19 may include the device of the item 18 or some other item herein, wherein the support surface 23 is configured to collect each carrier drop 10 in the stream 33 at a unique select XY coordinate on the support surface 23, or to collect two or more carrier drops 10 (10a, 10b . . . ) at a same select XY coordinate on the support surface 23, depending on the amount of interest (N+M) of the first substance and the second substance to be prepared.

Item 20 may include the device of any one of the items 16-19 or some other item herein, wherein at least two liquid dispensers 2,3 are arranged in an array, such as a linear array or a circular array, or at least some of the liquid dispensers are arranged in at least a first array 11 and a second array 12, wherein the first array 11 and the second array 12 are configured to dispense drops towards a single stream 33 of the carrier drops 10 (10a, 10b . . . ), or the first array 11 is configured to dispense drops towards a first stream 33 of carrier drops 10 (10a, 10b . . . ), and the second array 12 is configured to dispense drops towards a second stream of carrier drops 10 (10a, 10b . . . ).

Item 21 may include the device of any one of the items 16-20 or some other item herein, wherein at least one liquid dispenser is configured to dispense drops that contain no substances of interest, and the plurality 44 of liquid dispensers is configured to collide the carrier drop 10 with at least some of the drops that contain no substances of interest, so that a lower concentration of the first substance and at least the second substance is achieved in the carrier drop 10, or no substances are contained in the carrier drop 10 when the carrier drop 10 lands, so that the carrier drop 10, which contains no substances, is used for control purposes in the sample, which is prepared.

Item 22 may include the device of any one of the items 16-21 or some other item herein, wherein at least some of liquid dispensers are configured to dispense drops that collide with the carrier drops 10 (10a, 10b . . . ) and aggregately comprise more than two substances of interest, so that the more than two substances substances are prepared at the ratio of interest, the amount of interest, and concentration of interest, at all available select XY coordinates on the support surface 23.

Item 23 may include the device of any one of the items 16-22 or some other item herein, wherein each of the liquid dispensers in the plurality 44 of liquid dispensers comprises at least a container for holding liquids to dispense, a drop generating element positioned and configured to cause at least one drop to dispense, a nozzle through which the at least one drop is ejected from, a capillary which connects the container with the nozzle so that the liquid runs from the container through the capillary and out through the nozzle for the at least one drop to be dispensed, and synchronisation means, which enable each of the liquid dispensers to operate in a synchronised manner with other liquid dispensers in the plurality 44 of liquid dispensers; and/or a single container is configured to supply liquids to two or more nozzles in the plurality 44 of liquid dispensers; and/or two or more containers are configured to supply liquids to a single nozzle in the plurality 44 of liquid dispensers.

Item 24 may include the device of any one of the items 16-23 or some other item herein, wherein the device is configured to prepare the sample on all of the available select XY coordinates on the support surface 23, so that all of the available select XY coordinates on the support surface 23 are filled with at least two substances at the ratio of interest, the amount of interest, and a concentration of interest, and/or no substances of interest.

Item 25 may include the device of any one of the items 16-24 or some other item herein, wherein the first substance, the second substance, and/or any other substance to be prepared in the sample, are substances, such as: soluble proteins, chemical additives, liquid substances used for pH control of the sample, isolated mRNA of interest, isolated gDNA of interest, isolated DNA of interest, isolated RNA of interest, regulatory plasmids, at least one DNA fragment (such as: siRNA), primers, probes, RT-qPCR Master Mix, qPCR Master mix, Master Mix, a desired concentration of APIs, solvent, anti-solvent dispersion (such as: methanol, ethanol, water, dimethyl sulfoxide (DMSO), ethyl acetate, toluene), polymers (such as: hydroxypropyl methyl cellulose (HPMC), poly vinyl alcohol (PVA), Eudragit® derivates, polyvinylpyrroldone (PVP), polyethylene glycol (PEG)), pharma polymers, surfactants (such as: polysorbate 20 Tween—Tween 20®, polysorbate 80—Tween 80®), humectants (such as: PEG, glycerol), at least one viscosity modifier (such as: PEG, glycerol), cells (such as: cells with excreted proteases, mammalian cells, E. coli cells, yeast cells, microbial cells), protein biomarkers (such as: protein metalloproteinase (MMP), various FRET-based protease substrates with or without specific inhibitors), cell encapsulation polymer used for cell encapsulation, pathogens, growth factors, metabolites, chemical of interest, a liquid used for achieving a varying concentration of any of the components listed herein, primer beads, PCR inhibitors, plasmids (such as: linearised vector encoding for bait (such as pGBKT7) protein and pray (such pGADT7) protein), DNA fragment encoding for two potential interactors (proteins) of interest, linearised vectors, fungal solution, antifungal components, at least one type of enzymes (such as HRP, ligase, polymerase), antibodies, chemical sensors, biosensors, or any other substance of interest.

Item 26 may include the device of any one of the items 16-25 or some other item herein, wherein each of the liquid dispensers in the plurality 44 of liquid dispensers is configured to dispense up to a hundred thousand (100000) drops per second, and/or is configured to dispense drops with a volume in a range between 1 picoliter to 100 nanoliters; and/or the plurality 44 of liquid dispensers comprises fifty or more, or five hundred or more, or ten thousands or more liquid dispensers.

Item 27 may include the device of any one of the items 16-26 or some other item herein, wherein the support surface 23 is: made of a bendable material or a non-bendable material, and/or is detachable or non-detachable relative to the device, and/or is a container filled with a liquid in which the carrier drops 10 (10a, 10b . . . ) land, and/or is a surface.

Item 28 may include the device of any one of the items 16-27 or some other item herein, wherein the device comprises a mechanisation 22 configured to move the support surface 23 in at least one direction relative to the plurality 44 of liquid dispensers, or to move the plurality 44 of liquid dispensers in at least one direction relative to the support surface 23, so that the carrier drops (10a, 10b . . . ) land at the select XY coordinates on the support surface 23.

Item 29 may include the device of any one of the items 16-28 or some other item herein, wherein the support surface 23 holds a standard microtiter plate or any other plastic consumable, which are used with the device, so that the sample is prepared on the select XY coordinates that correspond to select XY locations of wells in the microtiter plate or in/on the any other plastic consumable, which is positioned on the the support surface 23.

Item 30 may include the device of any one of the items 16-29 or some other item herein, wherein the device is configured to prepare the sample for the assay, such as: a high-throughput protein crystallography screening, qPCR for gene-expression studies or qPCR for SNP genotyping, ddPCR assays, a polymorph screening of engineered particles and/or compounds, any other molecular-based assays, cell encapsulation into multi-component materials studies, a single-cell line viability screening, a multiplex screening of biomarkers in cells, compound libraries screening, high-throughput forensic genotyping—Short Tandem Repeat (STR), gene expression regulation assay, ADMET (Absorption, Distribution, Metabolism, Elimination, Toxicity) assay, protein-protein interaction assays using yeast two-hybrid system, protein-DNA interaction assays using yeast one-hybrid system, microorganism activity/sensitivity assessment screening, antifungal screening on a chip, other assays wherein microorganisms are screened, biosensor validation and screening assays, rapid automated immunoassay or any other assay of interest.

Item 31 may include the device of any one of the items 16-30 or some other item herein, wherein the device comprises a processing chamber 60 wherein at least one environment regulator is installed and configured to regulate at least temperature, humidity, and CO₂ concentration in the processing chamber 60, so that the sample, which is prepared on the support surface 23 and is located in the processing chamber 60, is incubated.

Item 32 may include the device of any one of the items 16-31 or some other item herein, wherein the device comprises electric plates 21 arranged perpendicularly to a path of at least one drop dispensed from at least one liquid dispenser, wherein the electric plates 21 have holes through which the at least one drop passes, the at least one liquid dispenser is configured to provide an electric charge to the at least one drop, and the electric plates 21 are configured so that the electric plates 21 exert electric fields on the at least one drop in a synchronised manner, so that the at least one drop is manipulated with the electric fields exerted by the electric plates 21 during the time in which the at least one drop passes through the electric fields exerted by the electric plates 21.

Item 33 may include a method for preparing samples for assays, the method including dispensing and colliding drops by using a plurality 44 comprising of at least liquid dispensers 1-3, so that at least one carrier drop 10 sequentially collides with N drops that comprise a first substance and M drops that comprise at least a second substance, so that the first substance and the second substance are located at a ratio of interest (N:M) and at an amount of interest (N+M) in the carrier drop 10 during flight, and configuring the support surface 23 to so that the carrier drop 10 lands at a select XY coordinate on the support surface 23 and samples are prepared for an assay on the support surface 23 this way.

Item 34 may include a method for preparing samples for assays using the device of any one of the items 17-32 or some other item herein.

Item 35 may include a device for bioprinting multi-component 3D objects, comprising: a plurality 44 of liquid dispensers 1-3 configured to dispense and collide drops, wherein a stream 33 of n carrier drops 10 (10a, 10b . . . ) is dispensed and each of the n carrier drops 10 (10a, 10b . . . ) in the stream 33 sequentially collides with at least drops that aggregately comprise one subset of components from a set, and a support surface 23 being configured so that the n carrier drops 10 (10a, 10b . . . ) in the stream 33 land in a series of n successive XYZ coordinates that represent a multi-component 3D object, so that the multi-component 3D object is bioprinted on the support surface 23.

Item 36 may include the device of the item 35 or some other item herein, wherein at least one liquid dispenser is configured to dispense the stream 33 of the n carrier drops 10 (10a, 10b . . . ), wherein the stream 33 comprises a first carrier drop 10, a second carrier drop 10a, and at least a third carrier drop 10b, and the plurality 44 comprising of at least the liquid dispensers 1-3 are configured so that the first carrier drop 10 collides with at least drops that aggregately comprise a first subset of components, the second carrier drop 10a collides with at least drops that aggregately comprise a second subset of components, the third carrier drop 10b collides with at least drops that aggregately comprise a third subset components from, and so on, until all of the n carrier drops 10 (10a, 10b . . . ) in the stream 33 collide with at least some drops that aggregately comprise at least some appropriate components from the set, and all of the n carrier drops 10 (10a, 10b . . . ) in the stream 33 land on the support surface 23.

Item 37 may include the device of the item 36 or some other item herein, wherein the n carrier drops 10 (10a, 10b . . . ) include a cell-carrying bio-ink, such as a biopolymer gel, at the time of dispensing, or the n carrier drops 10 (10a, 10b . . . ) include at least one type of cells suspended in the cell-carrying bio-ink, such as the biopolymer gel.

Item 38 may include the device of any one of the items 35-37 or some other item herein, wherein the set comprises of components, such as: biological materials, biomaterials, metabolites, growth media, growth serums, growth factors, or other components of the bioprinted multi-component 3D object.

Item 39 may include the device of any one of the items 35-38 or some other item herein, wherein at least two liquid dispensers 2,3 are arranged in an array, such as a linear array or any other suitable type of array, and/or the each of the n carrier drops 10 (10a, 10b . . . ) has a trajectory that passes by the at least two liquid dispensers 2,3 configured to dispense drops towards the trajectory of the carrier drop 10 in a synchronised manner, so that the carrier drop 10 sequentially collides with the drops that aggregately comprise components from the subset and are dispensed by some of the at least two liquid dispensers 2,3 in the array.

Item 40 may include the device of any one of the items 35-39 or some other item herein, wherein at least some of the liquid dispensers are arranged in at least a first array 11 and a second array 12, wherein the first array 11 and the second array 12 are configured to dispense drops towards a single stream 33 of the carrier drops 10 (10a, 10b . . . ), or the first array 11 is configured to dispense drops towards a first stream 33 of carrier drops 10 (10a, 10b . . . ), and the second array 12 is configured to dispense drops towards a second stream of carrier drops 10 (10a, 10b . . . ).

Item 41 may include the device of any one of the items 35-40 or some other item herein, wherein the device comprises a mechanisation 22 configured to: move the support surface 23 in at least one direction relative to the plurality 44 of liquid dispensers, and/or move the plurality 44 of liquid dispensers in at least one direction relative to the support surface 23, so that the n carrier drops 10 (10a, 10b . . . ) in the stream 33 land in the series of n successive XYZ coordinates on the support surface 23.

Item 42 may include the device of any one of the items 35-41 or some other item herein, wherein the device comprises a mixing chamber 50 wherein the plurality 44 of liquid dispensers, the support surface 23, and at least one environment regulator are positioned, and the environment regulator is configured to regulate at least: temperature, humidity, and CO₂ concentration in the mixing chamber 50.

Item 43 may include the device of any one of the items 35-42 or some other item herein, wherein the device comprises a processing chamber 60 wherein at least one processing element 401 is installed and configured to regulate the temperature, humidity, CO₂ concentration and light in the processing chamber 60, so that incubation of the multi-component 3D object, which is located in the processing chamber 60, occurs.

Item 44 may include the device of any one of the items 35-43 or some other item herein, wherein each of the liquid dispensers in the plurality 44 of liquid dispensers comprises at least a container for holding liquids to dispense, a drop generating element positioned and configured to cause at least one drop to dispense, a nozzle through which the at least one drop is ejected from, a capillary which connects the container with the nozzle so that the liquid runs from the container through the capillary and out through the nozzle for the at least one drop to be dispensed, and synchronisation means, which enable each of the liquid dispensers to operate in a synchronised manner with other liquid dispensers in the plurality 44 of liquid dispensers; and/or a single container is configured to supply liquids to two or more nozzles in the plurality 44 of liquid dispensers; and/or two or more containers are configured to supply liquids to a single nozzle in the plurality 44 of liquid dispensers.

Item 45 may include the device of any one of the items 35-44 or some other item herein, wherein the device is configured to bioprint the multi-component3D object such as: an organ, an organ on a chip, a tissue, a scaffold, or any other bioprintable multi-component 3D object.

Item 46 may include the device of any one of the items 35-45 or some other item herein, wherein the device comprises electric plates 21 arranged perpendicularly to a path of at least one drop dispensed from at least one liquid dispenser, wherein the electric plates 21 have holes through which the at least one drop passes, the at least one liquid dispenser is configured to provide an electric charge to the at least one drop, and the electric plates 21 are configured so that the electric plates 21 exert electric fields on the at least one drop in a synchronised manner, so that the at least one drop is manipulated with the electric fields exerted by the electric plates 21 during the time in which the at least one drop passes through the electric fields exerted by the electric plates 21.

Item 47 may include a method for bioprinting multi-component 3D objects, the method including dispensing and colliding drops by using a plurality 44 of at least liquid dispensers 1-3, so that a stream 33 of n carrier drops 10 (10a, 10b . . . ) is dispensed and each of the n carrier drops 10 (10a, 10b . . . ) collides and merges with at least drops that aggregately comprise one subset of components from a set during flight, and configuring a support surface 23 so that the n carrier drops (10a, 10b . . . ) land in a series of n successive XYZ coordinates, which represent a multi-component 3D object, so that the multi-component 3D object is bioprinted on the support surface 23.

Item 48 may include the method of the item 47 or some other item herein, wherein the method includes converting, by using a central control 1000 unit, a 3D model of the multi-component 3D object into the series of n successive XYZ coordinates, and assigning, by using the control unit, one subset of components from the set to each XYZ coordinate in the series of n successive XYZ coordinates, so that the multi-component 3D object is represented by the series of n successive XYZ coordinates wherein each of the XYZ coordinates has the subset of components assigned.

Item 49 may include a method using the device of any one of the items 36-46 or some other item herein.

Item 50 may include a device for 3D printing multi-material 3D objects comprising a plurality 44 of liquid dispensers 1-3 configured to dispense and collide drops, wherein a stream 33 of n carrier drops 10 (10a, 10b . . . ) is dispensed and each of the n carrier drops 10 (10a, 10b . . . ) in the stream 33 sequentially collides with at least drops that aggregately comprise one subset of materials from a set, and a support surface 23 being configured so that the n carrier drops 10 (10a, 10b . . . ) in the stream 33 land in a series of n successive XYZ coordinates that represent a multi-material 3D object, so that the multi-material 3D object is 3D printed on the support surface 23.

Item 51 may include the device of the item 50 or some other item herein, wherein at least one liquid dispenser is configured to dispense the stream 33 of the n carrier drops 10 (10a, 10b . . . ), wherein the stream 33 comprises a first carrier drop 10, a second carrier drop 10a, and at least a third carrier drop 10b, and the plurality 44 comprising of at least the liquid dispensers 1-3 is configured so that the first carrier drop 10 collides with at least drops that aggregately comprise a first subset of materials, the second carrier drop 10a collides with at least drops that aggregately comprise a second subset of materials, the third carrier drop 10b collides with at least drops that aggregately comprise a third subset materials from, and so on, until all of the n carrier drops 10 (10a, 10b . . . ) in the stream 33 collide with at least some drops that aggregately comprise at least some appropriate materials from the set, and all of the n carrier drops 10 (10a, 10b . . . ) in the stream 33 land in the series of n successive XYZ coordinates on the support surface 23.

Item 52 may include the device of the item 50 or some other item herein, wherein the n carrier drops 10 (10a, 10b . . . ) include a binder material, such as a polymer, a photopolymer, or a water based binder, at the time of dispensing, or the n carrier drops 10 (10a, 10b . . . ) include at least one type of material particles dispensed in the binder material.

Item 53 may include the device of any one of the items 50-52 or some other item herein, wherein the set comprises of materials, such as: metal powders or granules dispensed in liquids, ceramic powders or granules dispensed in liquids, polymers, binders, or other materials of the 3D printed multi-material 3D object.

Item 54 may include the device of any one of the items 50-53 or some other item herein, wherein at least two liquid dispensers 2,3 are arranged in an array, such as a linear array or any other suitable type of array, and/or the each of the n carrier drops 10 (10a, 10b . . . ) has a trajectory that passes by the at least two liquid dispensers 2,3 configured to dispense drops towards the trajectory of the carrier drop 10 in a synchronised manner, so that the carrier drop 10 sequentially collides with the drops that aggregately comprise materials from the subset and are dispensed by some of the at least two liquid dispensers 2,3 in the array.

Item 55 may include the device of any one of the items 50-54 or some other item herein, wherein at least some of the liquid dispensers are arranged in at least a first array 11 and a second array 12, wherein the first array 11 and the second array 12 are configured to dispense drops towards a single stream 33 of the carrier drops 10 (10a, 10b . . . ), or the first array 11 is configured to dispense drops towards a first stream 33 of carrier drops 10 (10a, 10b . . . ), and the second array 12 is configured to dispense drops towards a second stream of carrier drops 10 (10a, 10b . . . ).

Item 56 may include the device of any one of the items 50-55 or some other item herein, wherein the device comprises a mechanisation 22 configured to: move the support surface 23 in at least one direction relative to the plurality 44 of liquid dispensers, and/or move the plurality 44 of liquid dispensers in at least one direction relative to the support surface 23, so that the n carrier drops 10 (10a, 10b . . . ) in the stream 33 land in the series of n successive XYZ coordinates on the support surface 23.

Item 57 may include the device of any one of the items 50-56 or some other item herein, wherein the device comprises a mixing chamber 50 wherein the plurality 44 of liquid dispensers, the support surface 23, and at least one environment regulator are positioned, and the environment regulator is configured to regulate at least a temperature and a humidity in the mixing chamber 50 over time.

Item 58 may include the device of any one of the items 50-57 or some other item herein, wherein the plurality 44 of liquid dispensers comprise a temperature regulation system configured to regulate a temperature of the materials each liquid dispenser in the plurality 44 of liquid dispensers before drops are dispensed, so that the drops are dispensed at a correct temperature.

Item 59 may include the device of any one of the items 50-58 or some other item herein, wherein each of the liquid dispensers in the plurality 44 of liquid dispensers comprises a container for holding liquids to dispense, a drop generating element positioned and configured to cause at least one drop to dispense, a nozzle through which the at least one drop is ejected from, a capillary which connects the container with the nozzle so that the liquid runs from the container through the capillary and out through the nozzle for the at least one drop to be dispensed, and synchronisation means, which enable each of the liquid dispensers to operate in a synchronised manner with other liquid dispensers in the plurality 44 of liquid dispensers; and/or a single container is configured to supply liquids to two or more nozzles in the plurality 44 of liquid dispensers; and/or two or more containers are configured to supply liquids to a single nozzle in the plurality 44 of liquid dispensers.

Item 60 may include the device of the item 59 or some other item herein, wherein at least two liquid dispensers 2,3 are configured to dispense drops that contain a same material from the set, and the plurality 44 of liquid dispensers is configured so that each of the n carrier drops 10 (10a, 10b . . . ) collides with a different number of drops, which comprise the same material, a desired amount and density of the same material is contained in the each of the n carrier drops 10 (10a, 10b . . . ) and the desired amount and density varies across the multi-material 3D object, which is 3D printed, so that a functionally graded multi-material 3D object is 3D printed, wherein a physical property of the functionally graded multi-material 3D object, such as density, varies in at least one direction in the functionally graded multi-material 3D object.

Item 61 may include the device of any one of the items 50-60 or some other item herein, wherein the device comprises electric plates 21 arranged perpendicularly to a path of at least one drop dispensed from at least one liquid dispenser, wherein the electric plates 21 have holes through which the at least one drop passes, the at least one liquid dispenser is configured to provide an electric charge to the at least one drop, and the electric plates 21 are configured so that the electric plates 21 exert electric fields on the at least one drop in a synchronised manner, so that the at least one drop is manipulated with the electric fields exerted by the electric plates 21 during the time in which the at least one drop passes through the electric fields exerted by the electric plates 21.

Item 62 may include a method for 3D printing multi-material 3D objects, the method comprising dispensing and colliding drops using a plurality 44 comprising of at least liquid dispensers 1-3, so that a stream 33 of n carrier drops 10 (10a, 10b . . . ) is dispensed and each of the n carrier drops 10 (10a, 10b . . . ) collides and merges with at least drops that aggregately comprise one subset of materials from a set during flight, and configuring a support surface 23 so that the n carrier drops 10 (10a, 10b . . . ) land in a series of n successive XYZ coordinates, which represent a multi-material 3D object, so that the multi-material 3D object is 3D printed on the support surface 23.

Item 63 may include the method of the item 62 or some other item herein, includes converting, by using a central control 1000 unit, a 3D model of the multi-material 3D object into the series of n successive XYZ coordinates, and assigning, by using the control unit, one subset of components from the set to each of XYZ coordinates in the series of n successive XYZ coordinates coordinates, so that the multi-material 3D object is represented by the series of n successive XYZ coordinates coordinates wherein each of the XYZ coordinates has the subset of components assigned.

Item 64 may include a method for 3D printing multi-material 3D objects using the device of any one of the items 51-61 or some other item herein.

Item 65 may include a device for manufacturing multi-substance tablets for oral delivery, the device comprising a plurality 44 of liquid dispensers 1-3 configured to dispense and collide drops, wherein a stream 33 of n carrier drops 10 (10a, 10b . . . ) is dispensed and each of the n carrier drops 10 (10a, 10b . . . ) in the stream 33 sequentially collides with at least drops that aggregately comprise one subset of active pharmaceutical ingredients (hereinafter: APIs) and/or excipients from a set, and a support surface 23 being configured so that the n carrier drops 10 (10a, 10b . . . ) in the stream 33 land in a series of n successive XYZ coordinates that represent a plurality of multi-substance tablets, so that the plurality of multi-substance tablets is manufactured on the support surface 23.

Item 66 may include the device of the item 65 or some other item herein, wherein at least one liquid dispenser is configured to dispense the stream 33 of the n carrier drops 10 (10a, 10b . . . ), wherein the stream 33 comprises a first carrier drop 10, a second carrier drop 10a, and at least a third carrier drop 10b, and the plurality 44 comprising of at least the liquid dispensers 1-3 are configured so that the first carrier drop 10 collides with at least drops that aggregately comprise a first subset of APIs and/or excipients, the second carrier drop 10a collides with at least drops that aggregately comprise a second subset of APIs and/or excipients, the third carrier drop 10b collides with at least drops that aggregately comprise a third subset of APIs and/or excipients from, and so on, until all of the n carrier drops 10 (10a, 10b . . . ) in the stream 33 collide with at least some drops that aggregately comprise at least some appropriate APIs and/or excipients from the set, and all of the n carrier drops 10 (10a, 10b . . . ) in the stream 33 land in the series of n successive XYZ coordinates on the support surface 23.

Item 67 may include the device of the item 65 or some other item herein, wherein the n carrier drops 10 (10a, 10b . . . ) include a liquid ink carrier, such as: water, ethanol, propanol, or any other appropriate liquid ink carrier, or the n carrier drops 10 (10a, 10b . . . ) include at least one API and the liquid ink carrier, or the n carrier drops 10 (10a, 10b . . . ) include at least the liquid ink carrier and an excipient dispersion, so that rheological properties of the liquid ink carrier are customised.

Item 68 may include the device of any one of the items 65-67 or some other item herein, wherein the set comprises of APIs and/or excipients, such as: APIs, polymers (such as but not limited to: hydroxypropyl methyl cellulose (HPMC), poly vinyl alcohol (PVA), Eudragit @derivates, polyvinylpyrroldone (PVP), polyethylene glycol (PEG)), surfactants (such as, but not limited to polysorbate 20—Tween 20®, polysorbate 80—Tween 80® or other), liquid ink carriers (such as: water, ethanol, propanol, or any combination of the liquid ink carriers listed herein), humectants (such as but not limited to: PEG, glycerol, or other), viscosity modifiers (such as but not limited to: glycerol, PEG, or other), solid excipient powder or granulate mixture, which may consist of various diluents, binding agents, disintegrants, lubricants, tablet coatings and films, colouring agents or other ingredients of the plurality of multi-substance tablets.

Item 69 may include the device of any one of the items 65-68 or some other item herein, wherein at least two liquid dispensers 2,3 are arranged in an array, such as a linear array or any other suitable type of array, and/or the each of the n carrier drops 10 (10a, 10b . . . ) has a trajectory that passes by the at least two liquid dispensers 2,3 configured to dispense drops towards the trajectory of the carrier drop 10 in a synchronised manner, so that the carrier drop 10 sequentially collides with the drops that aggregately comprise APIs and/or excipients from the subset and are dispensed by some of the at least two liquid dispensers 2,3 in the array.

Item 70 may include the device of any one of the items 65-69 or some other item herein, wherein the plurality 44 of liquid dispensers is configured so that some of the n carrier drops 10 (10a, 10b . . . ) in the stream 33 collide with the drops that contain binder agents, and some other of the n carrier drops 10 (10a, 10b . . . ) collide with the drops that contain solid excipients powders or granule mixtures suspended in highly volatile liquids, which evaporate before the carrier drops 10 (10a, 10b . . . ) land, so that a high porosity of the plurality of multi-substance tablets is achieved, and the high porosity results in high disintegration.

Item 71 may include the device of any one of the items 65-70 or some other item herein, wherein at least some liquid dispensers, which dispense drops that collide with any of the n carrier drops 10 (10a, 10b . . . ), are configured to dispense drops that contain agents, which cause a sustained release of the APIs or a modified release of the APIs in the plurality of multi-substance tablets, so that a sustained release of the plurality of multi-substance tablets or a modified release of the plurality multi-substance tablets is achieved.

Item 72 may include the device of any one of the items 65-71 or some other item herein, wherein the device comprises a mechanisation 22 configured to: move the support surface 23 in at least one direction relative to the plurality 44 of liquid dispensers, and/or move the plurality 44 of liquid dispensers in at least one direction relative to the support surface 23, so that the n carrier drops 10 (10a, 10b . . . ) in the stream 33 land in the series of n successive XYZ coordinates on the support surface 23.

Item 73 may include the device of any one of the items 65-72 or some other item herein, wherein the device comprises a mixing chamber 50 wherein the plurality 44 of liquid dispensers, the support surface 23, and at least one environment regulator are positioned, and the environment regulator is configured to regulate at least a temperature and a humidity in the mixing chamber 50 over time.

Item 74 may include the device of any one of the items 65-73 or some other item herein, wherein at least some of the liquid dispensers are arranged in at least a first array 11 and a second array 12, wherein the first array 11 and the second array 12 are configured to dispense drops towards a single stream 33 of the carrier drops 10 (10a, 10b . . . ), or the first array 11 is configured to dispense drops towards a first stream 33 of carrier drops 10 (10a, 10b . . . ), and the second array 12 is configured to dispense drops towards a second stream of carrier drops 10 (10a, 10b . . . ).

Item 75 may include the device of any one of the items 65-74 or some other item herein, wherein each of the liquid dispensers in the plurality 44 of liquid dispensers comprises a container for holding liquids to dispense, a drop generating element positioned and configured to cause at least one drop to dispense, a nozzle through which the at least one drop is ejected from, a capillary which connects the container with the nozzle so that the liquid runs from the container through the capillary and out through the nozzle for the at least one drop to be dispensed, and synchronisation means, which enable each of the liquid dispensers to operate in a synchronised manner with other liquid dispensers in the plurality 44 of liquid dispensers; and/or a single container is configured to supply liquids to two or more nozzles in the plurality 44 of liquid dispensers; and/or two or more containers are configured to supply liquids to a single nozzle in the plurality 44 of liquid dispensers.

Item 76 may include the device of any one of the items 65-75 or some other item herein, wherein the device comprises electric plates 21 arranged perpendicularly to a path of at least one drop dispensed from at least one liquid dispenser, wherein the electric plates 21 have holes through which the at least one drop passes, the at least one liquid dispenser is configured to provide an electric charge to the at least one drop, and the electric plates 21 are configured so that the electric plates 21 exert electric fields on the at least one drop in a synchronised manner, so that the at least one drop is manipulated with the electric fields exerted by the electric plates 21 during the time in which the at least one drop passes through the electric fields exerted by the electric plates 21.

Item 77 may include a method for manufacturing multi-substance tablets for oral delivery, the method comprising of dispensing and colliding drops using a plurality 44 comprising of at least liquid dispensers 1-3, so that a stream 33 of n carrier drops 10 (10a, 10b . . . ) is dispensed and each of the n carrier drops 10 (10a, 10b . . . ) collides and merges with at least drops that aggregately comprise one subset of active pharmaceutical ingredients (hereinafter: APIs) and/or excipients from a set during flight, and configuring a support surface 23 so that the n carrier drops 10 (10a, 10b . . . ) land in the series of n successive XYZ coordinates, which represent a plurality of multi-substance tablets, so that the plurality of multi-substance tablets is manufactured on the support surface 23.

Item 78 may include the method of the item 77 or some other item herein, wherein the method includes converting, by using a central control 1000 unit, a 3D model of the plurality of multi-substance tablets into the series of n successive XYZ coordinates, and assigning, by using the control unit, one subset of APIs and/or excipients from the set to each of XYZ coordinates in the series of n successive XYZ coordinates, so that the plurality of multi-substance tablets is represented by the series of n successive XYZ coordinates wherein each of the XYZ coordinates has the subset of components assigned.

Item 79 may include a method for manufacturing multi-substance tablets using the device of any one of the items 66-76 or some other item herein.

Item 80 may include a device for manufacturing micro and/or nano sized therapeutical particles for parerental, respiratory or nasal delivery, the device comprising a plurality 44 of liquid dispensers 1-3 configured to dispense and collide drops, wherein a stream 33 of n carrier drops 10 (10a, 10b . . . ) is dispensed and each of the n carrier drops 10 (10a, 10b . . . ) in the stream 33 sequentially collides with at least drops that aggregately comprise one subset of active pharmaceutical ingredients (hereinafter: APIs) and/or excipients from a set;
and a support surface 23 being configured so that the n carrier drops 10 (10a, 10b . . . ) in the stream 33 land in a series of select XY coordinates, so that the micro and/or nano sized therapeutical particles are manufactured in the select XY coordinates on the support surface 23.

Item 81 may include the device of the item 80 or some other item herein, wherein at least one liquid dispenser is configured to dispense the stream 33 of the n carrier drops 10 (10a, 10b . . . ), wherein the stream 33 comprises a first carrier drop 10, a second carrier drop 10a, and at least a third carrier drop 10b, and the plurality 44 comprising of at least the liquid dispensers 1-3 are configured so that the first carrier drop 10 collides with at least drops that aggregately comprise a first subset of APIs and/or excipients, the second carrier drop 10a collides with at least drops that aggregately comprise a second subset of APIs and/or excipients, the third carrier drop 10b collides with at least drops that aggregately comprise a third subset of APIs and/or excipients from, and so on, until all of the n carrier drops 10 (10a, 10b . . . ) in the stream 33 collide with at least some drops that aggregately comprise at least some appropriate APIs and/or excipients from the set, and all of the n carrier drops 10 (10a, 10b . . . ) in the stream 33 land in the series of select XY coordinates on the support surface 23.

Item 82 may include the device of the item 80 or 81 or some other item herein, wherein the n carrier drops 10 (10a, 10b . . . ) include an excipient dispersion that is capable of carrying substances of the micro and/or nano sized therapeutical particles, or the n carrier drops 10 (10a, 10b . . . ) include the excipient dispersion and at least one API, or the n carrier drops 10 (10a, 10b . . . ) comprise anti-solvent liquids, such as: water, tert-butanol, ethanol, so that substances contained in drops that collide with the carrier drops 10 (10a, 10b . . . ), do not solve in the carrier drops 10 (10a, 10b . . . ) comprising the anti-solvent liquids.

Item 83 may include the device of any one of the items 80-82 or some other item herein, wherein the set comprises of APIs and/or excipients, such as: APIs (therapeutic peptides or biologically-active small molecules), biodegradable carrier polymer for sustained-release (such as: poly lactide (PLA), poly (lactic-co-glycolic) acid (PLGA)), liquid ink carriers (such as: water, dimethyl sulfoxide (DMSO), ethyl acetate), anti-solvent liquids (such as: water, tert-butanol, ethanol) or other substances, which constitute the micro and/or nano sized therapeutical particles.

Item 84 may include the device of any one of the items 80-83 or some other item herein, wherein at least two liquid dispensers 2,3 are arranged in an array, such as a linear array or any other suitable type of array, and/or the each of the n carrier drops 10 (10a, 10b . . . ) has a trajectory that passes by the at least two liquid dispensers 2,3 configured to dispense drops towards the trajectory of the carrier drop 10 in a synchronised manner, so that the carrier drop 10 sequentially collides with the drops that aggregately comprise APIs and/or excipients from the subset and are dispensed by some of the at least two liquid dispensers 2,3 in the array.

Item 85 may include the device of any one of the items 80-84 or some other item herein, wherein the device comprises a freeze drying element mounted on the support surface 23, wherein the freeze-drying element is configured to freeze dry the carrier drops 10 (10a, 10b . . . ) immediately after the carrier drops 10 (10a, 10b . . . ) land on the support surface 23, so that spherically shaped micro and/or nano sized therapeutical particles are produced.

Item 86 may include the device of any one of the items 80-85 or some other item herein, wherein the select XY coordinates correlate with internal part of vials or cartridges (for inhalation devices), which are positioned on the support surface 23, and a Z coordinate relates to an amount of micro and/or nano sized therapeutical particles to be deposited in the vials or cartridges (for inhalation devices), which are positioned at the select XY coordinates on the support surface 23.

Item 87 may include the device of any one of the items 80-86 or some other item herein, wherein the device comprises a mechanisation 22 configured to: move the support surface 23 in at least one direction relative to the plurality 44 of liquid dispensers, and/or move the plurality 44 of liquid dispensers in at least one direction relative to the support surface 23, so that the n carrier drops 10 (10a, 10b . . . ) in the stream 33 land in at least the select XY coordinates on the support surface 23.

Item 88 may include the device of any one of the items 80-87 or some other item herein, wherein the device comprises a mixing chamber 50 wherein the plurality 44 of liquid dispensers, the support surface 23, and at least one environment regulator are positioned, and the environment regulator is configured to regulate at least a temperature and a humidity in the mixing chamber 50 over time.

Item 89 may include the device of the item 88 or some other item herein, wherein the environment regulator is configured to provide the temperature and the humidity in the mixing chamber 50 so that the carrier drops 10 (10a, 10b . . . ) evaporate or spray dry before landing in at least the select XY coordinates, so that the spherically shaped nano and/or micro sized therapeutical particles are produced.

Item 90 may include the device of any one of the items 80-89 or some other item herein, wherein at least some of the liquid dispensers are arranged in at least a first array 11 and a second array 12, wherein the first array 11 and the second array 12 are configured to dispense drops towards a single stream 33 of the carrier drops 10 (10a, 10b . . . ), or the first array 11 is configured to dispense drops towards a first stream 33 of carrier drops 10 (10a, 10b . . . ), and the second array 12 is configured to dispense drops towards a second stream of carrier drops 10 (10a, 10b . . . ).

Item 91 may include the device of any one of the items 80-90 or some other item herein, wherein each of the liquid dispensers in the plurality 44 of liquid dispensers comprises a container for holding liquids to dispense, a drop generating element positioned and configured to cause at least one drop to dispense, a nozzle through which the at least one drop is ejected from, a capillary which connects the container with the nozzle so that the liquid runs from the container through the capillary and out through the nozzle for the at least one drop to be dispensed, and synchronisation means, which enable each of the liquid dispensers to operate in a synchronised manner with other liquid dispensers in the plurality 44 of liquid dispensers; and/or a single container is configured to supply liquids to two or more nozzles in the plurality 44 of liquid dispensers; and/or two or more containers are configured to supply liquids to a single nozzle in the plurality 44 of liquid dispensers.

Item 92 may include the device of any one of the items 80-91 or some other item herein, wherein the device comprises electric plates 21 arranged perpendicularly to a path of at least one drop dispensed from at least one liquid dispenser, wherein the electric plates 21 have holes through which the at least one drop passes, the at least one liquid dispenser is configured to provide an electric charge to the at least one drop, and the electric plates 21 are configured so that the electric plates 21 exert electric fields on the at least one drop in a synchronised manner, so that the at least one drop is manipulated with the electric fields exerted by the electric plates 21 during the time in which the at least one drop passes through the electric fields exerted by the electric plates 21.

Item 93 may include the device of the item 82 or some other item herein, wherein the device comprises a processing chamber 60 wherein at least one processing element 401 is installed, and the at least one processing element 401 is configured to remove the anti-solvent liquids from the carrier drops 10 (10a, 10b . . . ), which are located in the processing chamber 60 and comprise the anti-solvent liquids.

Item 91 may include a method for manufacturing micro and/or nano sized therapeutical particles for parerental, respiratory or nasal delivery, the method comprising dispensing and colliding drops using a plurality 44 of liquid dispensers comprising of at least liquid dispensers 1-3, so that a stream 33 of n carrier drops 10 (10a, 10b . . . ) is dispensed and each of the n carrier drops 10 (10a, 10b . . . ) collides and merges with at least drops that aggregately comprise one subset of APIs and/or excipients from the set during flight, and configuring the support surface 23 so that the n carrier drops 10 (10a, 10b . . . ) land in at least a series of select XY coordinates, so that micro and/or nano sized therapeutical particles are manufactured on the support surface 23.

Item 95 may include a method for manufacturing micro and/or nano sized therapeutical particles for parerental, respiratory or nasal delivery using the device of any one of the items 80-93 or some other item herein.

Item 96 may include a device for applying active pharmaceutical ingredients (hereinafter: APIs) and/or excipients on pre-manufactured placebo pharmaceuticals, the device comprising: a plurality 44 of liquid dispensers 1-3 being configured to dispense drops, wherein a stream 33 of carrier drops 10 (10a, 10b . . . ) is dispensed, and each of the carrier drops 10 (10a, 10b . . . ) in the stream 33 sequentially collides with a pre-determined number of the drops that aggregately comprise one subset of APIs and/or excipients selected from a set of APIs and/or excipients, so that a pre-determined amount and combination of APIs and/or excipients is contained in each of the carrier drops 10 (10a, 10b . . . ) in the stream 33 during flight; and a support surface 23 being configured so that the each of the carrier drops 10 (10a, 10b . . . ) in the stream 33 lands on one of select XY coordinates on a plurality of pre-manufactured placebos positioned on the support surface 23.

Item 97 may include the device of the item 96 or some other item herein, wherein at least one liquid dispenser 1 is configured to dispense the stream 33 of carrier drops 10 (10a, 10b . . . ), and/or the stream 33 of carrier drop 10 has a trajectory that passes by at least two liquid dispensers 2,3 configured to dispense drops towards the trajectory of the stream 33 of carriers drop in a synchronised manner, so that each of the carrier drop 10 in the stream 33 sequentially collides with at least drops that aggregately comprise one pre-determined amount and combination of APIs and/or excipients from the set and are dispensed by some of the at least two liquid dispensers 2,3.

Item 98 may include the device of the item 96 or 97 or some other item herein, wherein the stream 33 comprises a first carrier drop 10, a second carrier drop 10a, and at least a third carrier drop 10b, and the plurality 44 comprising of at least the liquid dispensers 1-3 are configured so that the first carrier drop 10 collides with drops that aggregately comprise a first pre-determined amount and combination of APIs and/or excipients, the second carrier drop 10a collides with drops that aggregately comprise a second pre-determined amount and combination of APIs and/or excipients, the third carrier drop 10b collides with drops that aggregately comprise a third pre-determined amount and combination of APIs and/or excipients, and so on, until all of the carrier drops 10 (10a, 10b . . . ) in the stream 33 collide with at least some drops that aggregately comprise any desired pre-determined amount and combination of APIs and/or excipients from the set, all of the carrier drops 10 (10a, 10b . . . ) in the stream 33 land on the plurality of pre-manufactured placebos positioned on the support surface 23, and a variable dosing of APIs and/or excipients is achieved across the plurality of pre-manufactured placebos.

Item 99 may include the device of any one of the items 96-98 or some other item herein, wherein the at least two liquid dispensers 2,3 are arranged in an array, such as a linear array or a circular array; or at least some of the liquid dispensers are arranged in at least a first array 11 and a second array 12, wherein the first array 11 and the second array 12 are configured to dispense drops towards a single stream 33 of the carrier drops 10 (10a, 10b . . . ), or the first array 11 is configured to dispense drops towards a first stream 33 of carrier drops 10 (10a, 10b . . . ), and the second array 12 is configured to dispense drops towards a second stream of carrier drops 10 (10a, 10b . . . ).

Item 100 may include the device of any one of the items 96-99 or some other item herein, wherein the carrier drops include high loaded APIs at the time of dispensing.

Item 101 may include the device of any one of the items 96-100 or some other item herein, wherein the support surface 23 is positioned so that two or more carrier drops 10 (10a, 10b . . . ) from the stream 33 land at a single select XY coordinate whereat one pre-manufactured placebo is located, wherein the pre-determined amount of APIs and/or excipients is contained in the two or more carrier drops 10 (10a, 10b . . . ).

Item 102 may include the device of any one of the items 96-101 or some other item herein, wherein the plurality 44 of liquid dispensers is configured so that each API from the set is suspended in a liquid in a single container that supplies the liquid to at least two liquid dispensers 2,3 in the plurality 44 of liquid dispensers, and each excipient from the set is suspended in another liquid in another container, which supplies the another liquid to another at least two liquid dispensers 2,3 in the plurality 44 of liquid dispensers.

Item 102 may include the device of any one of the items 96-102 or some other item herein, wherein each of the liquid dispensers in the plurality 44 of liquid dispensers comprises a container for holding liquids that include one API and/or excipient from the set to dispense, a drop generating element positioned and configured to cause at least one drop to dispense, a nozzle through which the at least one drop is ejected from, a capillary which connects the container with the nozzle so that the liquid runs from the container through the capillary and out through the nozzle for the at least one drop to be dispensed, and synchronisation means, which enable each of the liquid dispensers to operate in a synchronised manner with other liquid dispensers in the plurality 44 of liquid dispensers; and/or a single container is configured to supply liquids to two or more nozzles in the plurality 44 of liquid dispensers; and/or two or more containers are configured to supply liquids to a single nozzle in the plurality 44 of liquid dispensers.

Item 104 may include the device of any one of the items 96-103 or some other item herein, wherein the device comprises a mechanisation 22 configured to move the support surface 23 in at least one direction relative to the plurality 44 of liquid dispensers, and/or move the plurality 44 of liquid dispensers in at least one direction relative to the support surface 23.

Item 105 may include the device of any one of the items 96-104 or some other item herein, wherein the device comprises a mixing chamber 50, which is a partially or a fully closed compartment of the device wherein the plurality 44 of liquid dispensers and at least one support surface 23 are positioned, and the mixing chamber 50 is the fully closed compartment during the time in which the drops are in-flight.

Item 106 may include the device of any one of the items 96-105 or some other item herein, wherein the set comprises APIs and/or excipients, such as: APIs, polymers (such as: hydroxypropyl methyl cellulose (HPMC), poly vinyl alcohol (PVA), Eudragit® derivates, polyvinylpyrroldone (PVP), polyethylene glycol (PEG)), surfactants (such as: Polysorbate 20—Tween 20® or other), liquid ink carriers (such as: water, ethanol, propanol, or any combination of liquid ink carriers), humectants (such as: PEG, glycerol, or other), viscosity modifiers (such as but not limited to: glycerol, PEG, or other) or other ingredients, which are applied on to the pre-manufactured placebo therapeutics.

Item 107 may include the device of any one of the items 96-106 or some other item herein, wherein the plurality of pre-manufactured placebos positioned on the support surface 23, are: oral tablets, capsules, orodispensible films, films in capsules, mucoadhesive films or other pre-manufactured placebos.

Item 108 may include the device of any one of the items 96-107 or some other item herein, wherein the device comprises electric plates 21 arranged perpendicularly to a path of at least one drop dispensed from at least one liquid dispenser, wherein the electric plates 21 have holes through which the at least one drop passes, the at least one liquid dispenser is configured to provide an electric charge to the at least one drop, and the electric plates 21 are configured so that the electric plates 21 exert electric fields on the at least one drop in a synchronised manner, so that the at least one drop is manipulated with the electric fields exerted by the electric plates 21 during the time in which the at least one drop passes through the electric fields exerted by the electric plates 21

Item 109 may include a method for applying active pharmaceutical ingredients (hereinafter: APIs) and/or excipients on pre-manufactured placebos, the method including dispensing and colliding drops, by using a plurality 44 of at least liquid dispensers 1-3, so that a stream 33 of carrier drops 10 is dispensed and each of the carrier drops 10 in the stream 33 collides with drops that aggregately comprise one subset of APIs and/or excipients from a set, so that a pre-determined amount and combination of APIs and/or excipients is located in each of the carrier drops 10 during flight, and configuring a support surface 23, so that the each of the carrier drops 10 lands on one of select XY coordinates on a plurality of pre-manufactured placebos positioned on the support surface 23.

Item 110 may include a method for applying APIs and/or excipients on pre-manufactured placebos using the device in any one of the items 97-108 or some other item herein.

Item 111 may include a device for applying active pharmaceutical ingredients (hereinafter: APIs) and/or excipients on medical devices, the device comprising: a plurality 44 of liquid dispensers 1-3 being configured to dispense drops, wherein a stream 33 of carrier drops 10 (10a, 10b . . . ) is dispensed, and each of the carrier drops 10 (10a, 10b . . . ) in the stream 33 sequentially collides with a pre-determined number of the drops that aggregately comprise one subset of APIs and/or excipients selected from a set, so that a pre-determined amount and combination of APIs and/or excipients is located in each of the carrier drops 10 (10a, 10b . . . ) in the stream 33 during flight; and a support surface 23 being configured so that the each of the carrier drops 10 (10a, 10b . . . ) in the stream 33 lands on one of select XYZ coordinates whereat a medical device is positioned on the support surface 23.

Item 112 may include the device of the item 111 or some other item herein, wherein at least one liquid dispenser 1 is configured to dispense the stream 33 of carrier drops 10 (10a, 10b . . . ), and/or the stream 33 of carrier drop 10 has a trajectory that passes by at least two liquid dispensers 2,3 configured to dispense drops towards the trajectory of the stream 33 of carriers drop in a synchronised manner, so that each of the carrier drop 10 in the stream 33 sequentially collides with at least drops that aggregately comprise one pre-determined amount and combination of APIs and/or excipients from the set and are dispensed from some of the at least two liquid dispensers 2,3.

Item 113 may include the device of the item 111 or 112 or some other item herein, wherein the stream 33 comprises a first carrier drop 10, a second carrier drop 10a, and at least a third carrier drop 10b, and the plurality 44 comprising of at least the liquid dispensers 1-3 is configured so that the first carrier drop 10 collides with drops that aggregately comprise a first pre-determined amount and combination of APIs and/or excipients, the second carrier drop 10a collides with drops that aggregately comprise a second pre-determined amount and combination of APIs and/or excipients, the third carrier drop 10b collides with drops that aggregately comprise a third pre-determined amount and combination of APIs and/or excipients, and so on, until all of the carrier drops 10 (10a, 10b . . . ) in the stream 33 collide with at least some drops that aggregately comprise any desired pre-determined amount and combination of APIs and/or excipients from the set, all of the carrier drops 10 (10a, 10b . . . ) in the stream 33 land on the medical device positioned on the support surface 23, and a variable dosing of APIs and/or excipients is achieved across the select XY coordinates on the medical device.

Item 114 may include the device of any one of the items 111-113 or some other item herein, wherein the at least two liquid dispensers 2,3 are arranged in an array, such as a linear array or a circular array; or at least some of the liquid dispensers are arranged in at least a first array 11 and a second array 12, wherein the first array 11 and the second array 12 are configured to dispense drops towards a single stream 33 of the carrier drops 10 (10a, 10b . . . ), or the first array 11 is configured to dispense drops towards a first stream 33 of carrier drops 10 (10a, 10b . . . ), and the second array 12 is configured to dispense drops towards a second stream of carrier drops 10 (10a, 10b . . . ).

Item 115 may include the device of any one of the items 111-114 or some other item herein, wherein the plurality 44 of liquid dispensers is configured to collide the carrier drop 10 with drops that aggregately contain more than two APIS and/or excipients from the set.

Item 116 may include the device of any one of the items 111-115 or some other item herein, wherein the support surface 23 is positioned so that two or more carrier drops 10 (10a, 10b . . . ) from the stream 33 land at a single select XY coordinate whereat one pre-manufactured placebo is located, wherein the pre-determined amount of APIs and/or excipients is contained in the two or more carrier drops 10 (10a, 10b . . . ).

Item 117 may include the device of any one of the items 111-116 or some other item herein, wherein the plurality 44 of liquid dispensers is configured so that each API from the set is suspended in a liquid in a single container that supplies the liquid to at least two liquid dispensers 2,3 in the plurality 44 of liquid dispensers, and each excipient from the set is suspended in another liquid in another container, which supplies the another liquid to another at least two liquid dispensers 2,3 in the plurality 44 of liquid dispensers.

Item 118 may include the device of any one of the items 111-117 or some other item herein, wherein each of the liquid dispensers in the plurality 44 of liquid dispensers comprises at least a container for holding liquids that include one API and/or excipient from the set to dispense, a drop generating element positioned and configured to cause at least one drop to dispense, a nozzle through which the at least one drop is ejected from, a capillary which connects the container with the nozzle so that the liquid runs from the container through the capillary and out through the nozzle for the at least one drop to be dispensed, and synchronisation means, which enable each of the liquid dispensers to operate in a synchronised manner with other liquid dispensers in the plurality 44 of liquid dispensers; and/or a single container is configured to supply liquids to two or more nozzles in the plurality 44 of liquid dispensers; and/or two or more containers are configured to supply liquids to a single nozzle in the plurality 44 of liquid dispensers.

Item 119 may include the device of any one of the items 111-118 or some other item herein, wherein the device comprises a mechanisation 22 configured to move the support surface 23 in at least one direction relative to the plurality 44 of liquid dispensers, and/or move the plurality 44 of liquid dispensers in at least one direction relative to the support surface 23.

Item 120 may include the device of any one of the items 111-119 or some other item herein, wherein the device comprises a mixing chamber 50, which is a partially or a fully closed compartment of the device wherein the plurality 44 of liquid dispensers and at least one support surface 23 are positioned, and the mixing chamber 50 is the fully closed compartment during the time in which the drops are in-flight.

Item 121 may include the device of any one of the items 111-120 or some other item herein, wherein the set comprises APIs and/or excipients, such as: APIs, polymers (such as: hydroxypropyl methyl cellulose (HPMC), poly vinyl alcohol (PVA), Eudragit® derivates, polyvinylpyrroldone (PVP), polyethylene glycol (PEG), Soluplus®), surfactants (such as: polysorbate 20—Tween 20®, polysorbate 80—Tween 80® or other), liquid ink carriers (such as: water, ethanol, propanol, dimethyl sulfoxide (DMSO) or any combination of liquid ink carriers), humectants (such as: PEG, glycerol, or other), viscosity modifiers (such as: glycerol, PEG, or other), or other ingredients to apply to the medical device or the micro-needles.

Item 122 may include the device of any one of the items 111-121 or some other item herein, wherein multiple thin film layers of carrier drops 10 (10a, 10b . . . ) are deposited on the medical device and a variable dosing of APIs and/or excipients is achieved across the multiple thin film layers applied on the medical device.

Item 123 may include the device of any one of the items 111-122 or some other item herein, wherein the medical device positioned on the support surface 23 is any medical device, such as: at least one implant, a micro-needle patch or any other medical device.

Item 124 may include the device of any one of the items 111-123 or some other item herein, wherein the device comprises electric plates 21 arranged perpendicularly to a path of at least one drop dispensed from at least one liquid dispenser, wherein the electric plates 21 have holes through which the at least one drop passes, the at least one liquid dispenser is configured to provide an electric charge to the at least one drop, and the electric plates 21 are configured so that the electric plates 21 exert electric fields on the at least one drop in a synchronised manner, so that the at least one drop is manipulated with the electric fields exerted by the electric plates 21 during the time in which the at least one drop passes through the electric fields exerted by the electric plates 21

Item 125 may include a method for applying active pharmaceutical ingredients (hereinafter: APIs) and/or excipients on medical devices, the method including dispensing and colliding drops by using at least a plurality of liquid dispensers 1-3, so that a stream 33 of carrier drops 10 (10a, 10b . . . ) is dispensed and each of the carrier drops 10 (10a, 10b . . . ) collides and merges with at least drops that aggregately comprise one subset of APIs and/or excipients from a set during flight, and collecting the carrier drops 10 (10a, 10b . . . ), by using a support surface 23, in a series of select XYZ coordinates on a medical device located on the support surface 23, so that at least one subset of APIs and/or excipients is applied on the medical device.

Item 126 may include the method for applying APIs and/or excipients on medical devices by using the device in any of the items 111-124 or some other item herein.

Item 127 may include a device for assembly of nucleic acids, the device comprising a plurality 44 of liquid dispensers 1-3 being configured to dispense drops, wherein at least a carrier drop 10 sequentially collides with at least drops that aggregately comprise a subset of nucleic acids from a set, so that the subset of nucleic acids is located in the carrier drop 10, wherein the subset of nucleic acids comprises all components to produce final nucleic acid molecules, and a support surface 23 configured to collect the carrier drop 10.

Item 128 may include the device of the item 127 or some other item herein, wherein at least one liquid dispenser 1 in the plurality 44 is configured to dispense the carrier drop 10, and/or the carrier drop 10 has a trajectory that passes by at least two liquid dispensers 2,3 configured to dispense drops towards the trajectory of the carrier drop 10 in a synchronised manner, so that the carrier drop 10 sequentially collides with at least all of the drops that contain components from the subset and are dispensed from at least some of the at least two liquid dispensers 2,3.

Item 129 may include the device of the item 127 or 128 or some other item herein, wherein at least one liquid dispenser is configured to dispense a stream 33 of carrier drops 10 (10a, 10b . . . ), wherein the stream 33 comprises a first carrier drop 10, a second carrier drop 10a, and at least a third carrier drop 10b, and the plurality 44 comprising of at least the liquid dispensers 1-3 is configured so that the first carrier drop 10 collides with at least drops that aggregately comprise components from a first subset, the second carrier drop 10a collides with at least drops that aggregately comprise components from a second subset, the third carrier drop 10b collides with at least drops that aggregately comprise components from a third subset, and so on, until all of the carrier drops 10 (10a, 10b . . . ) in the stream 33 collide with at least some drops that aggregately comprise at least some components from the set, and all of the carrier drops 10 (10a, 10b . . . ) in the stream 33 land on the support surface 23.

Item 130 may include the device of any one of the items 127-129 or some other item herein, wherein the at least two liquid dispensers 2,3 are arranged in an array, such as a linear array or a circular array; or at least some of the liquid dispensers are arranged in at least a first array 11 and a second array 12, wherein the first array 11 and the second array 12 are configured to dispense drops towards a single stream 33 of the carrier drops 10 (10a, 10b . . . ), or the first array 11 is configured to dispense drops towards a first stream 33 of carrier drops 10 (10a, 10b . . . ), and the second array 12 is configured to dispense drops towards a second stream of carrier drops 10 (10a, 10b . . . ).

Item 131 may include the device of any one of the items 127-130 or some other item herein, wherein each of the liquid dispensers in the plurality 44 of liquid dispensers is configured to dispense up to a hundred thousand (100000) drops per second, and/or is configured to dispense drops with a volume in a range between one (1) picoliter to a hundred (100) nanoliters; and/or the plurality 44 of liquid dispensers comprises fifty or more, or five hundred or more, or ten thousands or more liquid dispensers.

Item 132 may include the device of any one of the items 127-131 or some other item herein, wherein the support surface 23 is: made of a bendable material or a non-bendable material, and/or is detachable or non-detachable relative to the device, and/or is a container filled with a liquid in which the carrier drops 10 (10a, 10b . . . ) land, and/or is a surface.

Item 133 may include the device of any one of the items 127-132 or some other item herein, wherein the plurality 44 of liquid dispensers are configured to dispense drops of a first liquid, and the support surface 23 is filled or covered with a second liquid, and the first liquid and the second liquid are immiscible.

Item 134 may include the device of any one of the items 127-133 or some other item herein, wherein the device comprises electric plates 21 arranged perpendicularly to a path of at least one drop dispensed from at least one liquid dispenser, wherein the electric plates 21 have holes through which the at least one drop passes, the at least one liquid dispenser is configured to provide an electric charge to the at least one drop, and the electric plates 21 are configured so that the electric plates 21 exert electric fields on the at least one drop in a synchronised manner, so that the at least one drop is manipulated with the electric fields exerted by the electric plates 21 during the time in which the at least one drop passes through the electric fields exerted by the electric plates 21.

Item 135 may include the device of any one of the items 127-134 or some other item herein, wherein the device comprises a mixing chamber 50, which is a partially or a fully closed compartment of the device wherein the plurality 44 of liquid dispensers and at least one support surface 23 are positioned, and the mixing chamber 50 is the fully closed compartment during the time in which the drops are in-flight.

Item 136 may include the device of any one of the items 127-135 or some other item herein, wherein the carrier drop 10 includes binding reagents, which enable an assembly and binding of the components located in the carrier drop 10, wherein the binding reagents are included in the carrier drop 10 or provided by a drop that collides and merges with the carrier drop 10 during flight.

Item 137 may include the device of any one of the items 127-136 or some other item herein, wherein the device comprises a processing chamber 60 having at least one processing element 401 configured to change a temperature in the processing chamber 60 over time so that a sequence of temperatures is achieved over time in the processing chamber 60, and the sequence of temperatures causes a biochemical reaction to occur between the components and binding reagents located in each carrier drop 10 positioned in the processing chamber 60, wherein the biochemical reaction causes the components located in each of the carrier drops 10 (10a, 10b . . . ) to assemble and bind together to form the final nucleic acid molecules, wherein the biochemical reaction is either a Polymerase Chain Reaction, or a Sticky End Ligation, or a Cell Free Cloning, or a BioBricks Assembly, or a Gibson Assembly, or a HiFi Assembly, and wherein at least some of the biochemical reactions include multiplication of the final nucleic acid molecules.

Item 138 may include the device of any of the items 127-137 V, wherein the device comprises a mechanisation 22 configured to move the support surface 23 in at least one direction relative to the plurality 44 of liquid dispensers, and/or move the plurality 44 of liquid dispensers in at least one direction relative to the support surface 23, and/or move the support surface 23 from the mixing chamber 50 to the processing chamber 60.

Item 139 may include the device of any one of the items 127-138 or some other item herein, wherein each of the liquid dispensers in the plurality 44 of liquid dispensers comprises at least a container for holding liquids to dispense, a drop generating element positioned and configured to cause at least one drop to dispense, a nozzle through which the at least one drop is ejected from, a capillary which connects the container with the nozzle so that the liquid runs from the container through the capillary and out through the nozzle for the at least one drop to be dispensed, and synchronisation means, which enable each of the liquid dispensers to operate in a synchronised manner with other liquid dispensers in the plurality 44 of liquid dispensers; and/or a single container is configured to supply liquids to two or more nozzles in the plurality 44 of liquid dispensers; and/or two or more containers are configured to supply liquids to a single nozzle in the plurality 44 of liquid dispensers.

Item 140 may include a method for assembly of nucleic acids, the method including dispensing, by using a plurality 44 of at least liquid dispensers 1-3, at least one carrier drop 10 and at least drops that aggregately comprise a subset of components from a set of nucleic acid components, wherein the carrier drop 10 sequentially collides with at least drops that aggregately comprise the subset of components, so that the subset of components is located in the carrier drop during flight, and collecting the at least one carrier drop 10 by using a support surface 23.

Item 141 may include a method for assembly of nucleic acids using the device of any one of the items 127-139 or some other item herein.

1. (canceled)

2. The device according to claim 9, wherein at least one liquid dispenser is configured to dispense the carrier drop, and/or the carrier drop has a trajectory that passes by at least two liquid dispensers configured to dispense drops towards the trajectory of the carrier drop in a synchronized manner, so that the carrier drop sequentially collides with at least all drops that include liquids from the sub-plurality of liquid dispensers and are dispensed by at least some of the at least two liquid dispensers.

3. The device according to claim 9, wherein at least one liquid dispenser is configured to dispense a stream of carrier drops, wherein the stream comprises a first carrier drop, a second carrier drop, and at least a third carrier drop, and the plurality of liquid dispensers is configured so that the first carrier drop collides with drops from a first sub-plurality of liquid dispensers, the second carrier drop collides with drops from a second sub-plurality of liquid dispensers, the third carrier drop collides with drops from a third sub-plurality of liquid dispensers, and so on, until all of the carrier drops in the stream collide with at least some drops dispensed from at least some liquid dispensers from the plurality of liquid dispensers, and all of the carrier drops in the stream land on the support surface.

4. The device according to claim 9, wherein the at least two liquid dispensers are arranged in an array, such as a linear array or a circular array; or at least some of the liquid dispensers are arranged in at least a first array and a second array, wherein the first array and the second array are configured to dispense drops towards a single stream of the carrier drops, or the first array is configured to dispense drops towards a first stream of carrier drops, and the second array is configured to dispense drops towards a second stream of carrier drops.

5. The device according to claim 9, wherein each of the liquid dispensers in the plurality of liquid dispensers is configured to dispense up to a hundred thousand (100,000) drops per second, and/or is configured to dispense drops with a volume in a range between 1 picoliter to 100 nanoliters; and/or the plurality of liquid dispensers comprises fifty or more, or five hundred or more, or ten thousands or more liquid dispensers.

6. The device according to claim 9, wherein the support surface is: made of a bendable material or a non-bendable material, and/or is detachable or non-detachable relative to the device, and/or is a container filled with a liquid in which the carrier drops land, and/or is a surface.

7. The device according to claim 9, wherein the plurality of liquid dispensers are configured to dispense drops of a first liquid, and the support surface is filled or covered with a second liquid, and the first liquid and the second liquid are immiscible.

8. (canceled)

9. A device for mixing liquids, the device comprising:
a plurality of liquid dispensers configured to dispense drops, wherein at least a carrier drop sequentially collides and merges with drops dispensed from a sub-plurality of liquid dispensers, so that liquids from the sub-plurality of liquid dispensers are located in the carrier drop;
a support surface being positioned so that the carrier drop lands on the support surface; and
a mixing chamber, which is a partially or a fully closed compartment of the device wherein the plurality of liquid dispensers and the support surface are positioned, and the mixing chamber is the fully closed compartment during the time in which the drops are in-flight.

10. The device according to claim 9, wherein the device further comprises electric plates arranged perpendicularly to a path of at least one drop dispensed from at least one liquid dispenser, wherein the electric plates have holes through which the at least one drop passes, the at least one liquid dispenser is configured to provide an electric charge to the at least one drop, and the electric plates are configured so that the electric plates exert electric fields on the at least one drop in a synchronised manner, so that the at least one drop is manipulated with the electric fields exerted by the electric plates during the time in which the at least one drop passes through the electric fields exerted by the electric plates.

11. (canceled)

12. The device according to claim 9, wherein the device further comprises a mechanisation configured to move the support surface in at least one direction relative to the plurality of liquid dispensers, and/or move the plurality of liquid dispensers in at least one direction relative to the support surface, and/or move the support surface from the mixing chamber to the processing chamber.

13. The device according to claim 9, wherein each of the liquid dispensers in the plurality of liquid dispensers comprises at least a container for holding liquids to dispense, a drop generating element positioned and configured to cause at least one drop to dispense, a nozzle through which the at least one drop is ejected from, a capillary which connects the container with the nozzle so that the liquid runs from the container through the capillary and out through the nozzle for the at least one drop to be dispensed, and synchronisation means, which enable each of the liquid dispensers to operate in a synchronised manner with other liquid dispensers in the plurality of liquid dispensers; and/or a single container is configured to supply liquids to two or more nozzles in the plurality of liquid dispensers; and/or two or more containers are configured to supply liquids to a single nozzle in the plurality of liquid dispensers.

14. (canceled)

15. A method for mixing liquids using a device comprising:
a plurality of liquid dispensers configured to dispense drops, wherein at least a carrier drop sequentially collides and merges with drops dispensed from a sub-plurality of liquid dispensers, so that liquids from the sub-plurality of liquid dispensers are located in the carrier drop:
a support surface being positioned so that the carrier drop lands on the support surface; and
a mixing chamber, which is a partially or a fully closed compartment of the device wherein the plurality of liquid dispensers and the support surface are positioned, and the mixing chamber is the fully closed compartment during the time in which the drops are in-flight:
wherein the method comprises:
dispensing a carrier drop from said plurality of liquid dispensers:
merging the carrier drop with drops from the sub-plurality of liquid dispensers; and
landing the carrier drop merged with the drops from the sub-plurality of liquid dispensers on the support surface.

16-30. (canceled)

31. A device for preparing samples for assays, comprising:
a plurality of liquid dispensers being configured to dispense drops, so that at least carrier drop sequentially collides with N drops that comprise a first substance and M drops that comprise at least a second substance, so that the first substance and the second substance are located at a ratio of interest (N:M) and at an amount of interest (N+M) in the carrier drop during flight,
a support surface being configured to collect the carrier drop on a select XY coordinate on the support surface, and in this way, the samples are prepared for an assay on the support surface; and
a processing chamber wherein at least one environment regulator is installed and configured to regulate at least temperature, humidity, and CO₂ concentration in the processing chamber, so that the sample, which is prepared on the support surface and is located in the processing chamber, is incubated.

32-35. (canceled)

36. A device for bioprinting multi-component 3D objects, comprising:
a plurality of liquid dispensers configured to dispense and collide drops, wherein a stream of n carrier drops is dispensed and each of the n carrier drops in the stream sequentially collides with at least drops that aggregately comprise one subset of components from a set;
a support surface being configured so that the n carrier drops in the stream land in a series of n successive XYZ coordinates that represent a multi-component 3D object, so that the multi-component 3D object is bioprinted on the support surface;
wherein at least one liquid dispenser is configured to dispense the stream of the n carrier drops, wherein the stream comprises a first carrier drop, a second carrier drop, and at least a third carrier drop, and the plurality of liquid dispensers are configured so that the first carrier drop collides with at least drops that aggregately comprise a first subset of components, the second carrier drop collides with at least drops that aggregately comprise a second subset of components, the third carrier drop collides with at least drops that aggregately comprise a third subset components from, and so on, until all of the n carrier drops in the stream collide with at least some drops that aggregately comprise at least some appropriate components from the set, and all of the n carrier drops in the stream land on the support surface.

37-65. (canceled)

66. A device for manufacturing multi-substance tablets, comprising:
a plurality of liquid dispensers configured to dispense and collide drops, wherein a stream of n carrier drops is dispensed and each of the n carrier drops in the stream sequentially collides with at least drops that aggregately comprise one subset of active pharmaceutical ingredients (hereinafter: APIs) and/or excipients from a set;
a support surface being configured so that the n carrier drops in the stream land in a series of n successive XYZ coordinates that represent a plurality of multi-substance tablets, so that the plurality multi-substance tablets is 3D printed on the support surface; and
wherein at least one liquid dispenser is configured to dispense the stream of the n carrier drops, wherein the stream comprises a first carrier drop, a second carrier drop, and at least a third carrier drop, and the plurality of liquid dispensers are configured so that the first carrier drop collides with at least drops that aggregately comprise a first subset of APIs and/or excipients, the second carrier drop collides with at least drops that aggregately comprise a second subset of APIs and/or excipients, the third carrier drop collides with at least drops that aggregately comprise a third subset of APIs and/or excipients from, and so on, until all of the n carrier drops in the stream collide with at least some drops that aggregately comprise at least some appropriate APIs and/or excipients from the set, and all of the n carrier drops in the stream land in the series of n successive XYZ coordinates on the support surface.

67-78. (canceled)

79. A method for manufacturing multi-substance tablets using a device comprising:
a plurality of liquid dispensers configured to dispense and collide drops, wherein a stream of n carrier drops is dispensed and each of the n carrier drops in the stream sequentially collides with at least drops that aggregately comprise one subset of active pharmaceutical ingredients (hereinafter: APIs) and/or excipients from a set:
a support surface being configured so that the n carrier drops in the stream land in a series of n successive XYZ coordinates that represent a plurality of multi-substance tablets, so that the plurality multi-substance tablets is 3D printed on the support surface; and
wherein at least one liquid dispenser is configured to dispense the stream of the n carrier drops, wherein the stream comprises a first carrier drop, a second carrier drop, and at least a third carrier drop, and the plurality of liquid dispensers are configured so that the first carrier drop collides with at least drops that aggregately comprise a first subset of APIs and/or excipients, the second carrier drop collides with at least drops that aggregately comprise a second subset of APIs and/or excipients, the third carrier drop collides with at least drops that aggregately comprise a third subset of APIs and/or excipients from, and so on, until all of the n carrier drops in the stream collide with at least some drops that aggregately comprise at least some appropriate APIs and/or excipients from the set, and all of the n carrier drops in the stream land in the series of n successive XYZ coordinates on the support surface;
wherein the method comprises:
dispensing the stream of n carrier drops;
colliding each of the n carrier drops in the stream with the at least drops that aggregately comprise the one subset of APIs and/or excipients from the set;
landing the n carrier drops in the stream in the series of n successive XYZ coordinates so that the plurality multi-substance tablets are 3D printed on the support surface.

80-141. (canceled)
